(19) United States
(12) Patent Application Publication
(10) Pub. No.: US 2025/0160697 A1
(43) Pub. Date: May 22, 2025

(54) SENSING SYSTEMS AND METHODS FOR HYBRID GLUCOSE AND KETONE MONITORING

(71) Applicant: Dexcom, Inc., San Diego, CA (US)

(72) Inventor: Joshua Ray WINDMILLER, San Diego, CA (US)

(21) Appl. No.: 19/030,851

(22) Filed: Jan. 17, 2025

Related U.S. Application Data
(63) Continuation of application No. 18/401,163, filed on Dec. 29, 2023.
(60) Provisional application No. 63/478,005, filed on Dec. 30, 2022.

(51) Int. Cl. A61B 5/145; A61B 5/00; A61B 5/0205; A61B 5/1486; G16H 40/67; G16H 50/30

(52) U.S. Cl. CPC A61B 5/14532; A61B 5/0004; A61B 5/0205; A61B 5/14546; A61B 5/1486; A61B 5/4848; A61B 5/7275; A61B 5/746; G16H 40/67; G16H 50/30

(57) ABSTRACT

Certain aspects of the present disclosure relate to a monitoring system comprising a continuous analyte sensor configured to generate analyte measurements associated with analyte levels of a patient, and a sensor electronics module coupled to the continuous analyte sensor and configured to receive and process the analyte measurements.

### CROSS-REFERENCE TO RELATED APPLICATIONS

This application is a continuation of U.S. application Ser. No. 18/401,163, filed Dec. 29, 2023, which claims benefit of and priority to U.S. Provisional Application No. 63/478,005, filed Dec. 30, 2022, which is assigned to the assignee hereof and hereby expressly incorporated herein in its entirety as if fully set forth below and for all applicable purposes.

### INTRODUCTION

Diabetes is a metabolic condition relating to the production or use of insulin by the body. Insulin is a hormone that allows the body to use glucose for energy, or store glucose as fat.

When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low (dysglycemia). Regulation of blood glucose levels depends on the production and use of insulin, which facilitates the movement of blood glucose into cells.

When the body does not produce enough insulin, or when the body is unable to effectively use insulin that is present, blood sugar levels can elevate beyond the normal range (e.g., euglycemia). The state of having a higher than normal blood sugar level is called “hyperglycemia.” Chronic hyperglycemia can lead to a number of health problems, such as cardiovascular disease, cataract and other eye problems, nerve damage (neuropathy), liver disease, kidney damage, and amputation. Hyperglycemia can also lead to acute problems, such as diabetic ketoacidosis—a state in which the blood becomes excessively acidic due to elevation in ketones bodies (e.g., acetone, acetoacetate, β-hydroxybutyrate), which are produced as a consequence of the metabolic process of gluconeogenesis. The state of having lower than normal blood glucose levels is called “hypoglycemia.” Severe hypoglycemia can lead to acute crises that can result in seizures or death.

Diabetes conditions are sometimes referred to as “Type 1” and “Type 2.” A Type 1 diabetes patient is typically able to use insulin when it is present, but the body is unable to produce sufficient amounts of insulin on its own, because of reduced function of the insulin-producing beta cells of the pancreas. A Type 2 diabetes patient may produce sufficient quantities of insulin, but the patient has become “insulin resistant” due to a reduced sensitivity to insulin at the cellular level. The result is that even though insulin is present in the body, the insulin is not sufficiently or is ineffectively used by the patient's cells to effectively uptake glucose, thereby resulting in chronically elevated levels of glucose in the blood and/or interstitial fluid. A diabetes patient can receive insulin to manage blood glucose levels. Insulin can be received, for example, through a manual injection with a needle. Wearable insulin pumps are also available. Diet and exercise also affect blood glucose levels.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example health management system that may be used in connection with implementing embodiments of the present disclosure.

FIG. 2 is a diagram conceptually illustrating an example of a continuous analyte monitoring system including an example of a continuous analyte sensor(s) with sensor electronics, in accordance with certain aspects of the present disclosure.

FIG. 3 illustrates example inputs and example metrics that are calculated based on the inputs for use by the health management system of FIG. 1, according to some embodiments disclosed herein.

FIG. 4 is an example workflow for generating one or more decision support recommendations, according to certain embodiments of the present disclosure.

FIG. 5 is a flow diagram depicting a method for training machine learning models to provide decision support recommendations, according to certain embodiments of the present disclosure.

FIG. 6 is a block diagram depicting a computing device configured to perform the operations of FIGS. 4 and/or 5, according to certain embodiments disclosed herein.

FIGS. 7A-7B illustrate exemplary enzyme domain configurations for a continuous multi-analyte sensor, according to certain embodiments disclosed herein.

FIGS. 7C-7D illustrate exemplary enzyme domain configurations for a continuous multi-analyte sensor, according to certain embodiments disclosed herein.

FIG. 7E illustrates an exemplary enzyme domain configurations for a continuous multi-analyte sensor, according to certain embodiments disclosed herein.

FIGS. 8A-8B illustrate alternative views of an exemplary dual electrode enzyme domain configuration for a continuous multi-analyte sensor, according to certain embodiments disclosed herein.

FIGS. 8C-8D illustrate alternative views of an exemplary dual electrode enzyme domain configuration for a continuous multi-analyte sensor, according to certain embodiments disclosed herein.

FIG. 8E illustrates an exemplary dual electrode configuration for a continuous multi-analyte sensor, according to certain embodiments disclosed herein.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

To keep blood glucose levels in range, patients are typically instructed to better manage their diet and exercise and/or administer different types of medication, such as insulin, sulfonylureas, and metformin, which can be administered, for example, via the oral route, via manual injections, or through body-worn automated insulin delivery devices.

Another medication used for diabetes includes sodium glucose cotransporter 2 (SGLT2) inhibitors which are a class of medicines that are approved to lower blood sugar for those with type 2 diabetes. SGLT2 inhibitors are available as single-ingredient products and also in combination with other medicines (i.e., co-formulated with insulin). SGLT2 inhibitors lower blood sugar by blocking glucose absorption and causing the kidneys to remove sugar from the body through the urine, thereby, helping patients achieve euglycemia. However, SGLT2 inhibitors can also elevate the body's ketone levels beyond safe levels. As such, diabetic patients using SGLT2 inhibitors experience an increased risk of euglycemic diabetic ketoacidosis (euDKA). In particular, the euglycemia state induced by SGLT2 inhibitors can conceal the underlying euDKA, thereby, creating difficulty in identifying euDKA, which can lead to dangerous and life-threatening conditions. As a result, although high glucose levels persisting over a long period of time could typically be indicative of a higher risk of euDKA, for patients using SGLT2 inhibitors, glucose levels can no longer be relied on for understanding the risk profile for developing euDKA.

Existing body-worn diabetes management systems (e.g., continuous glucose monitors, CGMs) that assist diabetic patients with managing their diabetes suffer from multiple deficiencies, thereby leaving these systems with an inability to help diabetic patients on SGLT2 inhibitors manage an increased risk of euDKA. First, existing diabetes management systems do not provide a continuous ketone sensor in conjunction with a continuous glucose sensor to allow for ketone levels to be continuously monitored even when patients are in a euglycemia state induced in response to usage of SGLT2 inhibitors. Second, existing diabetes management systems do not provide any software application able to receive and analyze both glucose and ketone measurements to identify an elevated risk of euDKA. As such, existing diabetes management software applications give patients a potentially harmful false sense of security, causing the patients to believe they are in a healthy state because their glucose levels are in range. Third, without the ability to receive and analyze ketone and glucose measurements from ketone and glucose sensors, existing software applications are not in possession of a complete picture of the patient's physiologic or metabolic state status and, thereby, not able to provide effective treatment options to reduce the risk of euDKA. For example, existing diabetes systems are not able to analyze and determine how well a certain dosage of a SGLT2 inhibitor that's prescribed to a patient is tolerated by that patient and how the dosage can be titrated or optimized to not only help the patient achieve euglycemia but also minimize the risk of euDKA.

Accordingly, the embodiments herein provide a technical solution to the technical problems described above by providing a health management system, including a continuous analyte monitoring system that may include one or more continuous analyte sensors and/or one or more non-analyte sensors. As used herein, the term “continuous” may mean fully continuous, semi-continuous, periodic, etc. analyte monitoring, which may provide a fully continuous, semi-continuous, or periodic stream of analyte data, for example.

The one or more analyte sensors may be used for generating glucose values and ketone values indicative of glucose and ketone levels in a patient. The one or more analyte sensors may include a single sensor configured to generate both the glucose values and the ketone values or may include a sensor array of two or more distinct sensors in which one sensor generates glucose values and another sensor generates ketone values while still other analyte sensors may be included to generate analyte values in addition to glucose and ketone values in both the single sensor and two or more sensor embodiments. The analyte sensor system may be configured to communicate the glucose and ketone values to a user display device. Examples of non-analyte sensors may include sensors for measuring blood pressure, oxygen saturation, heart rate, respiration rate, body temperature, kinesthetic parameters (e.g., pace, steps, gait), renal function, hydration, or the like.

The health management system also includes a user display device. The user display device may include a processor configured to execute a software application for receiving and analyzing the glucose and ketone values, along with other data (e.g., non-analyte sensor data). The software application may then utilize a fusion model described below to take at least a fusion of the user's glucose and ketone values as input and provide to the user a decision support output. The decision support output may take a variety of forms and types.

As an example, a user that has been prescribed a certain dosage and frequency of SGLT2 inhibitors may be directed by their physician to use the health management system described above in order to determine whether the prescribed dosage/frequency of SGLT2 inhibitors is safe and well-tolerated by predicting a likelihood of euDKA. In such an example, the user utilizes the analyte sensor system, which continuously generates at least glucose and ketone values, as well as a software application that receives the glucose and ketone measurements, to be used an input into a fusion model for predicting the likelihood of euDKA occurring within a defined time period (e.g., 1 hour, 1 day, 1 week, 1 month, etc.). In particular, in such examples, the prescribed dosage and frequency of SGLT2 inhibitors as well as the user's own glucose and ketone measurements, and/or other user information are used as input to determine a likelihood of euDKA. In some embodiments, the likelihood of euDKA may include a risk-adjusted score for euDKA, for example, taking into consideration a patient's risk profile (e.g., endogenous factors, such as age, BMI, etc., and/or exogenous factors including other medications such as insulin, readings from non-analyte sensors, etc.). As an example, after a week of taking SGLT2 inhibitors, the fusion model may indicate a 90% likelihood of euDKA occurring within, for example, a week of when the prediction is made. In such an example, the likelihood is displayed to the user and/or transmitted to the physician/prescriber, so that the dosage and/or frequency of the inhibitors can be adjusted to lower the likelihood of euDKA. Examples of other user information include other analyte data (analyte data besides glucose and ketone measurements), non-analyte data (e.g., blood pressure, temperature, oxygen saturation, heart rate, accelerometer, etc.), demographic information (e.g., age, gender, ethnicity, etc.), anthropometric information (e.g., height, weight, BMI), clinical chemistry information (e.g., fasting blood glucose level, HbA1c level), compliance with SGLT2 therapy, disease information, diet/meal information, exercise/activity data, renal state information, hydration, body mass index (BMI), etc.

In another example, a user that has been prescribed a certain dosage and frequency of SGLT2 inhibitors may be directed by their physician to use the health management system described above in order to predict an optimal dosage/frequency of the inhibitors. In such an example, the user utilizes the analyte sensor system, which continuously generates at least glucose and ketone values, as well as a software application that receives the glucose and ketone measurements to be used an input into a fusion model for predicting the optimal dosage/frequency of the inhibitors. In particular, in that example, the user's own glucose and ketone measurements, and/or other user information, are used as input to predict a dosage/frequency for the user that minimizes the risk of euDKA while helping the user achieve euglycemia or otherwise maximize time-in-range.

In another example, a model-based unsupervised learning system is invoked to ingest glucose and/or ketone analyte data in combination with, optionally, non-analyte data, in order to build a customized metabolic model for the user. The metabolic model can be constructed without any stimulus (passive ingestion of data) or can be constructed using a controlled stimulus such as an oral glucose tolerance test, an oral ketone tolerance test (ketone esters), a specific dosage of an SGLT2 inhibitor, or a specific dosage of insulin. The metabolic model would then be used by the health management system described above in order to predict an optimal dosage/frequency of the inhibitors. In particular, in this example, the user's own glucose and ketone measurements, and/or other user information, are used as input to predict a dosage/frequency for the user that minimizes the risk of euDKA while helping the user achieve euglycemia or otherwise maximize time-in-range.

While embodiments herein may refer to specific therapies such as insulin, inhibitors such as SGLT2, agonists such as GLP-1, etc., one of skill in the art will appreciate that embodiments described herein may provide recommendations for patients under therapy with other pharmacologic agents. Embodiments may provide more accurate titration of pharmacologic agents for improved treatment for patients with narrow or evolving treatment situations which may be based on previous treatment responsiveness and/or disease/condition type and/or progression.

In certain embodiments, the fusion model may include one of a variety of algorithms. For example, the fusion model may be a rule-based model, an artificial intelligence (AI) model, such as a machine-learning model (e.g., classification or regression models), a Kalman filter, a probabilistic model (e.g., Bayesian Network), a stochastic model (e.g., Gaussian models), and the like. The rule-based models and/or machine-learning models may take into account one or more inputs and/or metrics for the user when providing the decision support outputs described above.

For example, a rule-based model may predict the risk or likelihood of a patient experiencing euDKA or an optimal dosage/frequency of the inhibitors. Rule-based models involve using a set of rules for mapping certain inputs to certain outputs. These rules are sometimes referred to as ‘If statements’ as they tend to follow the line of ‘If X happens, then do or conclude Y.’ In particular, the fusion model may apply rule-statements (e.g., if-then statements) to determine the risk or likelihood of a patient developing euDKA while under SGLT2 therapy or an optimal dosage/frequency of the inhibitors. These rule-statements may comprise, among other things, absolute levels of glucose and/or ketones, rate of change of glucose and/or ketones (e.g., physiologic vs pathologic rates of change), inflection points in the glucose and/or ketone time series data, ratios of absolute glucose values to absolute lactate values, and cross-correlation of glucose and ketone time series data.

Such rules may be defined and maintained in a reference library or lookup table. For example, the reference library may maintain ranges of analyte (e.g., glucose, ketone, etc.) levels and ranges of analyte level rates of change (and/or other analyte data) and/or other analyte metrics, which may be mapped to, e.g., different euDKA risk stratifications or different dosage/frequency of the inhibitors. In certain embodiments, such rules may be determined based on empirical research or an analysis of historical records, such as the records of historical patients stored in a historical records database. In some cases, the reference library may become very granular. For example, other factors may be used in the reference library to create such “rules”. Other factors may include gender, age, diet, activity, blood pressure, renal state, hydration, oxygen saturation, disease history, family disease history, body mass index (BMI), etc. Increased granularity may provide more accurate outputs. In certain embodiments, the reference library may comprise an embedded memory within a microprocessor located within the body-worn CGM/CKM.

In embodiments where a rule-based model is used to predict a likelihood of euDKA in a patient, the prescribed dosage/frequency prescribed to the user, the user's analyte and non-analyte data, and other types of information described above may be used as input to determine a likelihood of the user experiencing euDKA in a defined time period. In some embodiments, a risk-likelihood score for a euDKA event can be presented by the health management system to the prescriber corresponding to a certain dosage and frequency of SGLT2 given the patient's analyte and non-analyte data. Additionally or alternatively, a prescriber might provide a health management system with a tolerable level of risk of euDKA, and the health management system may, in turn and based on the analyte and non-analyte data fusion model, provide a dosage and/or frequency of SGLT2 corresponding to this level of allowable risk. In embodiments where a rule based model is used to predict an optimal dosage/frequency of the inhibitors, the user's analyte and non-analyte data (e.g., a day, a week, a month of user specific data) and other types of information described above may be used as input to determine an optimal dosage/frequency of the inhibitors.

As an alternative to a rule-based model, a fusion model may be an AI/ML model trained to collect information associated with the user for automatically assessing the risk of euDKA for a user or making a recommendation for changes in a current or proposed SGLT2 therapy for the user. In certain embodiments, a training server system may be configured to train the AI/ML model using training data, which may include population data associated with historical users (e.g., users or non-users of a continuous analyte monitoring system and/or application) previously under SGLT2 therapy.

As another alternative to a rule-based model, a fusion model may be trained to receive glucose and/or ketone measurements and automatically assess the risk of euDKA. For example, known glucose measurements may allow for determination of estimated ketone measurements or known ketone measurements may allow for the determination of estimated glucose measurements. If the user's known or estimated ketone and/or glucose levels remain elevated (e.g., glucose levels above 180 mg/dL or ketone levels above 3 mM), a user may be determined to have, or be at risk of developing, euDKA. Further, an increase in amount of time glucose and/or ketone measurements remain above a certain threshold (e.g., glucose levels above 180 mg/dL or ketone levels above 3 mM) may indicate a user is experiencing euDKA, or developing euDKA and provide treatment options and/or at-home interventions to prevent development or worsening of euDKA.

Further, maximum likelihood estimation may be used to determine a joint probability distribution of glucose and/or ketone measurements. The glucose and ketone signal variances may then be used to calculate weights and, therefore, a weighted average of the glucose and ketone measurements. The weights may be determined as an inverse of the glucose and ketone signal variances. For example, in certain embodiments, the weighted average of the glucose and ketone measurements may be determined based on the following equation: F=EGV*Vg⁻¹+EKV*Vk⁻¹/(Vg⁻¹+Vk⁻¹), where EGV and EKV are the estimated glucose values and estimated ketone values obtained from sensor measurement, respectively, and Vg and Vk represent the glucose and ketone variances, respectively.

In some embodiments, the training data refers to a dataset that has been featurized and labeled. For example, the dataset may include a plurality of data records, each including information corresponding to a different user profile stored in a user database, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model.

As an illustrative example, each relevant characteristic of a user, which is reflected in a corresponding data record, may be a feature used in training the machine learning model. Such features may include demographic related features (e.g., age, gender, ethnicity), anthropometric related features (e.g., height, weight, BMI), clinical chemistry related features (e.g., fasting blood glucose level, metabolic panel results, HbA1c level), analyte related features (e.g., time-stamped analyte values, time-stamped analyte rates of change, change in analyte values (e.g., glucose values, ketone values) of a user under SGLT2 therapy from a first timestamp to a second timestamp, change in analyte baselines of a user under SGLT2 therapy from a first timestamp to a subsequent timestamp, the derivative of the measured linear system of analyte values at a specific timestamp and/or the difference in derivatives to determine rates of change in the slope of the increase or decrease in analyte values of a user under SGLT2 therapy, etc). The data record is also labeled with what the AI/ML is intended to predict. For example, if the AI/ML model is intended to predict the optimal dosage of the inhibitors, then each data record is labeled with the dosage of the inhibitor the corresponding historical user was using. In another example, if the AI/ML model is intended to predict a likelihood of euDKA occurring within a defined time period (e.g., within a week or month), then each data record is labeled with the likelihood of euDKA occurring within the same time period.

The model(s) are then trained using the featurized and labeled training data. In particular, the features of each data record may be used as input into the machine learning model(s), and the generated output may be compared to label(s) associated with the corresponding data record. The model(s) may compute a loss based on the difference between the generated output and the provided label(s). This loss is then used to modify the internal parameters or weights of the model. By iteratively processing each data record corresponding to each historical patient, in certain embodiments, the model(s) may be iteratively refined to reduce the loss and generate accurate predictions associated with a likelihood of euDKA occurring, an optimal dosage/frequency of the inhibitors, etc.

The training server system deploys these trained model(s) to a decision support engine for use during runtime. For example, the decision support may obtain the user profile associated with the user, use information from the user profile as input into the trained model(s), and generate a decision support output. The decision support output may be indicative of a likelihood of euDKA occurring, an optimal dosage/frequency of the inhibitors for the user in real-time or within a certain time, etc. The decision support output may be provided to the user (e.g., through the user display device—smartphone, smartwatch, CGM display), to a user's caretaker (e.g., a parent, a relative, a guardian, a teacher, a nurse, etc.), to a user's physician, or any other individual that has an interest in the wellbeing of the user for purposes of improving the user's health, such as, in some cases, by effectuating the recommended treatment.

As presented above, embodiments of a fusion model may intake data corresponding to the user to generate a decision support output relating to an optimal SGLT2 dosage/frequency and/or a likelihood of an onset of euDKA for the user. Identifying a risk of euDKA occurring or being present in a patient and/or an optimal dosage/frequency of SGLT2 inhibitors to a patient to reduce the risk of euDKA will assist in avoiding a potentially dangerous life-threating physiologic state and costly health event and will alleviate case-load and expense burdens borne by the healthcare provider.

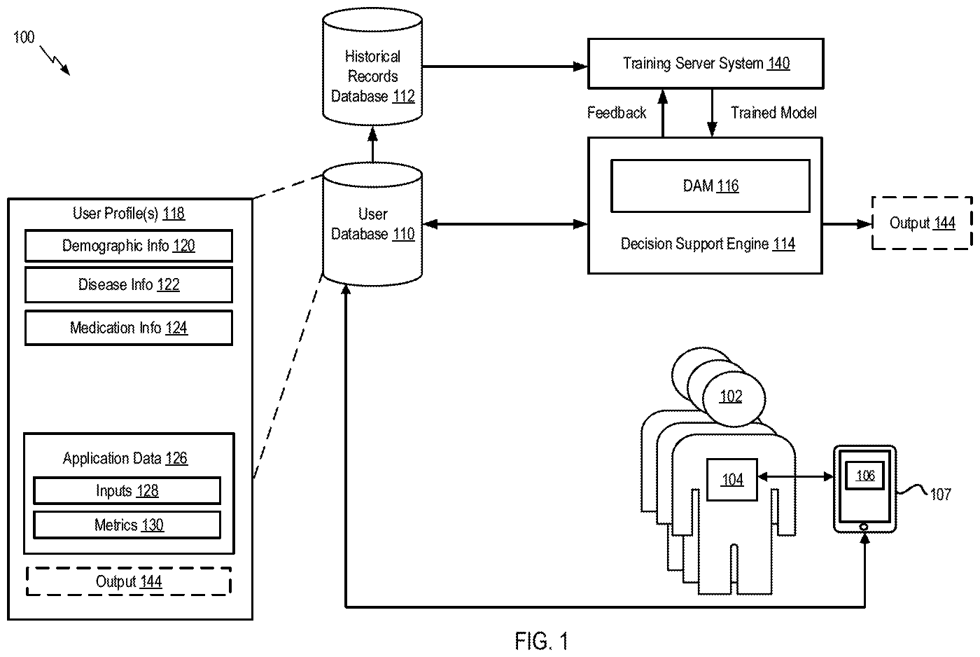

FIG. 1 illustrates an example health management system 100 for providing treatment recommendations, in relation to users 102 (individually referred to herein as a user and collectively referred to herein as users), using a continuous analyte monitoring system 104, including one or more analyte sensors. A user 102, in certain embodiments, may be the patient or, in some cases, the patient's caregiver. In certain embodiments, health management system 100 includes continuous analyte monitoring system 104, a display device 107 that executes application 106, a decision support engine 114, a user database 110, a historical records database 112, a training server system 140, and a decision support engine 114, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid, saliva, mucus, or urine) that can be analyzed. Analytes can include naturally occurring substances, artificial substances, pharmacologic agents, metabolites, ions, blood gasses, hormones, neurotransmitters, vitamins, minerals, peptides, proteins, enzymes, pathogens, toxins, substances of abuse, and/or reaction products. Analytes for measurement by the devices and methods may include, but may not be limited to, ketone, glucose, potassium, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-βhydroxy-cholic acid; cortisol; creatine kinase; creatine kinase MM isoenzyme; cyclosporin A; cystatin C; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sisomicin; somatomedin C; specific antibodies recognizing any one or more of the following that may include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, Leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain implementations. Ions are a charged atoms or compounds that may include the following (sodium, potassium, calcium, chloride, nitrogen, or bicarbonate, for example). The analyte can be naturally present in the biological fluid, for example, a metabolic product, a hormone, an antigen, an antibody, an ion and the like. Alternatively, the analyte can be introduced into the body or exogenous, for example, a contrast agent for imaging, a radioisotope, a chemical agent, a fluorocarbon-based synthetic blood, a challenge agent analyte (e.g., introduced for the purpose of measuring the increase and or decrease in rate of change in concentration of the challenge agent analyte or other analytes in response to the introduced challenge agent analyte), or a drug or pharmaceutical composition, including but not limited to exogenous insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include ketone and glucose, other analytes listed, but not limited to, above may also be considered and measured by, for example, continuous analyte monitoring system 104.

In certain embodiments, continuous analyte monitoring system 104 is configured to continuously measure one or more analytes and transmit the analyte measurements to an electric medical records (EMR) system (not shown in FIG. 1). An EMR system is a software platform which allows for the electronic entry, storage, and maintenance of digital medical data. An EMR system is generally used throughout hospitals and/or other caregiver facilities to document clinical information on patients over long periods. EMR systems organize and present data in ways that assist clinicians with, for example, interpreting health conditions and providing ongoing care, scheduling, billing, and follow up. Data contained in an EMR system may also be used to create reports for clinical care and/or disease management for a patient. In certain embodiments, the EMR may be in communication with decision support engine 114 (e.g., via a network) for performing the techniques described herein. In other embodiments, an EMR may be mined for population-level health statistics, health economics, and the generation of clinical evidence or assessment of healthcare outcomes. In particular, as described herein, decision support engine 114 may obtain data associated with a user, use the obtained data as input into one or more trained model(s), and output a prediction. In some cases, the EMR may provide the data to decision support engine 114 to be used as input into one or more models, e.g., ML models. Further, in some cases, decision support engine 114, after making a prediction, may provide the output prediction to the EMR.

In certain embodiments, continuous analyte monitoring system 104 is configured to continuously measure one or more analytes and transmit the analyte measurements to display device 107 for use by application 106. In some embodiments, the continuous analyte monitoring system 104 may primarily function as a monitoring device by pairing with the display device 107 and transmitting analyte measurements to the display device 107 in a continuous or semi-continuous manner. In other embodiments, the continuous analyte monitoring system 104 may primarily function as a diagnostic device that is configured to store and log the analyte measurements. In such embodiments, the data log stored by the continuous analyte monitoring system 104 may be transmitted to a remote service (e.g., a cloud server) without the involvement of the display device 107. In such embodiments, the continuous analyte monitoring system 104 may be equipped with a mobile internet of things (IoT) interface (e.g., LTE, Cat-M1, NB-IoT, etc.), a cellular radio (e.g., 3G, 4G, LTE, 5G, 6G, etc.), or other means to directly communicate the analyte measurements in the data log to the remote server.

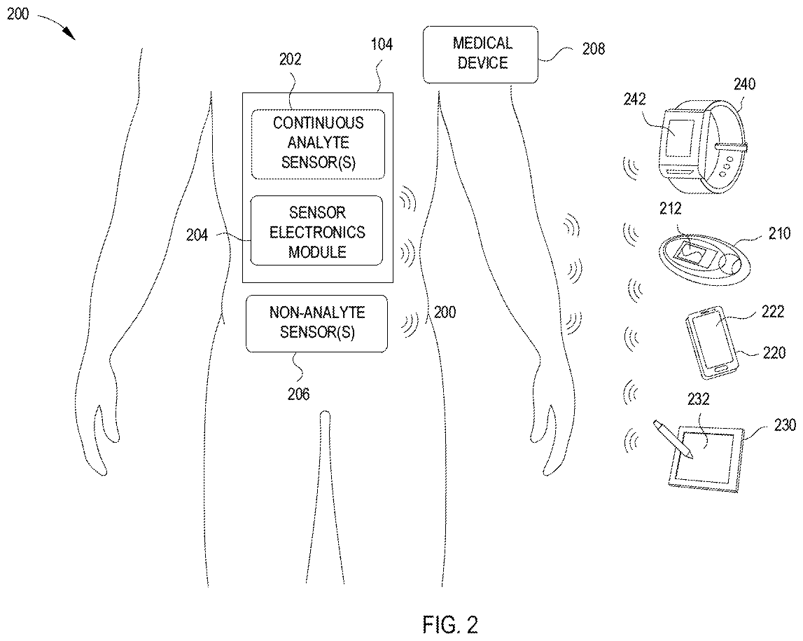

In some embodiments, continuous analyte monitoring system 104 transmits the analyte measurements to display device 107 through a wireless connection (e.g., Bluetooth connection). In certain embodiments, display device 107 is a smart phone. However, in certain other embodiments, display device 107 may instead be any other type of computing device, such as a laptop computer, a smart watch, a tablet, or any other computing device capable of executing application 106. In some embodiments, continuous analyte monitoring system 104 and/or analyte sensor application 106 transmit the analyte measurements to one or more other individuals having an interest in the health of the patient (e.g., a family member or physician for real-time treatment and care of the patient). Continuous analyte monitoring system 104 may be described in more detail with respect to FIG. 2.

Application 106 is a mobile health application that is configured to receive and analyze analyte measurements from continuous analyte monitoring system 104. In particular, application 106 stores information about a user, including the user's analyte measurements, in a user profile 118 associated with the user for processing and analysis, as well as for use by decision support engine 114 to provide decision support recommendations or guidance to the user.

Decision support engine 114 refers to a set of software instructions with one or more software modules, including data analysis module (DAM) 116. In certain embodiments, decision support engine 114 executes entirely on one or more computing devices in a private or a public cloud. In such embodiments, application 106 communicates with decision support engine 114 over a network (e.g., Internet). In some other embodiments, decision support engine 114 executes partially on one or more local devices, such as display device 107, and partially on one or more computing devices in a private or a public cloud. In some other embodiments, decision support engine 114 executes entirely on one or more local devices, such as display device 107. As discussed in more detail herein, decision support engine 114 may provide decision support recommendations to the user via application 106. Decision support engine 114 provides decision support recommendations based on information included in user profile 118.

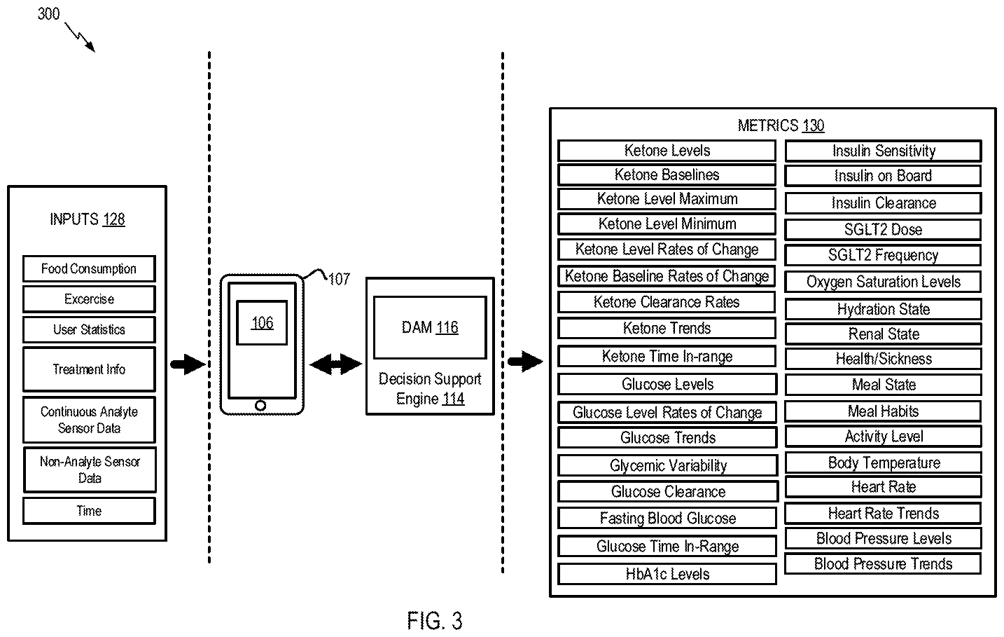

User profile 118 may include information collected about the user from application 106. For example, application 106 provides a set of inputs 128, including the analyte measurements received from continuous analyte monitoring system 104, that are stored in user profile 118. In certain embodiments, inputs 128 provided by application 106 include other data in addition to analyte measurements received from continuous analyte monitoring system 104. For example, application 106 may obtain additional inputs 128 through manual user input, one or more other non-analyte sensors or devices, other applications executing on display device 107, etc. Non-analyte sensors and devices include one or more of, but are not limited to, an insulin pump, an electrocardiogram (ECG) sensor or heart rate monitor, a blood pressure sensor, a respiratory sensor, a thermometer, an oxygenated hemoglobin sensor (spO₂), an activity tracker, a peritoneal dialysis machine, a hemodialysis machine, sensors or devices provided by display device 107 (e.g., accelerometer, inclinometer, gyroscope, camera, global positioning system (GPS), heart rate monitor, etc.) or other user accessories (e.g., a smart watch), or any other sensors or devices that provide relevant information about the user. Inputs 128 of user profile 118 provided by application 106 are described in further detail below with respect to FIG. 3.

DAM 116 of decision support engine 114 is configured to process the set of inputs 128 to determine one or more metrics 130. Metrics 130, discussed in more detail below with respect to FIG. 3, may, at least in some cases, be generally indicative of the health or state of a user, such as one or more of the user's physiological state, trends associated with the health or state of a user, etc. In certain embodiments, metrics 130 may then be used by decision support engine 114 as input for providing guidance to a user. As shown, metrics 130 are also stored in user profile 118.

User profile 118 also includes demographic information 120, disease info 122, and/or medication information (e.g., type of medication, brand of medication, dosage, frequency of administration) 124. In certain embodiments, such information may be provided through user input or obtained from certain data stores (e.g., electronic medical records (EMRs), etc.). In certain embodiments, demographic info 120 may include one or more of the user's age, body mass index (BMI), ethnicity, gender, etc. In certain embodiments, disease info 122 may include information about a condition of a user, such as whether the user has been previously diagnosed with or experienced diabetes, a stage (if known) on a progression from pre-diabetic to a complete lack of insulin production, ketoacidosis, euDKA, diagnosis of other co-morbidities, etc., or had a history of ketoacidosis, euDKA, hyperglycemia, hypoglycemia, co-morbidities, etc. In certain embodiments, information about a user's condition may also include the length of time since diagnosis, the level of control, level of compliance with condition management therapy, other types of diagnosis (e.g., heart disease, obesity) or measures of health (e.g., heart rate, exercise, stress, sleep, etc.), and/or the like.

In certain embodiments, medication information 124 may include information about the amount, frequency, and type of a medication taken by a user. In certain embodiments, the amount, frequency, and type of a medication taken by a user is time-stamped and correlated with the user's analyte levels, thereby indicating the impact the amount, frequency, and type of the medication had on the user's analyte levels. In certain embodiments, medication information 124 may include information about the prescribed dosage/frequency and the consumption of one or more inhibitors (e.g., sodium glucose cotransporter 2 (SGLT2)). Further, medication information 124 may include SGLT2 inhibitor action curves, and/or pharmacokinetic and/or pharmacodynamic properties to determine medication effectiveness, etc. Inhibitors may be prescribed to a patient for the purpose of treating diabetes. The user 102 may be prescribed inhibitors to help control blood glucose levels by blocking absorption of glucose by the body.

As described in more detail below, health management system 100 may be configured to use medication info 124 to determine an inhibitor effectiveness or an optimal inhibitor dosage and frequency to be prescribed to different users. In particular, health management system 100 may be configured to identify one or more optimal prescriptions based on the health of the patient when one or more medications are prescribed, as well as the condition(s) of the patient to be treated.

In certain embodiments, the medication information 124 may include information about consumption of other drugs for the control of blood glucose. For example, the medication information 124 may include metformin, thiazolidinediones, sulfonylureas, GLP-1 receptor agonists, glucagon, and/or insulin action curves, pharmacokinetic and/or pharmacodynamic properties, dosage, and frequency. The medication information 124 may include information manually provided by the user and/or information provided by an automated insulin delivery (AID) device.

In certain embodiments, user profile 118 is dynamic because at least part of the information that is stored in user profile 118 may be revised over time and/or new information may be added to user profile 118 by decision support engine 114 and/or application 106. Accordingly, information in user profile 118 stored in user database 110 provides an up-to-date repository of information related to a user.

User database 110, in some embodiments, refers to a storage server that operates in a public or private cloud. User database 110 may be implemented as any type of datastore, such as relational databases, non-relational databases, key-value datastores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

The user database 110 may include user profiles 118 associated with a plurality of users who similarly interact with application 106 executing on the display devices 107 of the other users. User profiles stored in user database 110 may be accessible to not only application 10 but decision support engine 114 as well. User profiles in the user database 110 may be accessible to the application 106 and the decision support engine 114 over one or more networks (not shown). As described above, the decision support engine 114, and more specifically the DAM 116 of the decision support engine 114, can fetch inputs 128 from the user database 110 and compute a plurality of metrics 130 which can then be stored as application data 126 in the user profile 118, and/or used in population data and statistics as may be required or desired to be used by the application.

In certain embodiments, the user profiles 118 stored in user database 110 may also be stored in a historical records database 112. The user profiles 118 stored in the historical records database 112 may provide a repository of up-to-date information and historical information for each user of the application 106. Thus, the historical records database 112 essentially provides all data related to each user of the application 106, where data is stored according to an associated timestamp. The timestamp associated with information stored in the historical records database 112 may identify, for example, when information related to a user has been obtained and/or updated.

Further, the historical records database 112 may maintain time series data collected for users over a period of time, including for users who use the continuous analyte monitoring system 104 and the application 106. For example, analyte data for a user who has used the continuous analyte monitoring system 104 and the application 106 for a period of five years may have time series analyte data, associated with the user, maintained over the five-year period.

Further, in certain embodiments, the historical records database 112 may also include data for one or more patients who are not users of the continuous analyte monitoring system 104 and/or the application 106. For example, the historical records database 112 may include information (e.g., user profile(s)) related to one or more patients analyzed by, for example, a healthcare physician, or the like, and not previously treated for blood glucose control, as well as information (e.g., user profile(s)) related to one or more patients who were analyzed by, for example, a healthcare physician, or the like, and were previously treated for blood glucose control. Data stored in the historical records database 112 may be referred to herein as population data.

Data related to each patient stored in the historical records database 112 may provide time series data collected over the disease lifetime of the patient. For example, the data may include information about the patient prior to being diagnosed and information associated with the patient during the lifetime of the treatment, including information related to level of treatment required, as well as information related to other diseases or conditions, such as euDKA, adverse events (e.g., hypoglycemia, hypoglycemia, dysglycemia), diabetes, heart conditions and diseases, or similar diseases or other relevant co-morbidities. Such information may indicate symptoms of the patient, physiological states of the patient, ketone levels of the patient, glucose levels of the patient, states/conditions of one or more organs of the patient, habits of the patient (e.g., activity levels, food consumption, etc.), medication prescribed, etc., throughout the lifetime of the treatment.

Although depicted as separate databases for conceptual clarity, in some embodiments, the user database 110 and the historical records database 112 may operate as a single database. In other words, the historical and current data related to users of the continuous analyte monitoring system 104 and the application 106, as well as historical data related to patients that were not previously users of the continuous analyte monitoring system 104 and the application 106, may be stored in a single database. The single database may be a storage server that operates in a public or private cloud or in another arrangement.

As mentioned previously, the health management system 100 is configured to provide a treatment recommendation for a user using the continuous analyte monitoring system 104 including one or more analyte sensors. In certain embodiments, the continuous analyte monitoring system 104 includes, at least a continuous glucose monitor (CGM) and a continuous ketone monitor (CKM). In certain embodiments, the decision support engine 114 is configured to provide real-time and/or non-real-time decision support based on glucose and/or ketone levels to the user and or others, including but not limited to, healthcare providers, family members of the user, caregivers of the user, researchers, artificial intelligence (AI) engines, and/or other individuals, systems, and/or groups supporting care or learning from the data. In particular, the decision support engine 114 may be used to collect information associated with a user in the user profile 118, to perform analytics thereon for recommending treatments (e.g., recommending an optimal dosage of inhibitors) and/or predicting onset of euDKA within a certain time period. The decision support engine 114 may also be used to collect information for pharmaceutical research to develop new therapies or more efficacious therapies. The user profile 118 may be accessible to the decision support engine 114 over one or more networks (not shown) for performing such analytics.

In certain embodiments, the health management system 100 is designed to predict the risk or likelihood of, or the presence and/or severity of, euDKA in real-time (including near real-time) or within a specified period of time for a patient. In certain embodiments, to enable such prediction, the decision support engine 114 is configured to collect information associated with a user in the user profile 118 stored in the user database 110, to perform analytics thereon for: (1) automatically detecting and classifying blood glucose levels and ketone levels; (2) assessing the risk or disease stage of euDKA; (3) assessing the effectiveness of the current treatment and other potential treatment dosages and frequencies; and/or (4) providing an optimal treatment regimen (e.g., dosages and frequencies of consumption) around SGLT-2 inhibitors.

In certain embodiments, the decision support engine 114 may utilize one or more trained machine learning models capable of determining the probability of the presence and/or occurrence of euDKA and/or treatment recommendation for a user based on information provided by user profile 118. In the illustrated embodiment of FIG. 1, the decision support engine 114 may utilize trained machine learning model(s) provided by a training server system 140. Although depicted as a separate server for conceptual clarity, in some embodiments, the training server system 140 and the decision support engine 114 may operate as a single server. That is, the model may be trained and used by a single server (e.g., a local device, a microprocessor, etc.) or may be trained by one or more servers and deployed for use on one or more other servers. In certain embodiments, the model may be trained on one or many virtual machines (VMs) running, at least partially, on one or many physical servers in relational and or non-relational database formats.

The training server system 140 is configured to train the machine learning model(s) using training data, which may include data (e.g., from user profiles) associated with one or more patients (e.g., users or non-users of continuous analyte monitoring system 104 and/or application 106) previously treated for control of blood glucose, as well as patients not treated for control of blood glucose (e.g., healthy patients). The training data may be stored in the historical records database 112 and may be accessible to the training server system 140 over one or more networks (not shown) for training the machine learning model(s). The training data may also, in some cases, include user-specific data for a user over time.

In some embodiments, the training data refers to a dataset that has been featurized and labeled. For example, the dataset may include a plurality of data records, each including information corresponding to a different user profile stored in the user database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model.

As an example, each relevant characteristic of a user, which is reflected in a corresponding data record, may be a feature used in training the machine learning model. Such features may include age, gender, weight, height, body mass index, any therapies currently taken, when a therapy was last applied (e.g., insulin bolus), how much of a therapy was applied (e.g., units of insulin) change (e.g., delta) in analyte levels (e.g., ketone levels, glucose levels) from a first timestamp to a second timestamp, change (e.g., delta) in blood glucose or ketone from a first timestamp to a second timestamp, change (e.g., delta) in analyte thresholds (e.g., ketone thresholds, glucose thresholds) of a user under treatment for blood glucose control from a first timestamp to a subsequent timestamp, the derivative of the measured linear system of analyte measurement (e.g., ketone measurement, glucose measurement) at a point at a specific timestamp, ratio between a glucose measurement and a ketone measurement at a specific timestamp, and/or the difference in derivatives to determine rates of change in the slope of the increase or decrease in analyte values (e.g., ketone values, glucose values), etc. In addition, the data record may be labeled with an indication as to a euDKA diagnosis, an assigned severity, and/or an identified risk of euDKA, prescription information (e.g., dosage and frequency of consumption) for one or more SGLT-2 inhibitors, associated with a patient of the user profile.

The model(s) are then trained by the training server system 140 using the featurized and labeled training data. In particular, the features of each data record may be used as input into the machine learning model(s), and the generated output may be compared to label(s) associated with the corresponding data record. The model(s) may compute a loss based on the difference between the generated output and the provided label(s). This loss is then used to modify the internal parameters or weights of the model. By iteratively processing each data record corresponding to each historical patient, in certain embodiments, the model(s) may be iteratively refined, and the loss minimized, to generate, within a prescribed level of confidence, treatment recommendations (e.g., an optimal dosage/frequency of taking inhibitors) and predictions associated with euDKA risk, presence, progression, improvement (e.g., regression), and severity in a patient. Further, in certain other embodiments, by iteratively processing each data record corresponding to each historical patient, in certain embodiments, the model(s) may be iteratively refined to generate accurate treatment recommendations and predictions of the risk and/or presence of euDKA. Note that although certain embodiments herein are described in relation to providing treatment recommendations for reducing the risk of euDKA and predictions of the risk and/or presence of euDKA, the embodiments described herein are similarly applicable to providing treatment recommendations for reducing the risk of any type of ketoacidosis and also providing predictions of the risk and/or presence of any type of ketoacidosis.

As illustrated in FIG. 1, the training server system 140 deploys these trained model(s) to the decision support engine 114 for use during runtime. For example, the decision support engine 114 may obtain the user profile 118 associated with a user, use information in the user profile 118 as input into the trained model(s), and output a treatment recommendation and/or euDKA prediction. The treatment recommendation may be indicative of an efficacy of a current treatment based on the medication info 124, the analyte data, and the like. In some embodiments, the treatment recommendation includes a modification to an existing treatment or a recommendation for an alternative treatment based on the efficacy of a current treatment. For example, the treatment recommendation may include a change to a current dosage and/or frequency, a change in medication type, or a notice to consult a healthcare provider, etc.

The decision support engine 114 may provide a prediction which may be indicative of the presence and/or severity of euDKA for the user in real-time or within a certain time (e.g., shown as the output 144 in FIG. 1). The output 144 generated by the decision support engine 114 may also provide one or more recommendations for treatment based on the predictions. The output 144 may be provided to the user (e.g., through application 106), to a user's caretaker (e.g., a parent, a relative, a guardian, a teacher, a nurse, etc.), to a user's physician, or any other individual that has an interest in the wellbeing of the user for purposes of improving the user's health, such as, in some cases by effectuating the recommended treatment.

In certain embodiments, the user's own data is used to personalize the one or more models that are initially trained based on population data. For example, a model (e.g., trained using population data) may be deployed for use by decision support engine 114 to provide a treatment recommendation and/or predict the presence and/or severity of euDKA for a specific user. In some embodiments, sometime after making a prediction using the model, the decision support engine 114 may be configured to ask the user, or a caretaker, physician, etc., whether the medication info 124 should be updated based on the recommended change in treatment. In other embodiments, the decision support engine 114 may provide a query as to whether the predicted presence and/or severity of euDKA was confirmed by, e.g., other diagnostic methods (e.g., fingerstick test strips for blood ketones, breath ketone measurement, test strips for urinary ketones), and/or decision support engine 114 may use one or more diagnostic tests to confirm the diagnosis. In some cases, the user's answer and/or results from the diagnostic test(s) performed may deny the presence of euDKA. Accordingly, the model may continue to be retrained and/or personalized using updated medication info 124, the user's answer, the user's test results, and/or physiological parameters of the user. While specific examples are given, other data may also be used as input into the model to personalize the model for the user.

In certain embodiments, the output 144 generated by the decision support engine 114 may be stored in the user profile 118. In certain embodiments, the output 144 may be patient-specific treatment recommendations, treatment efficacy, identification of one or more indicators of euDKA, and the like. For example, in certain embodiments, the output 144 may be a treatment recommendation for an update in medication, medication dosage, medication frequency of use, prediction as to the presence and/or severity of euDKA in a user, and the like. In certain embodiments, the output 144 may be a prediction as to the risk of the onset of euDKA. In certain embodiments, the output 144 may be a prediction as to the risk of a user having hyperglycemia and/or hypoglycemia. In certain embodiments, the output 144 may be a prediction as to a mortality risk of the patient. In certain embodiments, the output 144 may be patient-specific treatment decisions or recommendations for glucose control for the patient. In specific embodiments, the output 144 may be a recommendation relating to the use of an inhibitor (e.g., SGLT2), a recommendation relating to the use of insulin, etc.

In some embodiments, the output 144 stored in the user profile 118 may be continuously updated by the decision support engine 114. Accordingly, previous diagnoses and/or physiological parameters of the user associated with blood glucose control, originally stored as the outputs 144 in the user profile 118 in the user database 110 and then passed to the historical records database 112, may provide an indication of the effectiveness of the current treatment or may provide a likelihood of onset of euDKA in a user in a given time period. Additionally, previous diagnoses and/or physiological parameters of the user associated with how well medication was tolerated and/or how efficacious a certain type/dose/frequency of administration of the medication was, originally stored as the outputs 144 in the user profile 118 in the user database 110 and then passed to the historical records database 112, may provide an indication of the effectiveness of the current treatment or may provide a likelihood of onset of euDKA in a user in a given time period.

In certain embodiments, a user's own historical data may be used to provide decision support and insight around the user's blood glucose control and/or condition onset. For example, a user's historical data may be used by an algorithm as a baseline to indicate improvements or deterioration in the user's condition. As an illustrative example, a user's data from two weeks prior may be used as a baseline that can be compared with the user's current data to identify an improvement or deterioration in glucose and/or ketone levels of the user and, thereby, whether the risk associated with a future euDKA event has increased or decreased. In certain embodiments, the user's own historical data may be used by the training server system 140 to train a personalized model that may further be able to predict the presence and/or severity of euDKA, optimal treatments for reducing the predicted presence and/or severity of euDKA, and/or improvement/deterioration in the user's ketone and/or glucose data based on the user's recent pattern of data (e.g., exercise data, food consumption data, etc.).

In certain embodiments, the model may be trained to provide lifestyle recommendations, exercise recommendations, food intake recommendations, and other types of decision support recommendations to help the user improve treatment or prevent onset and/or progression of euDKA based on the user's historical data, including how different types of medication, food, and treatment (e.g., medication type, dosage, frequency) have impacted the user's analyte levels in the past. In certain embodiments, the model may be trained to predict the underlying cause of certain improvements or deteriorations in the patient's analyte levels. For example, the application 106 may display a user interface with a graph that shows the patient's analyte levels or a score thereof with trend lines and indicate, e.g., retrospectively, what caused the change in analyte levels at certain points in time (e.g., administration of insulin, administration of SGLT2, etc.).

FIG. 2 is a diagram 200 conceptually illustrating an example continuous analyte monitoring system 104 including an example continuous analyte sensor(s) with sensor electronics, in accordance with certain aspects of the present disclosure. For example, the system 104 may be configured to continuously monitor one or more analytes of a user, in accordance with certain aspects of the present disclosure.

Generally, real-time or continuous measurements of analyte levels, rates of change, trends, clearance rates, and/or other analyte data, as measured in interstitial fluid or blood by a continuous analyte monitoring system, can be used to provide treatment recommendations to the user. Such data can indicate a change in analyte levels indicative of a treatment that is less than ideal. Therefore, continuous analyte monitoring may provide earlier, and/or improved treatment recommendations, such as improving the titration of pharmacologic agents with narrow therapeutic windows or evolving pharmacokinetic profiles. Some embodiments may provide screening, diagnosis, prognosis, and/or staging of euDKA as compared to conventional diagnostics.

In certain embodiments, clinical indicators may be used to determine whether a continuous analyte monitoring system, e.g., the continuous analyte monitoring system 104, may be needed to assess an efficacy of a treatment or to assess a risk, likelihood, presence, and/or stage of euDKA in a patient. In one example, such clinical indicators include glucose measurements, ketone measurements, lactate measurements, dissolved oxygen measurements, ion measurements, blood pressure measurements, renal metrics, hydration measurements, physical activity metrics, sleep metrics, heart rate, respiration rate, core temperature, nutrition information, etc. Analytes and other information may generally indicate a needed optimization of a medication dosage and/or frequency or may indicate the presence, risk, or likelihood of euDKA.

In yet another example, clinical indicators may include prescribed or taken medications. For a patient taking certain medications associated with blood glucose control, or medication associated with increased likelihood of euDKA, it may be desirable to monitor for euDKA. For example, a patient on a medication known to be a factor in the occurrence of euDKA may use a continuous analyte monitor to optimize the medication or predict euDKA, which may have been, at least partially, caused by the medication.

In yet another example, clinical indicators may include an assessment of patient adherence to treatment. A comparison of a prescribed treatment to an actual treatment to quantify how well a patient is complying with the prescribed treatment may allow for a more accurate assessment of the efficacy of the prescribed treatment. The comparison may be useful to healthcare providers to further adjust treatment or provide additional treatment instruction/education to the patient. The comparison may also be of interest to healthcare insurers and/or healthcare payers with respect to reimbursement or other considerations (e.g., rewards, discounts, etc.).

In yet another example, clinical indicators may include comorbidities often associated with, and/or increasing the risk of, euDKA. Comorbidities associated with euDKA may include cardiovascular disease, chronic kidney disease, liver disease (e.g., NAFLD, NASH), obesity, activity level, diet, hypertension, hyperlipidemia, etc.

In certain embodiments, continuous analyte monitoring system 104 may be utilized as a short-term diagnostic tool to monitor a new or updated medication type, dosage, and/or frequency for patient reaction, assessing how well the medication is tolerated, and any negative results (e.g., euDKA). For example, a triggering action (e.g., triggering while wearing an analyte sensor), may indicate utility for a patient to wear an analyte sensor (continuous or non-continuous) for a short time period to provide monitoring for patient response to inhibitors and/or for monitoring for euDKA in the patient. In one instance, a patient may wear a short-term continuous or non-continuous analyte sensor for a given period after starting a new medication regimen. In another instance, a patient may wear a short-term continuous or non-continuous analyte sensor periodically (e.g., every 4 weeks) to monitor efficacy of the new medication and provide recommendations for further changes or optimizations to the treatment. In yet another instance, a patient may wear a short-term continuous or non-continuous analyte sensor to predict a likelihood of euDKA. In certain scenarios, data from the analyte sensor can be presented directly to the user. In other diagnostic scenarios, the analyte sensor can be operated in data logging mode, thereby blinding the user to analyte data while allowing the physician to review said data at a later time.

In some embodiments, the continuous analyte monitoring system 104 may be utilized as a long-term diagnostic tool (i.e., greater than 14 days) to monitor patient response and further update the treatment recommendations. Monitoring the patient may also be useful in ongoing prediction and alerts for onset of euDKA. For example, a patient at high risk for euDKA and/or adverse events may utilize a continuous analyte sensor to continually provide insight into the efficacy of the currently prescribed treatment, recommend further updates to the treatment, or predict euDKA for days, weeks, months, etc.

As shown in FIG. 2, the continuous analyte monitoring system 104 in the illustrated embodiment includes a sensor electronics module 204 and one or more continuous analyte sensor(s) 202 (individually referred to herein as the continuous analyte sensor 202 and collectively referred to herein as the continuous analyte sensors 202) associated with a sensor electronics module 204. The sensor electronics module 204 may be in wireless communication (e.g., directly or indirectly) with one or more display devices 210, 220, 230, and 240. In certain embodiments, the sensor electronics module 204 may also be in wireless communication (e.g., directly or indirectly) with one or more medical devices 208 (individually referred to herein as the medical device 208 and collectively referred to herein as the medical devices 208), and/or one or more other non-analyte sensors 206 (individually referred to herein as the non-analyte sensor 206 and collectively referred to herein as the non-analyte sensor 206). In other embodiments, including, but not limited to, diagnostic implementations, the sensor electronics module may be operated independently (e.g., unpaired with a display device) and queried at the end of a wear session to wirelessly transfer data logged during a session to a local device or Cloud database for future review, retrieval, or execution of further analytics.

In certain embodiments, a continuous analyte sensor 202 may comprise a sensor for detecting and/or measuring analyte(s). The continuous analyte sensor 202 may be a multi-analyte sensor configured to continuously measure two or more analytes (e.g., ketone, glucose) or a single analyte sensor configured to continuously measure a single analyte as a non-invasive device, a subcutaneous device, a transcutaneous device, a transdermal device, a dermal device, an intradermal device, a subdermal device, implanted device, and/or an intravascular device. In certain embodiments, the continuous analyte sensor 202 may be configured to continuously measure analyte levels of a user using one or more measurement techniques, such as enzymatic, immunometric, aptameric, amperometric, voltametric, potentiometric, impedimetric, conductimetric, conductometric, capacitive, chemical, physical, electrochemical, spectrophotometric, polarimetric, calorimetric, iontophoretic, radiometric, immunochemical, optical, ion-selective and the like. In certain aspects, the continuous analyte sensor 202 provides a data stream indicative of the concentration of one or more analytes in the user. The data stream may include raw data signals which may be converted into a calibrated and/or filtered data stream used to provide estimated analyte value(s) to the user.

In certain embodiments, the continuous analyte sensor 202 may be a multi-analyte sensor configured to continuously measure multiple analytes in a user's body. For example, in certain embodiments, the continuous multi-analyte sensor 202 may be a single sensor configured to measure glucose, ketones, and/or other analytes circulating in the user's body.

In certain embodiments, one or more multi-analyte sensors may be used in combination with one or more single analyte sensors. As an illustrative example, a multi-analyte sensor may be configured to continuously measure ketone and glucose and may, in some cases, be used in combination with one or more other analyte sensors configured to measure only, for example, lactate levels, oxygen levels, hydration levels or hormone levels. In various embodiments, a multi-analyte sensor can include a single body-worn wearable or two distinct body-worn wearables. Information from each of the multi-analyte sensor(s) and single analyte sensor(s) may be combined to provide treatment decision support using methods described herein.

In certain embodiments, the sensor electronics module 204 includes electronic circuitry associated with measuring and processing the continuous analyte sensor data, including prospective algorithms associated with processing and calibration of the sensor data. The sensor electronics module 204 can be physically connected to the continuous analyte sensor(s) 202 and can be integral with (non-releasably attached to) or releasably attachable to the continuous analyte sensor(s) 202. The sensor electronics module 204 may include hardware, firmware, and/or software that enables measurement of levels of analyte(s) via a continuous analyte sensor(s) 202. For example, the sensor electronics module 204 can include an electrochemical analog front end (e.g., potentiostat, galvanostat, impedance measurement device), a power source for providing power to the sensor, a microprocessor for executing an embedded data processing or algorithmic routine, other components useful for signal processing and data storage, and a telemetry module for transmitting data from the sensor electronics module to one or more display devices or a centralized data repository. Electronics can be affixed to a printed circuit board (PCB), flexible PCB (flexPCB), or the like, and can take a variety of forms. For example, the electronics can take the form of an integrated circuit (IC), such as an Application-Specific Integrated Circuit (ASIC), a field-programmable gate array (FPGA), a system-on-a-chip (SoC), a microcontroller, and/or a processor.

In some embodiments, the display devices 210, 220, 230, and/or 240 are configured for displaying displayable sensor data, including analyte data, which may be transmitted by the sensor electronics module 204. Each of the display devices 210, 220, 230, or 240 can include a display such as a touchscreen display 212, 222, 232, or 242 for displaying sensor data to a user and/or receiving inputs from the user. For example, a graphical user interface (GUI) may be presented to the user for such purposes. In some embodiments, the display devices 210, 220, 230, and 240 may include other types of user interfaces such as a voice user interface instead of, or in addition to, a touchscreen display for communicating sensor data to the user of the display device and/or receiving user inputs. The display devices 210, 220, 230, and 240 may be examples of the display device 107 illustrated in FIG. 1 used to display sensor data to a user of FIG. 1 and/or receive input from the user.

In some embodiments, one, some, or all of the display devices are configured to display or otherwise communicate the sensor data as it is communicated from the sensor electronics module (e.g., in a data package that is transmitted to respective display devices), without any additional prospective processing required for calibration and real-time display of the sensor data.

The plurality of display devices may include a custom display device specially designed for displaying certain types of displayable sensor data associated with analyte data received from sensor electronics module. In certain embodiments, the plurality of display devices may be configured for providing alerts/alarms/notifications based on the displayable sensor data. The display device 210 is an example of such a custom device. In some embodiments, one of the plurality of display devices is a smartphone, such as the display device 220 which represents a mobile phone, using a commercially available operating system (OS), and configured to display a graphical representation of the continuous sensor data (e.g., including current and historic data). Other display devices can include other hand-held devices, such as the display device 230 which represents a tablet, the display device 240 which represents a smart watch, the medical device 208 (e.g., an insulin delivery device or a blood glucose meter), and/or a desktop or laptop computer (not shown).

Because different display devices provide different user interfaces, the content of the data packages (e.g., amount, format, and/or type of data to be displayed, alarms, and the like) can be customized (e.g., programmed differently by the manufacture and/or by an end user) for each particular display device. Accordingly, in certain embodiments, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor data. In certain embodiments, the type of alarms customized for each particular display device, the number of alarms customized for each particular display device, the timing of alarms customized for each particular display device, and/or the threshold levels configured for each of the alarms (e.g., for triggering) are based on the output 144 (e.g., as mentioned, the output 144 may be indicative of the current health of a user, the state of a user's ketone and/or glucose levels, current treatment recommended to a user, and/or physiological parameters of a user) stored in the user profile 118 for each user.

As mentioned, the sensor electronics module 204 may be in communication with a medical device 208. The medical device 208 may be a passive device in some example embodiments of the disclosure. For example, the medical device 208 may be an insulin pump for administering insulin to a user. For a variety of reasons, it may be desirable for such an insulin pump to receive and track ketone and/or glucose values transmitted from the continuous analyte monitoring system 104, where the continuous analyte sensor 202 is configured to measure ketone and/or glucose.

Further, as mentioned, the sensor electronics module 204 may also be in communication with other non-analyte sensors 206. The non-analyte sensors 206 may include, but are not limited to, an altimeter sensor, an accelerometer sensor, a temperature sensor, a respiration rate sensor, a sweat sensor, etc. The non-analyte sensors 206 may also include monitors such as heart rate monitors, ECG monitors, blood pressure monitors, pulse oximeters, caloric intake, and medicament delivery devices. The non-analyte sensors 206 may also include data systems for measuring non-patient specific phenomena such as time, ambient pressure, or ambient temperature which could include an atmospheric pressure sensor, an external air temperature sensor or a clock, timer, or other time measure of when the sensor was first inserted or a measure of sensor life remaining compared to insertion time could be used as calibration or other data inputs for an algorithmic model. One or more of these non-analyte sensors 206 may provide data to the decision support engine 114 described further below. In some aspects, a user may manually provide some of the data for processing by the training server system 140 and/or the decision support engine 114 of FIG. 1.

In certain embodiments, the non-analyte sensors 206 may be combined in any other configuration, such as, for example, combined with one or more continuous analyte sensors 202. As an illustrative example, a non-analyte sensor, e.g., a glucose sensor, may be combined with a continuous analyte sensor 202 configured to measure ketone to form a ketone/glucose sensor used to transmit sensor data to the sensor electronics module 204 using common communication circuitry.

In certain embodiments, a wireless access point (WAP) may be used to couple one or more of the continuous analyte monitoring system 104, the plurality of display devices, the medical device(s) 208, and/or the non-analyte sensor(s) 206 to one another. For example, the WAP 138 may provide Wi-Fi, cellular, and/or IoT (e.g., NB-IoT, LTE Cat-M1) connectivity among these devices. Near Field Communication (NFC) and/or Bluetooth may also be used among devices depicted in the diagram 200 of FIG. 2.

FIG. 3 illustrates a diagram 300 of example inputs and example metrics that are calculated based on the inputs for use by the health management system of FIG. 1, according to some embodiments disclosed herein. In particular, FIG. 3 provides a more detailed illustration of example inputs and example metrics introduced in FIG. 1.

FIG. 3 shows example inputs 128 on the left, the application 106 and the decision support engine 114 including the DAM 116 in the middle, and metrics 130 on the right. In certain embodiments, each one of the metrics 130 may correspond to one or more values, e.g., discrete numerical values, ranges, or qualitative values (high/medium/low, stable/unstable, rate of change, points of inflection, etc.). The application 106 obtains the inputs 128 through one or more channels (e.g., manual user input, sensors/monitors, other applications executing on the display device 107, EMRs, etc.). As mentioned previously, in certain embodiments, the inputs 128 may be processed by the DAM 116 and/or the decision support engine 114 to output the metrics 130. The inputs and metrics 130 may be used by the decision support engine 114 to provide decision support to the user. For example, the inputs 128 and the metrics 130 may be used by the training server system 140 to train and deploy one or more machine learning models for use by the decision support engine 114 for providing decision support around treatment of the patient.

In certain embodiments, starting with the inputs 128, food consumption information may include information about one or more of meals, snacks, and/or beverages, such as one or more of the size, content (milligrams (mg) of sodium, potassium, carbohydrate, fat, protein, etc.), sequence of consumption, and time of consumption. In certain embodiments, food consumption may be provided by a user through manual entry, by providing a photograph through an application that is configured to recognize food types and quantities, by scanning a bar code or menu, and/or interrogating an NFC/RFID tag integrated into the packaging of the food item. In various examples, meal size may be manually entered as one or more of calories, quantity (e.g., “three cookies”), menu items (e.g., “Royale with Cheese”), and/or food exchanges (e.g., 1 fruit, 1 dairy). In some examples, meal information may be received via a convenient user interface provided by the application 106. In some examples, meal information may be provided via one or more other applications synchronized with the application 106, such as one or more other mobile health applications executed by the display device 107. In such examples, the synchronized applications may include, e.g., an electronic food diary application or photograph application.

In certain embodiments, food consumption information entered by a user may relate to nutrients consumed by the user. Consumption may include any natural or designed food or beverage. Food consumption information entered by a user may also be related to analytes, including any of the other analytes described herein.

In certain embodiments, exercise information is also provided as an input. Exercise information may be any information surrounding activities, such as activities requiring physical exertion by the user. For example, exercise information may range from information related to low intensity (e.g., walking a few steps) and high intensity (e.g., five mile run) physical exertion or it could take the form of a wattage (e.g., stationary cycle), speed (e.g., GPS-enabled smartwatch), and/or resistance (e.g., elliptical machine) over a specified time interval. In certain embodiments, exercise information may be provided, for example, by an accelerometer sensor or a heart rate monitor on a wearable device such as a watch, fitness tracker, and/or patch. In certain embodiments, exercise information may also be provided through manual user input, through workout machinery, and/or through a surrogate sensor and prediction algorithm measuring changes to heart rate (or other cardiac metrics). When predicting that a user is exercising based on his/her sensor data, the user may be asked to confirm if exercise is occurring, what type of exercise, and or the level of strenuous exertion being used during the exercise over a specific period. This data may be used to train the system to learn about the user's exercise patterns to reduce the need for confirmation questions as time progresses. Other analytes and sensor data may also be included in this training set, including analytes and other measured elements described herein including temporal elements, such as time and day.

In certain embodiments, user statistics, such as one or more of age, height, weight, BMI, body composition (e.g., % body fat), stature, build, or other information may also be provided as an input. In certain embodiments, user statistics are provided through a user interface, by interfacing with an electronic source such as an electronic medical record, and/or from measurement devices. In certain embodiments, the measurement devices include one or more of a wireless, e.g., Bluetooth-enabled, weight scale and/or camera, which may, for example, communicate with the display device 107 to provide user data.

In certain embodiments, treatment information is also provided as an input. Treatment information may include information about the type, dosage, and/or timing of when one or more medications (e.g., SGLT2, insulin, glucagon, sulfonylurea, metformin, GLP-1) are to be taken by the user. As mentioned herein, the treatment information may include information about one or more inhibitors, one or more drugs known to reduce blood glucose levels, one or more drugs known to affect ketone, and/or one or more medications for treating one or more symptoms of acute or chronic conditions and diseases the user may have. The treatment information may include information regarding different lifestyle habits, surgical procedures, and/or other non-invasive procedures recommended by the user's physician. For example, the user's physician may recommend a user increase/decrease their carbohydrate intake, exercise for a minimum of thirty minutes a day, or increase an insulin dosage or other medication to maintain, improve, and/or reduce hyper- and/or hypoglycemic episodes, etc. As another example, a healthcare professional may recommend that a user engage in at-home treatment and/or treatment at a clinic. The treatment information may also indicate a patient's adherence to the prescribed type, dosage, and/or timing of medications. For example, the treatment/medication information may indicate whether and when exactly and with what dosage/type the medication was taken.

In certain embodiments, analyte sensor data may also be provided as input, for example, through the continuous analyte monitoring system 104. In certain embodiments, analyte sensor data may include ketone data (e.g., a user's ketone values) measured by at least a ketone sensor (or multi-analyte sensor configured to measure at least ketone) that is a part of the continuous analyte monitoring system 104. In certain embodiments, analyte sensor data may include glucose data measured by at least a glucose sensor (or multi-analyte sensor configured to measure at least glucose) that is a part of continuous analyte monitoring system 104.

In certain embodiments, input may also be received from one or more non-analyte sensors, such as the non-analyte sensors 206 described with respect to FIG. 2. Input from the non-analyte sensors 206 may include information related to a heart rate, heart rate variability (e.g., the variance in time between the beats of the heart), ECG data, a respiration rate, oxygen saturation, a blood pressure, or a body temperature (e.g. to detect illness, physical activity, etc.) of a user. In certain embodiments, electromagnetic sensors may also detect low-power radio frequency (RF) fields emitted from objects or tools touching or near the object, which may provide information about user activity or location.

In some embodiments, the non-analyte sensors 206 may include an embedded scanner/reader to detect medication related information (e.g., type, brand, dosage, frequency). Examples of a scanner may include a reader configured to detect near-field communication (NFC) and/or radio frequency identification (RFID) information provided by a corresponding active or passive tag provided within the medication packaging or otherwise accompanying the medication. Another example of a scanner may be a barcode, QR, or other optical scanner capable of accessing information associated with a visual pattern provided on the packaging or otherwise associated with the medication.

In certain embodiments, input received from non-analyte sensors may include input relating to a user's insulin delivery. In particular, input related to the user's insulin delivery may be received, via a wireless connection on a smart pen, via user input, and/or from an insulin pump. Insulin delivery information may include one or more of insulin manufacturer, insulin dosage, insulin formulation, insulin volume, basal vs bolus dose, intended pharmacokinetic profile (e.g., short-acting, long-acting), number of units of insulin delivered, time of delivery, etc. Other parameters, such as insulin action time or duration of insulin action, may also be received as inputs.

In certain embodiments, time may also be provided as an input, such as time of day, UTC time or time from a real-time clock. Said real-time clock may be provided externally (synchronized to a server via a WiFi, cellular, or Bluetooth wireless connection) or may be embedded as an integrated real-time clock (RTC) circuit within the wearable/sensor electronics. For example, in certain embodiments, input analyte data may be timestamped to indicate a date and time when the analyte measurement was taken for the user.

User input of any of the above-mentioned inputs 128 may be through a user interface, such a user interface of the display device 107 of FIG. 1.

As described above, in certain embodiments, the DAM 116 and/or the decision support engine (e.g., using one or more trained models) determines or computes the user's metrics 130 based on the inputs 128. An example list of the metrics 130 is shown in FIG. 3.

In certain embodiments, ketone and/or glucose levels may be determined from sensor data. For example, ketone levels refer to time-stamped ketone measurements or values that are continuously generated and stored over time.

In certain other embodiments, the DAM 116 may use ketone and/or glucose levels measured over a period of time where the user is (i.e., GPS coordinates), at least for a subset of the period of time, engaging in exercise and/or consuming nutrients and/or an external condition exists that would affect the ketone/glucose levels. In such embodiments, the DAM 116 may, in some examples, first identify which measured analyte values are not to be used for calculating the baseline by identifying which analyte values have been affected by an external event, such as the consumption of food, exercise, medication, or other perturbation that would disrupt the capture of an analyte baseline measurement. The DAM 116 may then exclude such measurements when calculating the analyte baseline of the user. In some other examples, the DAM 116 may calculate the analyte baseline by first determining a percentage of the number of analyte values measured during this time period that represent the lowest analyte values measured. The DAM 116 may then take an average of such analyte values to determine the analyte baseline level.

In certain embodiments, an absolute maximum analyte level (e.g., ketone, glucose) may be determined from sensor data, health/sickness metrics (e.g., described in more detail below), and/or other condition metrics. The absolute maximum analyte level represents a user's maximum analyte level determined to be safe over a period of time (e.g., hourly, weekly, daily, etc.). In certain embodiments, the absolute maximum analyte level may be consistent across all users. In certain other embodiments, each patient may have a different absolute maximum analyte level. In certain embodiments, the absolute maximum analyte level per patient may change over time. For example, a user may be initially assigned an absolute maximum analyte level based on clinical input. This assigned absolute maximum analyte level may be adjusted over time based on other sensor data, comorbidities, etc. for the patient. Minimum analyte values may be determined in a similar manner.

In certain embodiments, analyte thresholds other than an absolute maximum and/or minimum analyte level of a user may be determined from sensor data (e.g., ketone/glucose measurements obtained from a continuous sensor of continuous analyte monitoring system 104), health/sickness metrics (e.g., described in more detail below), and/or other condition metrics. Such analyte thresholds may represent, e.g., the maximum or minimum analyte levels determined to be safe during certain activities, which may vary across different activities. For example, because exercise is known to affect ketone/glucose levels, the maximum and/or minimum ketone/glucose thresholds for a user during exercise may be different than maximum and/or minimum ketone/glucose thresholds for the user during other activities.

In certain embodiments, analyte level rates of change may be determined from the sensor data (e.g., ketone/glucose measurements obtained from the continuous analyte monitoring system 104 over time). For example, a ketone level rate of change refers to a rate that indicates how one or more time-stamped ketone measurements or values change in relation to one or more other time-stamped ketone measurements or values. Ketone level rates of change may be determined over one or more seconds, minutes, hours, days, etc.

In certain embodiments, determined analyte level rates of change may be marked as “increasing rapidly” or “decreasing rapidly”. As used herein, “rapidly” may describe analyte level rates of change that are clinically significant and pointing towards a trend of the analyte levels likely breaching the absolute maximum analyte level or the absolute minimum analyte level within a defined period of time. In other words, a predictive trend (e.g., produced by the decision support engine 114 using one or more trained models) may, in some cases, indicate that a patient is likely to hit, for example, the absolute maximum analyte level within a specified time period (e.g., one or two hours) based on the determined analyte level rate of change. Accordingly, such an analyte level rate of change may be marked as “increasing rapidly”. Similarly, a predictive trend (e.g., produced by the decision support engine 114 using one or more trained models) may, in some cases, indicate that a patient is likely to hit the absolute minimum analyte level within a specified time period (e.g., one or two hours) based on the analyte level rate of change determined. Accordingly, such an analyte level rate of change may be marked as “decreasing rapidly”.

In certain embodiments, analyte (e.g., ketone, glucose) baseline rates of change may be determined from analyte baselines determined for a user over time. For example, a ketone baseline rate of change refers to a rate that indicates how one or more time-stamped ketone baselines for a user change in relation to one or more other time-stamped ketone baselines for the same user. Ketone baseline rates of change may be determined over one or more seconds, minutes, hours, days, etc.

In certain embodiments, an analyte clearance rate may be determined from sensor data (e.g., ketone/glucose measurements obtained from a CKM/CGM of the continuous analyte monitoring system 104) following the consumption of a known, or estimated, amount of analyte. The analyte clearance rates analyzed over time may be indicative of medication efficacy or onset of a condition. In particular, the slope of a curve of analyte clearance during a first time period (e.g., after administration of an inhibitor) compared to the slope of a curve of an analyte clearance during a second time period (e.g., after consuming the same inhibitor) may be indicative of an effectiveness of a treatment.

In certain embodiments, the analyte clearance rate may be determined by calculating a slope between a first value (e.g., during a period of increased levels) at t₀ and the user's analyte baseline reached at t₁. In certain embodiments, an analyte clearance rate may be calculated over time until the increased analyte levels of the user reach some value relative to the user's analyte baseline (e.g., % of a user's analyte baseline). Analyte clearance rates calculated over time may be time-stamped and stored in the user's profile 118.

In certain embodiments, a standard deviation of analyte levels (not shown) may be determined from sensor data. In some examples, a standard deviation of one or more analyte levels may be determined based on the variability of one or more analyte levels as compared to an average analyte level over one or more time periods. In some embodiments, a time-in-range metric (not shown) may be determined from the analyte data. For example, with an established upper limit and lower limit, the time period during which the analyte data was between the upper and lower limits can be determined. The time-in-range may be determined for individual instances of the analyte data being in-range or may be determined over a predetermined length of time (one day) for which each individual in-range periods are summed.

In certain embodiments, analyte trends may be determined based on analyte levels over certain periods of time. In certain embodiments, ketone/glucose trends may be determined based on ketone/glucose baselines over certain periods of time. In certain embodiments, analyte trends may be determined based on absolute analyte level minimums over certain periods of time. In certain embodiments, analyte trends may be determined based on absolute maximum analyte levels over certain periods of time. In certain embodiments, analyte trends may be determined based on analyte level rates of change over certain periods of time. In certain embodiments, analyte trends may be determined based on analyte baseline rates of change over certain periods of time. In certain embodiments, analyte trends may be determined based on calculated analyte clearance rates over certain periods of time.

In certain embodiments, glucose levels may be determined from sensor data (e.g., glucose measurements obtained from a continuous glucose sensor of continuous analyte monitoring system 104).

In certain embodiments, glucose level rates of change may be determined from sensor data (e.g., glucose measurements obtained from a continuous glucose monitor (CGM) of continuous analyte monitoring system 104 over time). For example, a glucose level rate of change refers to a rate that indicates how one or more time-stamped glucose measurements or values change in relation to one or more other time-stamped glucose measurements or values. Glucose level rates of change may be determined over one or more seconds, minutes, hours, days, etc.

In certain embodiments, a glucose trend may be determined based on glucose levels over a certain period of time. In certain embodiments, glucose trends may be determined based on glucose level rates of change over certain periods of time.

In certain embodiments, glycemic variability may be determined from sensor data (e.g., glucose measurements obtained from continuous analyte monitoring system 104 over time). For example, glycemic variability refers to a standard deviation of glucose levels over a period of time. Glycemic variability may be determined over one or more minutes, hours, days, etc.

In certain embodiments, a glucose clearance rate may be determined from sensor data (e.g., glucose levels obtained from a continuous glucose sensor of the continuous analyte monitoring system 104) following the consumption of a known, or estimated, amount of glucose or know nutrient resulting in production of glucose. Glucose clearance rates analyzed over time may be indicative of glucose homeostasis. Glucose trends may be indicative of an effectiveness of a medication type, dosage, and/or frequency.

In certain embodiments, the glucose clearance rate may be determined by calculating a slope between an initial high glucose value (e.g., highest glucose level during a period of 20-30 minutes after the consumption of glucose) at t₀ and a subsequent low glucose value at t₁. The low glucose value (G_{L}) may be determined based on a user's initial high glucose value (G_{H}) and baseline glucose value (G_{B}) before the consumption of glucose. In certain embodiments, G_{L} can be a glucose value between G_{H} and G_{B}, e.g., G_{L}=G_{B}+K*(G_{H}−G_{B})/2, where K can be a percentage representing by how much a user's glucose level returned to the user's baseline value. When K equals zero, the low glucose value equals the baseline glucose value. When K equals 0.5, the low glucose value equals the mean glucose value between the initial glucose value and the baseline glucose value.

In certain embodiments, the glucose clearance rate may be determined over one or more periods of time after the consumption of glucose, such as following an oral glucose tolerance test (OGTT). The glucose clearance rate may be calculated for each time period to represent the dynamics of glucose clearance rate after the consumption of glucose. These glucose clearance rates calculated over time may be time-stamped and stored in the user's profile 118. Certain metrics may be derived from the time-stamped glucose clearance rates, such as mean, median, standard deviation, percentile, etc.

In certain embodiments, insulin sensitivity may be determined using historical data, real-time data, or a combination thereof, and may, for example, be based upon one or more inputs 128, such as one or more of food consumption information, continuous analyte sensor data, non-analyte sensor data (e.g., insulin delivery information from an insulin device), etc. Insulin sensitivity refers to how responsive a user's cells are to insulin. Improving insulin sensitivity for a user may help to reduce insulin resistance in the user.

In certain embodiments, insulin on board may be determined using non-analyte sensor data input (e.g., insulin delivery information) and/or known or learned (e.g. from user data) insulin time action profiles, which may account for both basal metabolic rate (e.g., update of insulin to maintain operation of the body) and insulin usage driven by activity or food consumption.

In certain embodiments, an insulin clearance rate may be determined using historical data, real-time data, or a combination thereof, e.g., by calculating a slope between an initial insulin value (e.g., during a period of increased insulin levels) at t₀ and a final insulin value of the user at t₁. Ketone metrics may be similarly monitored and determined.

In certain embodiments, health and sickness metrics may be determined, for example, based on one or more of user input (e.g., pregnancy information or known sickness or disease information), from physiologic sensors (e.g., temperature), activity sensors, or a combination thereof. In certain embodiments, based on the values of the health and sickness metrics, for example, a user's state may be defined as being one or more of healthy, ill, rested, or exhausted.

In certain embodiments, the meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example, from food consumption information, time of meal information, and/or digestive rate information, which may be correlated to food type, quantity, and/or sequence (e.g., which food/beverage was eaten first).

In certain embodiments, meal habits metrics are based on the content and the timing of a user's meals. For example, if a meal habit metric is on a scale of 0 to 1, the better/healthier meals the user eats the higher the meal habit metric of the user will be to 1, in an example. Also, the more the user's food consumption adheres to a certain time schedule or a recommended diet, the closer their meal habit metric will be to 1, in the example.

In certain embodiments, medication adherence (not shown) is measured by one or more metrics that are indicative of how committed the user is towards their medication regimen. In certain embodiments, medication adherence metrics are calculated based on one or more of the timing of when the user takes medication (e.g., whether the user is on time or on schedule), the type of medication (e.g., is the user taking the right type of medication), and the dosage of the medication (e.g., is the user taking the right dosage). In certain embodiments, medication adherence of a user may be determined in a clinical trial where medication consumption and timing of such medication consumption is monitored, through user input, and/or based on analyte data received from the continuous analyte monitoring system 104.

In certain embodiments, the activity level metric may indicate the user's level of activity. In certain embodiments, the activity level metric may be determined, for example, based on input from an activity sensor or other physiologic sensors, such as the non-analyte sensors 206. In certain embodiments, the activity level metric may be calculated by the DAM 116 based on one or more of the inputs 128, such as one or more of exercise information, non-analyte sensor data (e.g., accelerometer data), time, user input, etc. In certain embodiments, the activity level may be expressed as a step rate of the user. Activity level metrics may be time-stamped so that they can be correlated with the user's lactate levels at the same time.

In certain embodiments, exercise regimen metrics (not shown) may indicate one or more of the type of activities the user engages in, the corresponding intensity of such activities, frequency the user engages in such activities, etc. In certain embodiments, exercise regimen metrics may be calculated based on one or more of the analyte sensor data input (e.g., from a lactate monitor, a glucose monitor, etc.), non-analyte sensor data input (e.g., non-analyte sensor data input from an accelerometer, a heart rate monitor, a respiration rate sensor, etc.), calendar input, user input, etc.

In certain embodiments, body temperature metrics may be calculated by the DAM 116 based on the inputs 128, and more specifically, non-analyte sensor data from a temperature sensor. In certain embodiments, the heart rate metrics (e.g., including heart rate and heart rate variability) may be calculated by the DAM 116 based on the inputs 128, and more specifically, non-analyte sensor data from a heart rate sensor. In certain embodiments, respiratory metrics (not shown) may be calculated by the DAM 113 based on the inputs 128, and more specifically, non-analyte sensor data from a respiratory rate sensor. In certain embodiments, blood pressure metrics (e.g., including blood pressure levels and blood pressure trends) may be calculated by the DAM 113 based on the inputs 128, and more specifically, non-analyte sensor data from the blood pressure sensor.

In certain embodiments, as described in more detail below, physiological parameters (e.g., ketone levels, ketone level rates of change, glucose levels, heart rate, blood pressure, etc.) associated with the user may be stored as metrics 130 when a state or condition of the user is confirmed. In certain embodiments, such physiological parameters may be analyzed over time to provide an indication of changes in the state or condition of the user. In certain embodiments, the user specific values of the physiological parameters experienced by the user may be a valuable input for training one or models designed to assess the current treatment of the user and a likelihood of euDKA in a user. In certain embodiments, the user specific values of the physiological parameters experienced by the user may be used to create one or more personalized models specific to the user for greater accuracy.

### Example Methods and Systems for Providing Decision Support to a Patient

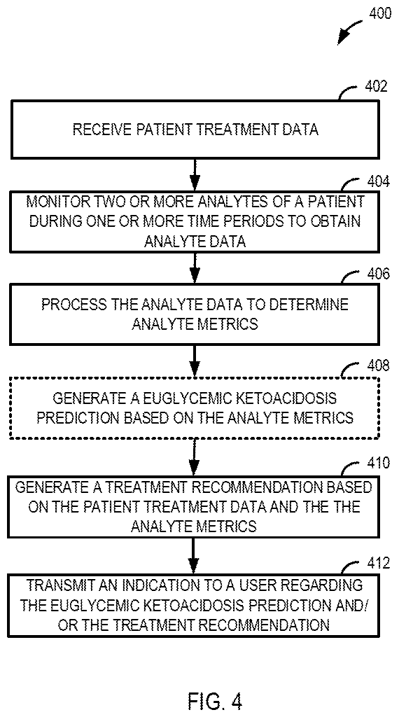

FIG. 4 is a flow diagram illustrating an example method 400 for providing decision support based on patient treatment data. Method 400 may be performed by the health management system 100 to collect/generate data such as inputs 128 and metrics 130, including for example, analyte data, patient information, and non-analyte sensor data mentioned above. For example, the method 400 may be performed by decision support engine 114 to provide decision support to a user using a continuous analyte monitoring system 104 including, at least, a continuous analyte sensor 202, as illustrated in FIGS. 1 and 2. Method 400 is described below with reference to FIGS. 1 and 2 and their components. The method 400 may provide decision support in real-time or within a specified period of time. Generally, real-time or continuous measurements of analyte levels, rates of change, trends, clearance rates, and/or other analyte data, as measured in interstitial fluid or blood, can be used to determine a condition of the user (e.g., medication efficacy, euDKA onset). Therefore, continuous analyte monitoring of analytes, such as ketone and glucose, may provide improved treatment recommendations and/or improved determination of euDKA as compared to conventional diagnostics.

In certain embodiments, the decision support engine 114 of the health management system 100 may use various algorithms or artificial intelligence (AI) models, such as machine-learning models, trained based on patient-specific data and/or population data to provide treatment recommendations and/or euDKA predictions. The algorithms and/or machine-learning models may take into account one or more inputs 128 and/or metrics 130 described with respect to FIG. 3 for a patient.

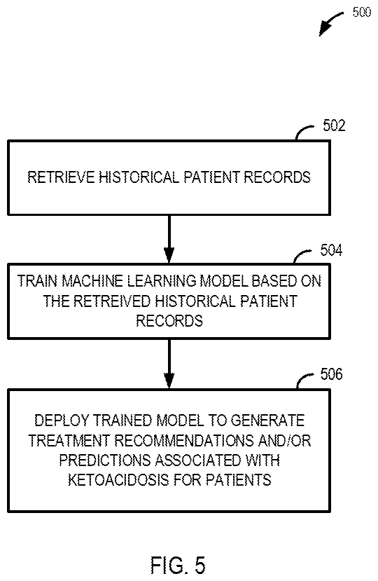

The one or more machine-learning models described herein for making such predictions may be at least initially trained using population data. A method for training the one or more machine learning models may be described in more detail below with respect to FIG. 5.

In certain embodiments, as an alternative to using machine learning models, decision support engine 114 may use rule-based models to provide treatment recommendations and/or predict the risk or likelihood of a patient experiencing euDKA. Rule-based models involve using a set of rules for analyzing data. These rules are sometimes referred to as ‘If statements’ as they tend to follow the line of ‘If X happens, then do or conclude Y’. In particular, decision support engine 114 may apply rule-statements (e.g., if, then statements, do-while statements, catch statements, switch statements, finite state machine framework) to generate the treatment recommendations and/or determine the risk or likelihood of a patient developing euDKA.

Such rules may be defined and maintained by decision support engine 114 in a reference library. For example, the reference library may maintain ranges of analyte (e.g., potassium) levels and ranges of analyte level rates of change (and/or other analyte data) and/or other analyte metrics. In certain embodiments, such rules may be determined based on empirical research or an analysis of historical patient records, such as the records stored in the historical records database 112. In some cases, the reference library may become very granular. For example, other factors may be used in the reference library to create such “rules”. Other factors may include gender, age, diet, medical history, family medical history, body mass index (BMI), etc. Increased granularity may provide more accurate outputs.

At block 402, the method 400 may begin by receiving patient treatment data. This data may be used as a starting point for generating a euDKA prediction and generating a treatment recommendation at block 410. For example, the treatment data may be retrieved from the user profile 118. As described above, treatment information may include information about the type, dosage, and/or timing of when one or more medications (e.g., SGLT2, insulin) are to be taken by the user. As mentioned herein, the treatment information may include information about one or more inhibitors, one or more drugs known to reduce blood glucose levels, one or more drugs known to affect ketone, and/or one or more medications for treating one or more symptoms of acute or chronic conditions and diseases the user may have (e.g., diabetes, cardiovascular disease, kidney disease). The treatment information may also indicate a patient's adherence to the prescribed type, dosage, and/or timing of medications. For example, the treatment/medication information may indicate whether and when exactly and with what dosage/type the medication was taken.

At block 404, the method 400 continues by monitoring one or more analytes of a patient, such as user 102 illustrated in FIG. 1, during one or more time periods (e.g., a plurality of time periods) to obtain analyte data. The one or more analytes monitored may, in certain embodiments, include at least ketone and glucose. Thus, the analyte data may at least contain ketone and glucose data. Block 402 may be performed by the continuous analyte monitoring system 104 illustrated in FIGS. 1 and 2, and more specifically, continuous analyte sensor(s) 202 illustrated in FIG. 2, in certain embodiments. For example, continuous analyte monitoring system 104 may in certain embodiments comprise a continuous analyte monitor 202 configured to measure the patient's analyte levels (e.g., ketone, glucose).

Accordingly, in certain embodiments, the continuous analyte monitoring sensor 202 may collect analyte measurements that can be utilized to generate analyte data including analyte baselines, analyte rates of change, analyte baseline rates of change, personalized analyte levels, average analyte levels, maximum and/or minimum analyte levels, absolute maximum and/or minimum analyte levels, standard deviation of analyte levels, analyte clearance rates, analyte trends, etc.

In certain embodiments, using data for multiple analytes in combination, including data for ketone and glucose, may help to further inform the treatment recommendation and prediction of euDKA, as compared to data for a single analyte. For example, monitoring additional types of analytes in addition to ketone and glucose, as measured by the continuous analyte monitoring system 104, may provide additional insight as compared to insight derived from ketone alone. Some examples of additional types of analytes include, but are not limited to, lactate, uric acid, ethanol, ascorbic acid, creatinine, glutamate, glutathione, sodium, potassium, chloride, cortisol, and insulin. Such additional insight may include indications of other health conditions that may affect efficacy of a medication, such as an inhibitor.

The additional insight gained from using a combination of analytes may increase the accuracy of the treatment recommendation and/or the euDKA prediction. For example, the probability of accurately recommending a treatment or change in treatment, or accuracy in predicting euDKA, may be a function of a number of analytes measured for a patient. For example, in some examples, a probability of accurately predicting that a patient has or will likely develop euDKA while on a particular treatment regimen using only ketone data (in addition to other non-analyte data) may be less than a probability of accurately performing the same using ketone and glucose data (in addition to other non-analyte data), which may also be less than a probability of accurately performing the analysis using ketone, glucose, and lactate data (in addition to other non-analyte data) for analysis.

Further, using a combination of analytes enables the determination of various ratios associated with the analytes (e.g., a ketone-to-glucose ratio), which can further inform the analysis. Such ratios may be determined based on measured analyte values, analyte thresholds, analyte rates of change, analyte variance, analyte clearance rates, and/or any other analyte data associated with the combination of analytes.

Accordingly, in certain embodiments described herein, analyte combinations, e.g., measured and collected by one (e.g., multi-analyte) or more sensors for treatment recommendation and/or predictions of euDKA, include at least ketone and glucose; however, other analyte combinations may also be considered.

In certain embodiments, at block 404, continuous analyte monitoring system 104 may continuously monitor ketone and glucose levels of a patient during a plurality of time periods. In certain embodiments, the measured ketone concentrations may be used in conjunction with glucose levels for determining a treatment recommendation or predicting the likelihood of euDKA. Glucose is a simple sugar (e.g., a monosaccharide). Glucose can be both ingested, as well as, produced in the body from protein, fat, and carbohydrates. Increasing glucose stimulates insulin release. Insulin causes the cells to take in glucose for fuel. Thus, insulin stimulates glucose uptake by cells, thereby reducing glucose levels. In some cases, where glucose levels of a patient are increased and rate(s) of change of glucose levels in the patient's body are high, excess insulin may be produced. On the other hand, where glucose levels of a patient are decreased and rate(s) of change of glucose levels in the patient's body are low, there may be less insulin secretion. Low insulin may lead to limited access of glucose by the cells; thus, extracellular glucose levels may increase.

Insulin is partially removed from circulation by the kidneys. Inhibitors (e.g., SGLT2) may block absorption of glucose at the kidneys. In certain embodiments, the glucose data may be collected by a CGM and may be monitored by the health management system 100 for glycemic variability. Glycemic variability may generally include the standard deviation of glucose levels over a period of time, in addition to time in range (TIR) data. TIR refers to the one or more time periods in which glucose levels of a patient are within a certain desired range (e.g., healthy range).

In certain embodiments, the one or more algorithms and/or models described herein, e.g., for generating a treatment recommendation or for predicting the onset of euDKA, may be configured to use input from one or more sensors measuring one or more of the multiple analytes described above. Parameters and/or thresholds of such algorithms and/or models may be altered based, at least in part, on a number of analytes being measured for input to reflect the knowledge attained from each of the other analytes being measured.

At block 404, the method 400 may optionally include monitoring of non-analyte sensor data during the one or more time periods, using one or more non-analyte sensors or devices (e.g., such as the non-analyte sensors 206 and/or the medical device 208 of FIG. 2).

As mentioned previously, the non-analyte sensors 206 and devices may include one or more of, but are not limited to, an insulin pump, a haptic sensor, an electrocardiogram (ECG) sensor or heart rate monitor, a blood pressure sensor, a sweat sensor, a respiratory sensor, a thermometer, a pulse oximeter, an impedance sensor, sensors or devices provided by display device 107 (e.g., accelerometer, camera, global positioning system (GPS), heart rate monitor, etc.) or other user accessories (e.g., a smart watch), or any other sensors or devices that provide relevant information about the user. One or more of these non-analyte sensors 206 and devices may provide data to the decision support engine 114 described herein. In some aspects, a user, e.g., a patient, may manually input the data for processing by the decision support engine 114.

For example, an accelerometer may be used to determine a physical activity state. Accelerometer data may be analyzed and associated with a change in analyte data (e.g., a sudden downward trend in glucose values) which may be separated from a calculated effect of a medication or other therapy. In another example, a heart rate monitor may be used to provide heart rate data indicative of a physical activity state to further extricate an effect of physical activity from an effect of a medication or other treatment. In another example, a sweat sensor may provide data indicative of a physical activity state to allow for separation of a physical activity factor from an efficacy of a treatment.

Certain metrics, such as one or more of the metrics 130 illustrated in FIG. 3, may be calculated using measured data from each of these additional sensors. Further, as illustrated in FIG. 3, one or more of the metrics 130 calculated from the non-analyte sensor or device data may include body temperature, heart rate (including heart rate variability), respiratory rate, etc. In certain embodiments, described in more detail below, the one or more of the metrics 130 calculated from non-analyte sensor or device data may be used to further inform the treatment recommendation and/or euDKA prediction.

In certain embodiments, one or more non-analyte sensors and/or devices that may be worn by a patient may include a blood pressure sensor. Blood pressure measurements collected from a blood pressure sensor may be used to provide additional insight into health of the patient.

In certain embodiments, one or more non-analyte sensors and/or devices that may be worn by a patient may include an ECG sensor and/or a heart rate monitor. As is known in the art, an ECG device is a device that measures the electric activity of the heartbeat. In certain embodiments, heart rate measurements, as well as heart rate variability information, collected from an ECG sensor and/or a heart rate monitor may be used in combination with ketone and glucose data to better inform the treatment recommendation and/or euDKA prediction.

At block 406, the method 400 continues by processing the analyte data from the one or more time periods, and in certain embodiments, the other non-analyte sensor data, to determine analyte metrics, such as the glucose and/or ketone related metrics 130 discussed in relation to FIG. 3. Block 406, in certain embodiments, may be performed by the decision support engine 114.

In certain embodiments, machine-learning models, described herein, used to provide treatment recommendations and/or euDKA-related predictions may include one or more features not only related to analyte levels of the patient, but also analyte level trends, analyte level rates of change, and similar metrics 130 of the patient. For example, an example machine-learning model may include weights applied to features associated with the one or more trends, rates of change, and/or similar metrics 130 associated with, e.g., ketone levels, glucose levels. Thus, in certain embodiments, prior to use of the machine-learning model, at least some of the metrics 130 relating to ketone and/or glucose levels may be calculated for input into the model.

In some examples, a machine-learning model may implement a fusion of analyte levels (e.g., ketone alone, glucose with ketone, glucose with ketone and with lactate). Non-analyte data (e.g., heart rate, blood pressure, temperature) may also be used in conjunction with analyte levels. In some embodiments, weighting may be applied to features associated with the one or more trends, rates of change, and/or similar metrics for data obtained from analyte sensors (i.e., ketones alone, ketones and glucose) and, in some examples, non-analyte sensors. In some embodiments, using such features will allow for a machine-learning model to be trained to distinguish between a diabetic ketoacidosis and a nutritional ketoacidosis.

Further, in certain embodiments, rule-based models, described herein, used to provide treatment recommendations and/or euDKA predictions, may include one or more rules not only related to analyte levels of the patient, but also analyte metrics (e.g., such as analyte level trends, analyte level rates of change, and other analyte metrics 130) of the patient. For example, a reference library, used to define one or more rules for the rule-based models, may maintain ranges of, e.g., ketone levels, glucose levels, ketone level rates of change, glucose level rates of change, and/or other ketone and glucose metrics, which may be mapped to different treatment recommendations and/or different euDKA onset predictions. Thus, prior to use of the rule-based model, at least some of the metrics 130 relating to ketone and/or glucose levels may be calculated for input into the model. Additional features, such as ketone and/or glucose level rates of change, added to the model may, in some cases, allow for a more accurate treatment recommendation and/or euDKA onset prediction for the patient.

In some examples, a rule-based model may be implemented to differentiate between diabetic ketoacidosis (DKA) and nutritional ketosis (NK). For example, the rule-based model may take into account analyte rates of change. For example, in euDKA, a measure of ketone bodies may rise rapidly (i.e., hours) whereas it takes longer to achieve nutritional ketosis (days). Therefore, by taking into account the rate of change of ketone bodies, a rule-base model is able to differentiate between diabetic ketoacidosis (DKA) and nutritional ketosis (NK). A rule-based model may also take into account absolute levels of ketone bodies. For example, in euDKA ketone levels may be high (i.e. >1 mM-3 mM increased risk and >3 mM very high risk) whereas nutritional ketosis maybe be bounded within about 0.5<NK<3.0 mM. Therefore, by taking into account absolute levels of ketone bodies, a rule-base model is able to differentiate between diabetic ketoacidosis (DKA) and nutritional ketosis (NK).

In another example, both of DKA and nutritional ketosis can be risk-factor-weighted to determine a likelihood of the presence of DKA or nutritional ketosis based on medication(s) taken. In other words, it is likely that ‘classic’ DKA can arise for an insulin-dependent patient with diabetes, euDKA can arise for an insulin-dependent (or non-insulin-dependent) patient with diabetes undergoing SGLT-2 therapy, and nutritional ketosis is more likely for patients on low-carb diets or patients without diabetes. In short, rules to distinguish between DKA, euDKA, and nutritional ketosis may be implemented based on rate of change in analytes, absolute values of analytes, and/or weighted risk factors.

At optional block 408, the method 400 continues by generating a euDKA prediction, which may include: (1) a likelihood or risk that the patient is experiencing (or will experience) euDKA and/or (2) a presence and/or severity of euDKA experienced by the patient using: at least (a) the analyte metrics of the patient (e.g., determined at block 406), including ketone levels, ketone rate of change, and other relevant ketone metrics (as described in relation to FIG. 3), as well as glucose levels, glucose rate of change, and other relevant glucose metrics (as described in relation to FIG. 3); (b) the patient treatment data, and (c) a trained AI/ML model or a rule-based model.

As an example, a user that has been prescribed a certain dosage and frequency of SGLT2 inhibitors may be directed by their physician to use the health management system 100 described above in order to determine whether the prescribed dosage/frequency of SGLT2 inhibitors is safe and well-tolerated by predicting a likelihood of euDKA. In such an example, the user utilizes the analyte sensor system 104, that continuously generates at least glucose and ketone values, as well as software application 106 that receives the glucose and ketone measurements to be used as input into a fusion model for predicting the likelihood of euDKA occurring within a defined time period (e.g., 1 hour, 1 day, 1 week, 1 month, etc.).

In particular, in that example, the prescribed dosage and frequency of SGLT2 inhibitors as well as the user's own glucose and ketone measurements, and/or other user information are used as input to determine a likelihood of euDKA. As an example, after a week of taking the SGLT2 inhibitors, the fusion model may indicate a 90% likelihood of euDKA occurring within, for example, a week of when the prediction is made. In such an example, the likelihood is displayed to the user and/or transmitted to the physician/prescriber, so that the dosage and/or frequency of the inhibitors can be adjusted to lower the likelihood of euDKA. Examples of other user information include other analyte data (analyte data besides glucose and ketone measurements), non-analyte data (e.g., blood pressure, temperature, oxygen saturation, heart rate, accelerometer, etc.), demographic information (e.g., age, gender, ethnicity, etc.), anthropometric information (e.g., height, weight, BMI), clinical chemistry information (e.g., fasting blood glucose level, HbAlc level), compliance with SGLT2 therapy, disease information, diet/meal information, exercise/activity data, renal state information, hydration, body mass index (BMI), etc.

The fusion model described above may be a fusion rule-based model configured to provide real-time decision support for onset of euDKA. A fusion rule-based model herein refers to a rule-based models that takes into account both the patient's ketone and glucose metrics for making determinations (e.g., (1) a likelihood that the patient is experiencing (or will experience) euDKA; (2) a risk of euDKA; or (3) a presence and/or severity of euDKA).

As mentioned previously, rule-based models involve using a set of rules for mapping inputs to outputs. In particular, decision support engine 114 may apply rule-statements (e.g., if, then statements, do-while statements, catch statements, switch statements) to take, as input, the patient's metrics (e.g., analyte metrics or other metrics described in relation to FIG. 3) and treatment data and determine a likelihood of a patient experiencing euDKA within a certain defined period of time, perform a euDKA risk stratification for a patient, and/or identify risks associated with euDKA for the patient.

Rules implemented by the fusion rule-base model may be defined and maintained by the decision support engine 114 in a reference library. For example, the reference library may maintain ranges of metrics, such as analyte metrics (e.g., levels and/or rates of change of glucose and/or ketone) as well as various dosages/frequencies of medications (e.g., inhibitors), which together may be mapped to different likelihoods of the onset of euDKA. In certain embodiments, such rules may be determined based on empirical research as well as analyzing historical patient records from the historical records database 112. For example, an example of a rule may include: if the patient's glucose levels are A, ketone levels are B, prescribed dosage of SGLT2 is C, prescribed frequency of taking SGLT2 is D, then there is a 90% likelihood of the patient experiencing euDKA within a certain defined time period (e.g., 2 mins, 2 hours, 2 days, 2 weeks, 2 months, etc.). The rules may be more granular and complex such that additional analyte and/or non-analyte metrics and other patient information, such as any of the information stored in the patient profile 118, may be used as input for making the determinations described above.

In certain embodiments, the fusion model may be an AI model, such as a machine-learning model (e.g., supervised), used to provide a likelihood of euDKA. In certain embodiments, the decision support engine 114 may deploy one or more fusion machine learning models for predicting euDKA in a patient. Examples of fusion models may include a Bayesian model, regression model, classification model, support vector machine, decision tree, Monte Carlo model, neural network, artificial neural network, convolutional neural network, recurrent neural network, clustering, principal component analysis, discriminant analysis, maximum likelihood estimator, long short-term memory, etc.

In particular, the decision support engine 114 may obtain information from a user profile 118 associated with a patient, stored in the user database 110, featurize information for the patient stored in the user profile 118 into one or more features, and use these features as input into such models. Alternatively, information provided by the user profile 118 may be featurized by another entity and the features may then be provided to the decision support engine 114 to be used as input into the ML models. In certain embodiments, features associated with the patient may be used as input into one or more of the models. Details associated with how one or more machine-learning models can be trained are further discussed in relation to FIG. 5.

For example, a fusion AI model may take, as input, the patient's metrics (e.g., analyte metrics or other metrics described in relation to FIG. 3) and treatment data (e.g., dosage/frequency information) and output a likelihood of a patient experiencing euDKA within a certain defined period of time, a euDKA risk stratification for a patient, or other determinations.

The output(s) of the fusion model(s) (e.g., the rule-based or AI model) may then be used to adjust the patient's treatment. For example, if, based on the patient's metrics and treatment data, the fusion model predicts a 95% likelihood of the patient experiencing euDKA, then the patient's physician may adjust the dosage/frequency of SGLT2. In certain embodiments, the output(s) of the fusion model(s) may be used to provide a health care provider (HCP) intervention recommendation to the patient, which may include a recommendation for the patient to seek medical attention. For example, in certain embodiments, if, based on the patient's metrics and treatment data, the fusion model predicts a 95% likelihood of the patient experiencing euDKA in a defined period of time (e.g., 2 hours), the HCP intervention recommendation may indicate to a patient, or another individual with an interest in the patient's well-being, that the patient needs to immediately go to the emergency room and/or contact their health care provider.

In certain other embodiments, the HCP intervention recommendation may automatically alert the health care provider of the patient as to the condition of the patient for intervention by the physician. In certain other embodiments, the HCP intervention recommendation may alert medical personnel to send aid to the patient, e.g., trigger ambulance services or paramedic services to provide urgent pre-hospital treatment and stabilization to the patient and/or transport of the patient to definitive care. In certain embodiments, decision support engine 114 may make an HCP intervention recommendation based on a patient's ability to seek medical help and/or the accessibility of the patient to medical help.

In some cases, the method 400 continues at block 410 by the decision support engine 114 generating one or more treatment recommendations, based, at least in part, on the patient treatment data collected at block 402 and the analyte metrics generated at blocks 404/406. The treatment recommendations may include recommendation for the optimizing of medication type, dosage, and/or frequency in order to reduce the likelihood of euDKA. In certain embodiments, treatment recommendations may include a recommendation for the patient to stop taking a previously prescribed medication, and in some cases, recommend an alternative medication for consumption by the patient. In certain embodiments, medication recommendations may include a recommendation for the patient to take a lower or higher dosage of a previously prescribed medication. In certain embodiments, treatment recommendations may include a recommendation for the titration of a dosage or timing of a dosage of medication previously prescribed to the patient to determine an ideal dosage for the patient (e.g., while monitoring health of the user). In certain embodiments, recommendations regarding medications may be generated to reduce the risk of euDKA.

To illustrate this with an example, a user that has been prescribed a certain dosage and frequency of SGLT2 inhibitors may be directed by their physician to use the health management system 100 in order to predict an optimal dosage/frequency of the inhibitors. In such an example, the user utilizes the analyte sensor system 104, that continuously generates at least glucose and ketone values, as well as software application 106 that receives the glucose and ketone measurements to be used an input into a fusion model for predicting the optimal dosage/frequency of the inhibitors. In particular, in that example, the user's own glucose and ketone measurements, and/or other user information, are used as input to predict a dosage/frequency for the user that minimizes the risk of euDKA while helping the user achieve euglycemia or otherwise maximize time-in-range.

The treatment recommendations may be provided by a fusion model, such as a rule-based model or an AI/ML model. Using a fusion rule-based model, the decision support engine 114 may apply rule-statements (e.g., if, then statements, do-while statements, catch statements, switch statements) to take, as input, the patient's metrics (e.g., analyte metrics or other metrics described in relation to FIG. 3) and determine an optimized dosage/frequency of SGLT2 consumption to minimize the risk of euDKA. For example, an example of a rule may include: if the patient's glucose levels are A, ketone levels are B, then an optimized dosage and/or frequency of SGLT2 consumption is C. The rules may be more granular and complex such that additional analyte and/or non-analyte metrics and other patient information, such as any of the information stored in the patient profile 118, may be used as input for making the determinations described above.

In certain embodiments, the treatment recommendation may be provided by an AI model, such as a machine-learning model. In certain embodiments, the decision support engine 114 may deploy one or more fusion machine learning models for predicting an optimal dosage and/or frequency of SGLT2 consumption.

In particular, the decision support engine 114 may obtain information from a user profile 118 associated with a patient, stored in the user database 110, featurize information for the patient stored in the user profile 118 into one or more features, and use these features as input into such models. Alternatively, information provided by the user profile 118 may be featurized by another entity and the features may then be provided to the decision support engine 114 to be used as input into the ML models. In certain embodiments, features associated with the patient may be used as input into one or more of the models. Details associated with how one or more machine-learning models can be trained are further discussed in relation to FIG. 5.

For example, a fusion AI model may take, as input, the patient's metrics (e.g., analyte metrics or other metrics described in relation to FIG. 3) and output an optimal dosage and/or frequency of SGLT2 consumption, a euDKA risk stratification for a patient, or other determinations. Note that SGLT2 is used as an example and dosage/frequency of any other inhibitors may similarly be optimized.

After generating the one or more recommendations, at block 412, the method 400 continues by transmitting an indication (e.g., an alert, alarm, or other type of notification) to a user regarding the euDKA-related prediction(s) and/or the generated treatment recommendations, examples of which are described above. In certain embodiments, the indication is transmitted to the patient via the application 106, wherein the indication is displayed to the user on the display device 107 such as a smart phone or other computing device. In certain embodiments, the indication is transmitted to a health care provider, in addition or alternatively to the patient.

In certain embodiments, any one or more components or devices of the health management system 100 may comprise a “share/follow” function to alarm, alert, provide recommendations to, and share historical and/or projected data with healthcare professionals, clinicians, and/or other caregivers of a patient. For example, such “share/follow” function may be included on the one or more continuous analyte sensors 202 of the continuous analyte monitoring system 104 and/or the application 106 as executed on the display device 107. In certain embodiments, such decision support alarms, alerts, and/or recommendations may be tailored to the healthcare professionals, clinicians, and/or caregivers of the patient, rather than the patient. In certain embodiments, such support alarms, alerts, and/or recommendations may be automatically provided to the healthcare professionals, clinicians, and/or other caregivers of the patient. In certain embodiments, a patient may request the health management system 100 to provide such support alarms, alerts, and/or recommendations to the healthcare professionals, clinicians, and/or other caregivers of the patient, via patient interaction with, e.g., an interface of a display device 107 associated with the patient or a continuous analyte sensor 202. The support alarms, alerts, and/or recommendations may generally be provided to the healthcare professionals, clinicians, and/or other caregivers of the patient through wired/wireless communication, and/or other means of communicating data.

In certain embodiments, machine learning models deployed by the decision support engine 114 include one or more models trained by the training server system 140, as illustrated in FIG. 1. FIG. 5 describes in further detail techniques for training the machine learning model(s) deployed by the decision support engine 114 for generating treatment recommendations and/or euDKA predictions, according to certain embodiments of the present disclosure.

In certain embodiments, the method 500 is used to train models to generate, as output, treatment recommendations and/or predictions associated with euDKA for patients. The method 500 begins, at block 502, by a training server system, such as the training server system 140 illustrated in FIG. 1, retrieving data from a historical records database or an electronic medical record/electronic health record, such as the historical records database 112 illustrated in FIG. 1. As mentioned herein, the historical records database 112 may provide a repository of up-to-date information and historical information for users of a continuous analyte monitoring system and connected mobile health application, such as users of the continuous analyte monitoring system 104 and the application 106 illustrated in FIG. 1, as well as data for one or more patients who are not, or were not previously, users of the continuous analyte monitoring system 104 and/or the application 106. In certain embodiments, the historical records database 112 may include one or more data sets of historical patients with no instances of inhibitor use and/or instances of euDKA.

Retrieval of data from the historical records database 112 by the training server system 140, at block 502, may include the retrieval of all, or any subset of, information maintained by the historical records database 112. For example, where the historical records database 112 stores information for 100,000 patients (e.g., non-users and users of the continuous analyte monitoring system 104 and the application 106), data retrieved by the training server system 140 to train one or more machine learning models may include information for all 100,000 patients or only a subset of the data for those patients, e.g., data associated with only 50,000 patients or only data from the last ten years.

As an illustrative example, integrating with on premises or cloud based medical record databases through Fast Healthcare Interoperability Resources (FHIR), web application programming interfaces (APIs), Health Level 7 (HL7), and or other computer interface language may enable aggregation of healthcare historical records for baseline assessment in addition to the aggregation of de-identifiable patient data from a cloud based repository.

As an illustrative example, at block 502, the training server system 140 may retrieve information for 100,000 patients under treatment with inhibitors (e.g., SGLT2) stored in the historical records database 112 to train a model to determine a treatment recommendation for optimizing use of the inhibitor in a user. Each of the 100,000 patients may have a corresponding data record (e.g., based on their corresponding user profile), stored in the historical records database 112. Each user profile 118 may include information, such as information discussed with respect to FIG. 3.

The training server system 140 then uses information in each of the records to train an artificial intelligence or ML model (for simplicity referred to as “ML model” herein). Examples of types of information included in a patient's user profile were provided above. The information in each of these records may be featurized (e.g., manually or by the training server system 140), resulting in features that can be used as input features for training the ML model. For example, a patient record may include or be used to generate features related to an age of a patient, a gender of the patient, a BMI of a patient, an occupation of the patient, analyte metrics (e.g., analyte levels for the patient over time, analyte level rates of change and/or trends for the patient over time) physiological parameters for the patient over time, treatment data, disease information, etc. Features used to train the machine learning model(s) may vary in different embodiments.

In certain embodiments, each historical patient record retrieved from the historical records database 112 is further associated with a label indicating the dosage and/or frequency of inhibitor consumption, information relating to euDKA (e.g., whether the user experienced euDKA), and/or similar metrics. What the record is labeled with would depend on what the model is being trained to predict.

At block 504, the method 500 continues by the training server system 140 training one or more machine learning models based on the features and labels associated with the historical patient records. In some embodiments, the training server does so by providing the features as input into a model. This model may be a new model initialized with random weights and parameters, or may be partially or fully pre-trained (e.g., based on prior training rounds). Based on the input features, the model-in-training generates some output. In certain embodiments, the output may recommend a treatment or change in treatment of the user or provide a prediction of euDKA.

In certain embodiments, the training server system 140 compares this generated output with the actual label associated with the corresponding historical patient record to compute a loss based on the difference between the actual result and the generated result. This loss is then used to refine one or more internal weights and parameters of the model (e.g., via backpropagation) such that the model learns to more accurately predict optimized treatment recommendations and/or the onset of euDKA.

One of a variety of machine learning algorithms may be used for training the model(s) described above. For example, one of a supervised learning algorithm, a neural network algorithm, a deep neural network algorithm, a deep learning algorithm, etc. may be used.

At block 506, the training server system 140 deploys the trained model(s) to make treatment recommendations and/or predictions associated with euDKA. In some embodiments, this includes transmitting some indication of the trained model(s) (e.g., a weights vector) that can be used to instantiate the model(s) on another device. For example, the training server system 140 may transmit the weights of the trained model(s) to the decision support engine 114. The model(s) can then be used to assess, in real-time, the treatment of the user, predict onset of euDKA, provide treatment recommendations, etc. In certain embodiments, the training server system 140 may continue to train the model(s) in an “online” manner by using input features and labels associated with new patient records.

Further, similar methods for training as that illustrated in FIG. 5 using historical patient records may also be used to train models using patient-specific records to create more personalized models for making treatment recommendations and/or predictions associated with euDKA. For example, a model trained using historical patient records that is deployed for a particular user, may be further re-trained after deployment. For example, the model may be re-trained after the model is deployed for a specific patient to create a more personalized model for the patient. The more personalized model may be able to make more accurate treatment recommendations and/or predict euDKA for the patient based on the patient's own data (as opposed to only historical patient record data), including the patient's own analyte (e.g., ketone, glucose) metrics. In some embodiments, training of the model may include individualizing the model based on population health metrics. For example, tuning of the model with segments of the training dataset associated with patients who share one or more aspects with the patient may allow for more refined and individualized treatment recommendations.

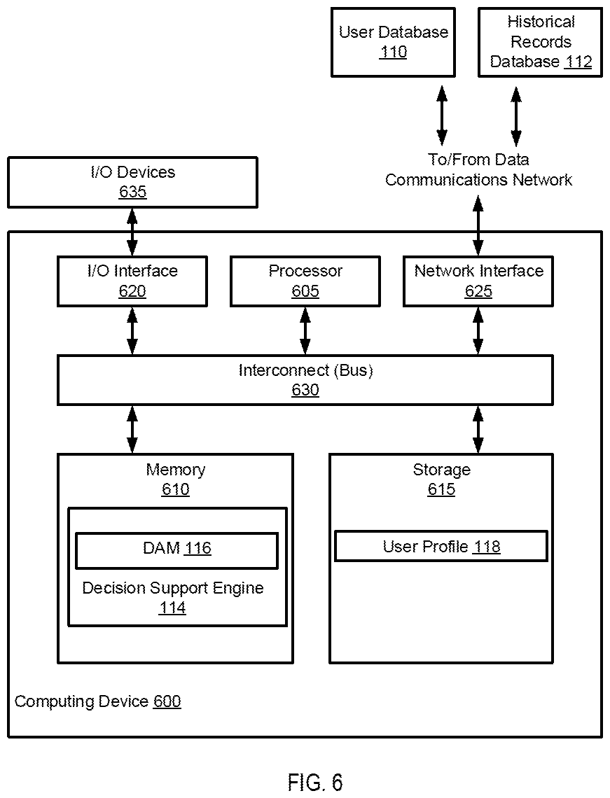

FIG. 6 is a block diagram depicting a computing device 600 configured for providing treatment recommendations and/or predicting onset of euDKA, according to certain embodiments disclosed herein. Although depicted as a single physical device, in embodiments, the computing device 600 may be implemented using virtual device(s), and/or across a number of devices, such as in a cloud environment. As illustrated, the computing device 600 includes a processor 605, a memory 610, a storage 615, a network interface 625, and one or more I/O interfaces 620. In the illustrated embodiment, the processor 605 retrieves and executes programming instructions stored in the memory 610, as well as stores and retrieves application data residing in the storage 615. The processor 605 is generally representative of a single CPU and/or GPU, multiple CPUs and/or GPUs, a single CPU and/or GPU having multiple processing cores, and the like.

The memory 610 is generally included to be representative of a random access memory (RAM). The storage 615 may be any combination of disk drives, flash-based storage devices, and the like, and may include fixed and/or removable storage devices, such as fixed disk drives, memory modules, removable memory cards, embedded memory, on-die memory, caches, optical storage, network attached storage (NAS), or storage area networks (SAN).

In some embodiments, the I/O devices 635 (such as keyboards, monitors, etc.) can be connected via the I/O interface(s) 620. Further, via the network interface 625, the computing device 600 can be communicatively coupled with one or more other devices and components, such as the user database 110 and/or the historical records database 112. In certain embodiments, the computing device 600 is communicatively coupled with other devices via a network, which may include the Internet, local network(s), and the like. The network may include wired connections, wireless connections, or a combination of wired and wireless connections. As illustrated, the processor 605, memory 610, storage 615, network interface(s) 625, and the I/O interface(s) 620 are communicatively coupled by one or more interconnects 630. In certain embodiments, the computing device 600 is representative of the display device 107 associated with the user. In certain embodiments, as discussed above, the display device 107 can include the user's laptop, computer, smartphone, and the like. In another embodiment, the computing device 600 is a server executing in a cloud environment.

In the illustrated embodiment, the storage 615 includes the user profile 118. The memory 610 includes the decision support engine 16, which itself includes the DAM 116. The decision support engine 114 is executed by the computing device 600 to perform operations in the method 400 of FIG. 4 and operations of the method 500 in FIG. 5 for providing decision support in the form of treatment recommendations and/or euDKA onset predictions.

Embodiments described herein may allow for an increase in the efficacy and safety of pharmacologic agents (e.g., inhibitors, agonists, hormones such as insulin, etc.) by providing recommendations for dosage and frequency of the pharmacologic agent for improved therapeutic effect and a reduction in negative effects (e.g., euDKA). Recommendations may be provided automatically or via healthcare provider review and input.

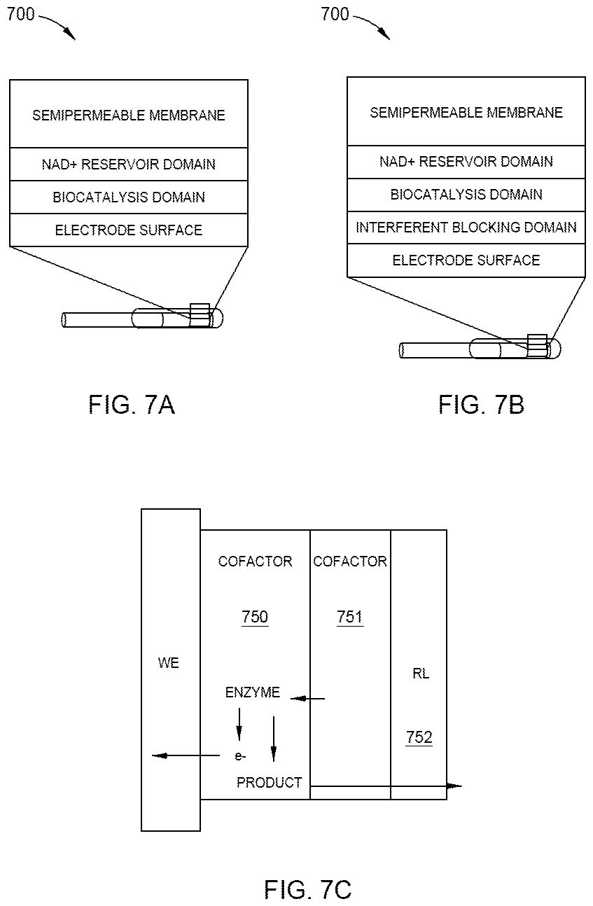

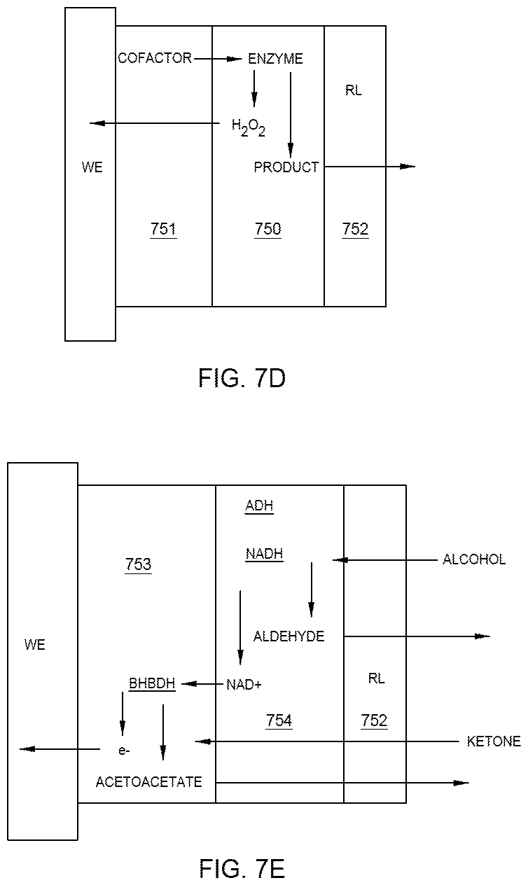

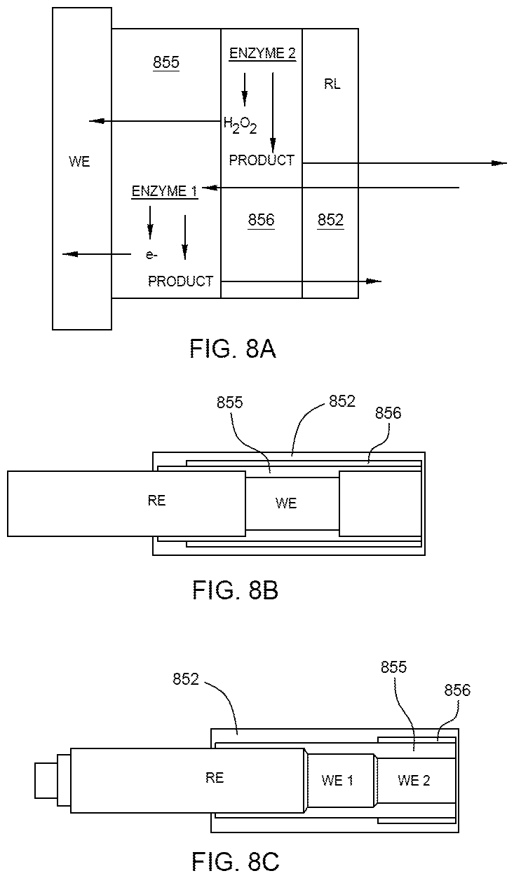

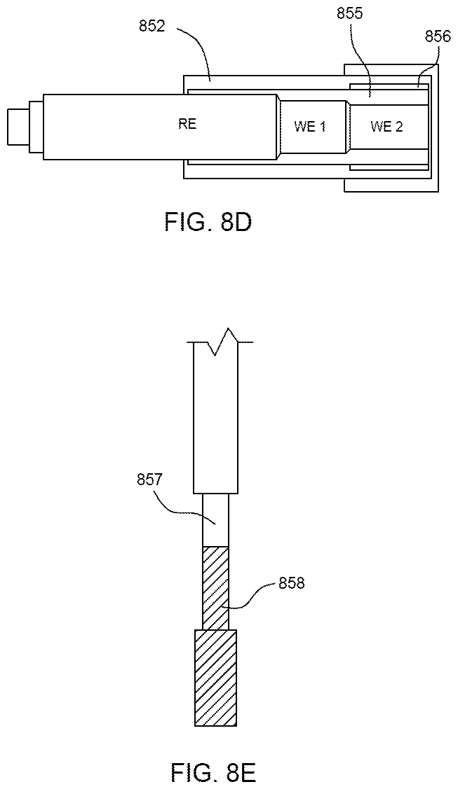

As described above, continuous analyte monitoring system 104, described in relation to FIG. 1, may be a multi-analyte sensor system including a multi-analyte sensor. FIGS. 7-8 describe example multi-analyte sensors used to measure multiple analytes.

The following description and figures illustrate examples of the present disclosure in detail. Those of skill in the art will recognize that there are numerous variations and modifications of this disclosure that are encompassed by its scope. Accordingly, the examples herein should not be deemed to limit the scope of the present disclosure. In order to facilitate an understanding of the examples disclosed herein, a number of terms are defined below.

The term “about” as used herein is a broad term, and is to be given its ordinary and customary meaning to a person of ordinary skill in the art (and is not to be limited to a special or customized meaning), and refers without limitation to allowing for a degree of variability in a value or range, for example, within 10%, within 5%, or within 1% of a stated value or of a stated limit of a range, and includes the exact stated value or range.

The term “adhere” and “attach” as used herein are broad terms, and are to be given their ordinary and customary meaning to a person of ordinary skill in the art (and are not be limited to a special or customized meaning), and refer without limitation to hold, bind, or stick, for example, by gluing, bonding, grasping, interpenetrating, or fusing.

The term “analyte” as used herein is a broad term, and is to be given its ordinary and customary meaning to a person of ordinary skill in the art (and is not to be limited to a special or customized meaning), and refers without limitation to a substance or chemical constituent in a biological fluid (e.g., blood, interstitial fluid, cerebral spinal fluid, lymph fluid, urine, sweat, saliva, etc.) that can be analyzed. Analytes can include naturally occurring substances, artificial substances, metabolites, and/or reaction products. In some examples, the analyte measured by the sensing regions, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); bilirubin, biotinidase; biopterin; c-reactive protein; carnitine; carnosinase; CD4; ceruloplasmin; chenodeoxycholic acid; chloroquine; cholesterol; cholinesterase; conjugated 1-βhydroxy-cholic acid; cortisol; creatine; creatine kinase; creatine kinase MM isoenzyme; creatinine; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycerol; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; beta-hydroxybutyrate; ketones; lactate; lead; lipoproteins ((a), B/A-1, β); lysozyme; mefloquine; netilmicin; oxygen; phenobarbitone; phenytoin; phytanic/pristanic acid; potassium, sodium, and/or other blood electrolytes; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, Leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uric acid; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain examples. The analyte can be naturally present in the biological fluid, or endogenous, for example, a metabolic product, a hormone, an antigen, an antibody, and the like. Alternately, the analyte can be introduced into the body, or exogenous, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, RITALIN®, CYLERT®, PRELUDIN®, DIDREX®, PRESTATE®, VORANIL®, SANDREX®, PLEGINE®); depressants (barbiturates, methaqualone, tranquilizers such as VALIUM@, LIBRIUM®, MILTOWN®, SERAX®, EQUANIL®, TRANXENE@); hallucinogens (phencyclidine, lysergic acid, mescaline, peyote, psilocybin); narcotics (heroin, codeine, morphine, opium, meperidine, PERCOCET®, PERCODAN®, TUSSIONEX®, fentanyl, DARVON®, TALWIN®, LOMOTIL®); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC®), homovanillic acid (HVA), 5-hydroxytryptamine (5HT), 5-hydroxyindoleacetic acid (FHIAA), and histamine.

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases may refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing (detecting) element.

The term “amphiphilic” as used herein is a broad term and is to be given its ordinary and customary meaning to a person of ordinary skill in the art (and are not to be limited to a special or customized meaning), and refer without limitation to a chemical compound or polymer possessing both hydrophilic and hydrophobic segments or properties.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In one example, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In one example, such biosensors and/or sensors are capable of providing specific quantitative, semi-quantitative, qualitative, semi qualitative analytical signals using a biological recognition element combined with a transducing (detecting) element.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms can provide specific quantitative, semi-quantitative, qualitative, semi qualitative analytical signals using a biological recognition element combined with a transducing (detecting) element.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The phrase “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “adhere” and “attach” as used herein are broad terms, and are to be given their ordinary and customary meaning to a person of ordinary skill in the art (and are not to be limited to a special or customized meaning), and refer without limitation to hold, bind, or stick, for example, by gluing, bonding, grasping, interpenetrating, or fusing.

The phrase “barrier cell layer” as used herein is a broad phrase, and is to be given its ordinary and customary meaning to a person of ordinary skill in the art (and is not to be limited to a special or customized meaning), and refers without limitation to a part of a foreign body response that forms a cohesive monolayer of cells (for example, macrophages and foreign body giant cells) that substantially block the transport of molecules and other substances to the implantable device.

The term “bioactive agent” as used herein is a broad term, and is to be given its ordinary and customary meaning to a person of ordinary skill in the art (and is not to be limited to a special or customized meaning), and refers without limitation to any substance that has an effect on or elicits a response from living tissue.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The term “biostable” as used herein is a broad term, and is to be given its ordinary and customary meaning to a person of ordinary skill in the art (and is not to be limited to a special or customized meaning), and refers without limitation to materials that are relatively resistant to degradation by processes that are encountered in vivo.

The phrase “cell processes” as used herein is a broad phrase, and is to be given its ordinary and customary meaning to a person of ordinary skill in the art (and is not to be limited to a special or customized meaning), and refers without limitation to pseudopodia of a cell.

The phrase “cellular attachment” as used herein is a broad phrase, and is to be given its ordinary and customary meaning to a person of ordinary skill in the art (and is not to be limited to a special or customized meaning), and refers without limitation to adhesion of cells and/or cell processes to a material at the molecular level, and/or attachment of cells and/or cell processes to microporous material surfaces or macroporous material surfaces. One example of a material used in the prior art that encourages cellular attachment to its porous surfaces is the BIOPORE™ cell culture support marketed by Millipore (Bedford, Mass.), and as described in Brauker et al., U.S. Pat. No. 5,741,330.

The term “cofactor” as used herein is a broad term, and is to be given its ordinary and customary meaning to a person of ordinary skill in the art (and is not to be limited to a special or customized meaning), and refers without limitation to one or more substances whose presence contributes to or is required for analyte-related activity of an enzyme. Analyte-related activity can include, but is not limited to, any one of or a combination of binding, electron transfer, and chemical transformation. Cofactors are inclusive of coenzymes, non-protein chemical compounds, metal ions and/or metal organic complexes. Coenzymes are inclusive of prosthetic groups and co-substrates.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrases “continuous analyte sensing” and “continuous multi-analyte sensing” as used herein are broad phrases, and are to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7, 10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks.

The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e. “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components. covalently, electrostatically, ionically associated with, or physically entrapped, or absorbed

The phrase “defined edges” as used herein is a broad phrase, and is to be given its ordinary and customary meaning to a person of ordinary skill in the art (and is not to be limited to a special or customized meaning), and refers without limitation to abrupt, distinct edges or borders among layers, domains, coatings, or portions. “Defined edges” are in contrast to a gradual transition between layers, domains, coatings, or portions.

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of a membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “drift” as used herein is a broad term, and is to be given its ordinary and customary meaning to a person of ordinary skill in the art (and is not to be limited to a special or customized meaning), and refers without limitation to a progressive increase or decrease in signal over time that is unrelated to changes in host systemic analyte concentrations (e.g., host postprandial glucose concentrations). While not wishing to be bound by theory, it is believed that drift may be the result of a local decrease in glucose transport to the sensor, for example, due to formation of a foreign body capsule (FBC), or due to an insufficient amount of interstitial fluid surrounding the sensor, which results in reduced oxygen and/or glucose transport to the sensor. In one example, an increase in local interstitial fluid may slow or reduce drift and thus improve sensor performance. Drift may also be the result of sensor electronics, or algorithmic models used to compensate for noise or other anomalies that can occur with electrical signals in, for example, the picoAmp range, the femtoAmp range, the nanoAmp range, the microAmp range, the milliAmp range, the Amp range, etc.

The phrases “drug releasing membrane” and “drug releasing layer” as used interchangeably herein are each a broad phrase, and each are to be given its ordinary and customary meaning to a person of ordinary skill in the art (and is not to be limited to a special or customized meaning), and refers without limitation to a permeable or semi-permeable membrane which is permeable to one or more bioactive agents. In one example, the “drug releasing membrane” and “drug releasing layer” is typically of a few microns thickness or more and can be comprised of two or more domains. In one example the drug releasing layer and/or drug releasing membrane are substantially the same as the biointerface layer and/or biointerface membrane. In another example, the drug releasing layer and/or drug releasing membrane are distinct from the biointerface layer and/or biointerface membrane.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In one example this reaction is faradaic and results in charge transfer between the surface and its environment. In one example, hydrogen peroxide produced by an enzyme-catalyzed reaction of an analyte being oxidized on the surface results in a measurable electronic current. For example, in the detection of glucose, glucose oxidase produces hydrogen peroxide (H₂O₂) as a byproduct. The H₂O₂ reacts with the surface of the working electrode to produce two protons (2H⁺), two electrons (2e⁻) and one molecule of oxygen (O₂), which produces the electronic current being detected. In a counter electrode, a reducible species, for example, O₂ is reduced at the electrode surface so as to balance the current generated by the working electrode.

The term “electrolysis” as used herein is a broad term, and is to be given its ordinary and customary meaning to a person of ordinary skill in the art (and is not to be limited to a special or customized meeting), and refers without limitation to electrooxidation or electroreduction (collectively, “redox”) of a compound, either directly or indirectly, by one or more enzymes, cofactors, or mediators.

The phrase “hard segment” as used herein is a broad phrase, and is to be given its ordinary and customary meaning to a person of ordinary skill in the art (and is not to be limited to a special or customized meaning), and refers without limitation to an element of a copolymer, for example, a polyurethane, a polycarbonate polyurethane, or a polyurethane urea copolymer, which imparts resistance properties, e.g., resistance to bending or twisting. The phrase “hard segment” can be further characterized as a crystalline, semi-crystalline, or glassy material with a glass transition temperature determined by dynamic scanning calorimetry (“Tg”) typically above ambient temperature. Exemplary hard segment elements used to prepare a polycarbonate polyurethane, or a polyurethane urea hard segment include norbornane diisocyanate (NBDI), isophorone diisocynate (IPDI), tolylene diisocynate (TDI), 1,3-phenylene diisocyanate (MPDI), trans-1,3-bis(isocynatomethyl) cyclohexane (1,3-H6XDI), bicyclohexylmethane-4,4′-diisocynate(HMDI), 4,4′-Diphenylmethane diisocynate (MDI), trans-1,4-bis(isocynatomethyl) cyclohexane (1,4-H6XDI), 1,4-cyclohexyl diisocynate (CHDI), 1,4-phenylene diisocynate (PPDI), 3,3′-Dimethyl-4,4′-biphenyldiisocyanate (TODI), 1,6-hexamethylene diisocyanate (HDI), or combinations thereof.

The term “host” as used herein is a broad term, and is to be given its ordinary and customary meaning to a person of ordinary skill in the art (and is not to be limited to a special or customized meaning), and refers without limitation to mammals, for example humans.

The terms “indwelling,” “in dwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), or transcutaneously (i.e. penetrating, entering, or passing through intact skin), which may result in a sensor that has an in vivo portion and an ex vivo portion. The term “indwelling” also encompasses an object which is configured to be inserted subcutaneously, intracutaneously, or transcutaneously, whether or not it has been inserted as such.

The phrase “insertable surface area” as used herein is a broad phrase, and is to be given its ordinary and customary meaning to a person of ordinary skill in the art (and is not to be limited to a special or customized meaning), and refers without limitation to a surface area of an insertable portion of an analyte sensor including, but not limited to, the geometric surface area e.g., planar, flat or substantially planar, and/or coaxial utilized substrates in the analyte sensor as described herein.

The phrase “insertable volume” as used herein is a broad phrase, and is to be given its ordinary and customary meaning to a person of ordinary skill in the art (and is not to be limited to a special or customized meaning), and refers without limitation to a volume ahead of and alongside a path of insertion of an insertable portion of an analyte sensor, as described herein, as well as an incision made in the skin to insert the insertable portion of the analyte sensor. The insertable volume also includes up to 5 mm radially or perpendicular to the volume ahead of and alongside the path of insertion.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are compounds which produce a signal that is not analyte-specific due to a reaction on an electrochemically active surface.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

As used herein, the terms “machine-storage medium,” “device-storage medium,” “computer-storage medium” (referred to collectively as “machine-storage medium” mean the same thing and may be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media include non-volatile memory, including by way of example semiconductor memory devices, e.g., erasable programmable read-only memory (EPROM), electrically erasable programmable devices; magnetic disks such as internal hard disks and removable disks; magneto-optical disks; and CD-ROM and DVD-ROM disks. The terms machine-storage media, computer-storage media, and device-storage media specifically exclude carrier waves, modulated data signals, and other such media, at least some of which are covered under the term “signal medium” discussed below.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In one example the mediator accepts electrons from, or transfer electrons to, one or more enzymes or cofactors, and/or exchanges electrons with the sensor system electrodes. In one example, mediators include electroreducible and electrooxidizable ions, complexes or having oxidation-reduction potentials above or below the oxidation-reduction potential of a standard calomel electrode (SCE), for example, about 50, 75, 100, 150, 200, 250, 300, 350, 400, 450, 500, 550, 600, 650, 700, or 750 millivolts above or below the oxidation-reduction potential of the SCE are transition-metal coordinated organic molecules which are capable of reversible oxidation and reduction reactions. In other examples, mediators may be organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In one example, the membrane system comprises an enzyme, which enables an analyte reaction to occur whereby a concentration of the analyte can be measured.

The phrases “machine-readable medium,” ‘computer-readable medium” and ‘device-readable medium” mean the same thing and may be used interchangeably in this disclosure. The phrases are inclusive of both machine-storage media and signal media operably coupled to a sensor, biosensor, analyte sensing device, or analyte monitoring device. Thus, the phrases are inclusive of both storage devices/media and carrier waves/modulated data signals operably coupled to a sensor, biosensor, analyte sensing device, or analyte monitoring device.

The term “micro,” as used herein is a broad term, and is to be given its ordinary and customary meaning to a person of ordinary skill in the art (and is not to be limited to a special or customized meaning), and refers without limitation to a small object or scale of approximately 10⁻⁶ m that is not visible without magnification. The term “micro” is in contrast to the term “macro,” which refers to a large object that may be visible without magnification. Similarly, the term “nano” refers to a small object or scale of approximately 10⁻⁹ m.

The term “noise,” as used herein, is a broad term and is used in its ordinary sense, including, without limitation, a signal detected by the sensor or sensor electronics that is unrelated to analyte concentration and can result in reduced sensor performance. One type of noise has been observed during the few hours (e.g., about 2 to about 24 hours) after sensor insertion. After the first 24 hours, the noise may disappear or diminish, but in some hosts, the noise may last for about three to four days. In some cases, noise can be reduced using predictive modeling, artificial intelligence, and/or algorithmic means. In other cases, noise can be reduced by addressing immune response factors associated with the presence of the implanted sensor, such as using a drug releasing layer with at least one bioactive agent. For example, noise of one or more exemplary biosensors as presently disclosed can be determined and then compared qualitatively or quantitatively. By way of example, by obtaining a raw signal timeseries with a fixed sampling interval (in units of pA), a smoothed version of the raw signal timeseries can be obtained, e.g., by applying a 3rd order lowpass digital Chebyshev Type II filter. Others smoothing algorithms can be used. At each sampling interval, an absolute difference, in units of pA, can be calculated to provide a smoothed timeseries. This smoothed timeseries can be converted into units of mg/dL, (the unit of “noise”), using a glucose sensitivity timeseries, in units of pA/mg/dL, where the glucose sensitivity timeseries is derived by using a mathematical model between the raw signal and reference blood glucose measurements (e.g., obtained from Blood Glucose Meter). Optionally, the timeseries can be aggregated as desired, e.g., by hour or day. Comparison of corresponding timeseries between different exemplary biosensors with the presently disclosed drug releasing layer and one or more bioactive agents provides for qualitative or quantitative determination of improvement of noise.

The term “optional” or “optionally” as used herein is a broad term, and is to be given its ordinary and customary meaning to a person of ordinary skill in the art (and is not to be limited to a special or customized meaning), and, without limitation, means that the subsequently described event or circumstance can or cannot occur, and that the description includes instances where the event or circumstance occurs and instances where it does not.

The term “planar” as used herein is to be interpreted broadly to describe sensor architecture having a substrate including at least a first surface and an opposing second surface, and for example, comprising a plurality of elements arranged on one or more surfaces or edges of the substrate. The plurality of elements can include conductive or insulating layers or elements configured to operate as a circuit. The plurality of elements may or may not be electrically or otherwise coupled. In one example, planar includes one or more edges separating the opposed surfaces.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme domain or layer. If the sensor is deemed to be the point of reference and the enzyme domain is positioned nearer to the sensor than the biointerface layer, then the enzyme domain is more proximal to the sensor than the biointerface layer.

The phrase and term “processor module” and “microprocessor” as used herein are each a broad phrase and term, and are to be given their ordinary and customary meaning to a person of ordinary skill in the art (and are not to be limited to a special or customized meaning), and refer without limitation to a computer system, state machine, processor, or the like designed to perform arithmetic or logic operations using logic circuitry that responds to and processes the basic instructions that drive a computer.

The term “semi-continuous” as used herein is a broad term, and is to be given its ordinary and customary meaning to a person of ordinary skill in the art (and is not to be limited to a special or customized meaning), and refers without limitation to a portion, coating, domain, or layer that includes one or more continuous and non-continuous portions, coatings, domains, or layers. For example, a coating disposed around a sensing region but not about the sensing region is “semi-continuous.”

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms are capable of providing specific quantitative, semi-quantitative, qualitative, semi qualitative analytical signals using a biological recognition element combined with a transducing and/or detecting element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, ketone, lactate, potassium, etc., in the biological sample.

In one example, the sensing region or sensing portion can comprise at least a portion of a conductive substrate or at least a portion of a conductive surface, for example, a wire (coaxial) or conductive trace or a substantially planar substrate including substantially planar trace(s), and a membrane. In one example, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) including mutant or fusion protein thereof, or aptamers having one or more analyte binding regions, each region capable of specifically and reversibly binding to at least one analyte. Alterations of the aptamer or mutations of the PBP can contribute to or alter one or more of the binding constants, long-term stability of the protein, including thermal stability, to bind the protein to a special encapsulation matrix, membrane or polymer, or to attach a detectable reporter group or “label” to indicate a change in the binding region or transduce a signal corresponding to the one or more analytes present in the biological fluid. Specific examples of changes in the binding region include, but are not limited to, hydrophobic/hydrophilic environmental changes, three-dimensional conformational changes, changes in the orientation of amino/nucleic acid side chains in the binding region of proteins, and redox states of the binding region. Such changes to the binding region provide for transduction of a detectable signal corresponding to the one or more analytes present in the biological fluid.

In one example, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoAmps of current for every 1 mg/dL of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The phrases and terms “small diameter sensor,” “small structured sensor,” and “micro-sensor” as used herein are broad phrases and terms, and are to be given their ordinary and customary meaning to a person of ordinary skill in the art (and are not to be limited to a special or customized meaning), and refer without limitation to sensing mechanisms that are less than about 2 mm in at least one dimension. In further examples, the sensing mechanisms are less than about 1 mm in at least one dimension. In some examples, the sensing mechanism (sensor) is less than about 0.95, 0.9, 0.85, 0.8, 0.75, 0.7, 0.65, 0.6, 0.5, 0.4, 0.3, 0.2, or 0.1 mm. In some examples, the maximum dimension of an independently measured length, width, diameter, thickness, or circumference of the sensing mechanism does not exceed about 2 mm. In some examples, the sensing mechanism is a coaxial sensor, wherein the diameter of the sensor is less than about 1 mm, see, for example, U.S. Pat. No. 6,613,379 to Ward et al. and U.S. Pat. No. 7,497,827 to Brister et al., both of which are incorporated herein by reference in their entirety. In some alternate examples, the sensing mechanism includes electrodes deposited on a planar or substantially planar substrate, wherein the thickness of the implantable portion is less than about 1 mm, see, for example U.S. Pat. No. 6,175,752 to Say et al. and U.S. Pat. No. 5,779,665 to Mastrototaro et. al., both of which are incorporated herein by reference in their entirety.

The phrase “soft segment” as used herein is a broad phrase, and is to be given its ordinary and customary meaning to a person of ordinary skill in the art (and is not to be limited to a special or customized meaning), and refers without limitation to an element of a copolymer, for example, a polyurethane, a polycarbonate-polyurethane, or a polyurethane urea copolymer, which imparts flexibility to the chain. The phrase “soft segment” can be further characterized as an amorphous material with a low Tg, e.g., a Tg not typically higher than ambient temperature or normal mammalian body temperature.

The phrase “solid portions” as used herein is a broad term, and is to be given its ordinary and customary meaning to a person of ordinary skill in the art (and is not to be limited to a special or customized meaning), and refers without limitation to portions of a membrane's material having a mechanical structure that demarcates cavities, voids, or other non-solid portions.

The terms “transducing” or “transduction” and their grammatical equivalents as are used herein are broad terms, and are to be given their ordinary and customary meaning to a person of ordinary skill in the art (and is not to be limited to a special or customized meaning), and refer without limitation to optical, electrical, electrochemical, acoustical/mechanical, or colorimetrical technologies and methods. Electrochemical properties include current and/or voltage, inductance, capacitance, impedance, transconductance, and potential. Optical properties include absorbance, fluorescence/phosphorescence, fluorescence/phosphorescence decay rate, wavelength shift, dual wave phase modulation, bio/chemiluminescence, reflectance, light scattering, and refractive index. For example, the sensing region transduces the recognition of analytes into a semi-quantitative or quantitative signal.

As used herein, the phrase “transducing element” as used herein is a broad phrase, and are to be given their ordinary and customary meaning to a person of ordinary skill in the art (and is not to be limited to a special or customized meaning), and refers without limitation to analyte recognition moieties capable of facilitating, directly or indirectly, with detectable signal transduction corresponding to the presence and/or concentration of the recognized analyte. In one example, a transducing element is one or more enzymes, one or more aptamers, one or more ionophores, one or more capture antibodies, one or more proteins, one or more biological cells, one or more oligonucleotides, and/or one or more DNA or RNA moieties. Transcutaneous continuous multi-analyte sensors can be used in vivo over various lengths of time. The continuous multi-analyte sensor systems discussed herein can be transcutaneous devices, in that a portion of the device may be inserted through the host's skin and into the underlying soft tissue while a portion of the device remains on the surface of the host's skin. In one aspect, in order to overcome the problems associated with noise or other sensor function in the short-term, one example employs materials that promote formation of a fluid pocket around the sensor, for example architectures such as a porous biointerface membrane or matrices that create a space between the sensor and the surrounding tissue. In some examples, a sensor is provided with a spacer adapted to provide a fluid pocket between the sensor and the host's tissue. It is believed that this spacer, for example a biointerface material, matrix, structure, and the like as described in more detail elsewhere herein, provides for oxygen and/or glucose transport to the sensor.

### Membrane Systems

Membrane systems disclosed herein are suitable for use with analyte-measuring devices in contact with a biological fluid. For example, the membrane systems can be utilized with analyte-measuring devices, such as devices for monitoring and determining analyte levels in a biological fluid, for example, devices for monitoring glucose levels for individuals having diabetes. In some examples, an analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572, 5,964,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, spraying, electrodepositing, dipping, brush coating, film coating, drop-let coating, and the like). Additional steps may be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (m), or less, to about 1, 2, 3, 4, 5, 6, 7, 8, 9, 10, 11, 12, 13, 14, 15, or 16 m is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like.

Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl)carbodiimide (EDC) or polycarbodiimide crosslinkers) and cured at a moderate temperature of about 50° C. In one example, polycarbodiimide crosslinkers are used.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like.

Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro)chemical reactions involved in sensing, but forms chemical bonds with the (electro)chemically active components of the membrane. In some examples these bonds are covalent. In one example, a tether may be formed in solution prior to one or more interlayers of a membrane being formed, where the tether bonds two (electro)chemically active components directly to one another or alternately, the tether(s) bond (electro)chemically active component(s) to polymeric backbone structures. In another example, (electro)chemically active components are comixed along with crosslinker(s) with tunable lengths (and optionally polymers) and the tethering reaction occurs as in situ crosslinking. Tethering may be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as spraying, dipping, casting, electrospinning, vapor deposition, spin coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. In one example, polycarbodiimide crosslinkers are used. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEG-DE), or dicumyl peroxide (DCP). In one example, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spraying, self-assembling monolayers (SAMs), painting, dip-coating, vapor depositing, molding, 3-D printing, slot die coating, pico jet printing, piezo inkjet printing, lithographic techniques (e.g., photolithograph), micro- and nano-pipetting printing techniques, silk-screen printing, etc.). The mixture can then be cured under high temperature (e.g., from about 30° C. to about 150° C.). Other suitable curing methods can include ultraviolet, e-beam, or gamma radiation, for example.

In some circumstances, using continuous multianalyte monitoring systems including sensor(s) configured with bioprotective and/or drug releasing membranes, it is believed that that foreign body response is the dominant event surrounding extended implantation of an implanted device and can be managed or manipulated to support rather than hinder or block analyte transport. In another aspect, in order to extend the lifetime of the sensor, one example employs materials that promote vascularized tissue ingrowth, for example within a porous biointerface membrane. For example, tissue in-growth into a porous biointerface material surrounding a sensor may promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous bioprotective materials that surround the sensor and promote ingrowth of tissue and microvasculature over time.

Accordingly, a sensor as discussed in examples herein may include a biointerface layer. The biointerface layer, like the drug releasing layer, may include, but is not limited to, for example, porous biointerface materials including a solid portion and interconnected cavities, all of which are described in more detail elsewhere herein. The biointerface layer can be employed to improve sensor function in the long term (e.g., after tissue ingrowth).

Accordingly, a sensor as discussed in examples herein may include a drug releasing membrane at least partially functioning as or in combination with a biointerface membrane. The drug releasing membrane may include, for example, materials including a hard-soft segment polymer with hydrophilic and optionally hydrophobic domains, all of which are described in more detail elsewhere herein, can be employed to improve sensor function in the long term (e.g., after tissue ingrowth). In one example, the materials including a hard-soft segment polymer with hydrophilic and optionally hydrophobic domains are configured to release a combination of a derivative form of dexamethasone or dexamethasone acetate with dexamethasone such that one or more different rates of release of the anti-inflammatory is achieved and the useful life of the sensor is extended. Other suitable drug releasing membranes of the present disclosure can be selected from silicone polymers, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), poly vinyl acetate, ethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(l-lysine), poly(L-lactic acid), hydroxyethylmetharcrylate and copolymers and blends thereof, and hydroxyapeptite and copolymers and blends thereof.

### Sensing Mechanism

In general, the analyte sensors of the present disclosure include a sensing mechanism with a small structure (e.g., small structured-, micro- or small diameter sensor), for example, a coaxial or planar sensor, in at least a portion thereof. As used herein a “small structure” refers to an architecture with at least one dimension less than about 1 mm. The small-structured sensing mechanism can be coaxial-based, or substrate-based (flat or substantially planar substrate, that can be single or double-sided, which may or may include one or more sensor elements on any of the sides or surfaces), or any other architecture. In some alternate examples, the term “small structure” can also refer to slightly larger structures, such as those having their smallest dimension being greater than about 1 mm, however, the architecture (e.g., mass or size) is designed to minimize the foreign body response due to size and/or mass.

The present disclosure is inclusive of sensor systems including two or more sensors, each sensor being configured to sense a different analyte. The two or more sensors can be configured to function independently or simultaneously to sense two or more analytes concurrently, sequentially, and/or randomly (which is inclusive of events that can take place independently in picoseconds, nanoseconds, milliseconds, seconds, or minutes) or in an alternating or overlapping fashion. The two or more sensors of the sensor system can be communicatively coupled to electronics, e.g., a single transmitter or receiver. The two or more sensors of the sensor system can be communicatively coupled to separate, independent electronics.

In one example of a continuous analyte monitoring system, a first, single, sensor is configured to continuously monitor at least a first analyte (e.g., glycose, glycerol, lactate, bilirubin, oxygen, etc.) and a second, different, analyte. In this example, the single sensor may include a single coaxial or planar sensor configured to monitor the at least first analyte and the second analyte. In another example, a first sensor is configured to monitor the first analyte, and a second sensor is configured to continuously monitor a second analyte (e.g., ketones). Each of the first sensor and the second sensor may be planar, substantially planar, or coaxial, or a combination of two or more top, side, or cross-sectional geometries. In one example, each of the first sensor and the second sensor are communicatively coupled to the same sensor electronics and networking elements to continuously monitor and provide feedback to a device, e.g., a mobile device, tablet, laptop, wearable technology (clothing, jewelry, other accessory) or other IoT (internet-of-things) device or combinations of devices. In another example, the first sensor and the second sensor are communicatively coupled to independent sensor electronics and networking elements. Each of the first sensor and the second sensor are positioned in a subject in a subcutaneous layer through a skin layer. In another example, a sensor system is configured as a monolithic sensor body having both the first sensor and the second sensor with their electrodes configured to detect two or more analytes. At least one plurality of electrodes of the sensor system is configured to detect a first analyte, and a second plurality of electrodes is configured to detect a second analyte. The sensor system is positioned in a subject in a subcutaneous layer through a skin layer. In yet another example, a sensor system includes a first sensor and a second sensor, where each sensor of the sensor system includes one or more fiber elements. For example, two or more sensors such as the first sensor and the second sensor may be electrically, mechanically, or otherwise coupled together ex vivo, in vivo, or both. Each of the first sensor and the second sensor of the sensor system is positioned in a subject in a subcutaneous layer through a skin layer.

The multi-analyte sensor device and systems discussed herein may include elements such as on-body wearable devices, wireless communication capabilities, electronics, software, GUI(s), or other elements configured to cause the continuous analyte monitoring systems to continuously monitor analyte levels in a host. Various alerts and actions may be taken in response to this monitoring. As discussed herein, an “on-body” device or wearable device includes devices configured to couple to a host for at least a predetermined period of time via one or more coupling elements including an in-vivo component such as a sensor, and/or adhesives, mechanical elements, electrical elements, magnetic elements, or other combinations of elements.

### Sensing Membrane

In some examples, as shown in FIG. 7, a sensing membrane is disposed over the electroactive surfaces of the continuous multi-analyte sensor 700 and includes one or more domains or layers. In general, the sensing membrane functions to control the flux of a biological fluid there through and/or to protect sensitive regions of the sensor from contamination by the biological fluid, for example. Some electrochemical enzyme-based analyte sensors generally include a sensing membrane that controls the flux of the analyte being measured, protects the electrodes from contamination of the biological fluid, and/or provides an enzyme that catalyzes the reaction of the analyte with a co-factor, for example. See, e.g., U.S. Pat. Appl. Pub. No. 2005/0245799 to Brauker et al. and U.S. Pat. No. 7,497,827 to Brister et al., which are incorporated herein by reference in their entirety.

The sensing membranes of the present disclosure can include any membrane configuration suitable for use with any analyte sensor (such as described in more detail above). In general, the sensing membranes of the present disclosure include one or more domains, all or some of which can be adhered to or deposited on the analyte sensor as is appreciated by a person of ordinary skill in the art. In one example, the sensing membrane generally provides one or more of the following functions: 1) protection of the exposed electrode surface from the biological environment, 2) diffusion resistance (limitation) of the analyte, 3) a catalyst for enabling an enzymatic reaction, 4) limitation or blocking of interfering species, and 5) hydrophilicity at the electrochemically reactive surfaces of the sensor interface, such as described in U.S. Pat. No. 7,497,827 to Brister et al., referenced above. The sensing membranes discussed herein may include one or more adhesive layers positioned in between two adjacent membrane layers. In one example, the one or more adhesive layers can increase robustness and adherence, thus improving the sensing membrane integrity. In various examples, the adhesive layer may include silane groups, polyvinyl alcohol (PVA), glutaraldehyde, or silicone-based or silicone-including materials, or other adhesives or combinations of adhesives.

### Electrode Domain

In some examples, the membrane system comprises an optional electrode domain. The electrode domain is provided to promote and/or enhance an electrochemical reaction between the electroactive surfaces of the working electrode and the reference electrode, and thus the electrode domain is situated more proximal to the electroactive surfaces than the enzyme domain. In some examples, the electrode domain includes a semipermeable coating that maintains a layer of water at the electrochemically reactive surfaces of the sensor, for example, a humectant in a binder material can be employed as an electrode domain; this allows for the full transport of ions in the aqueous environment. The electrode domain can also assist in stabilizing the operation of the sensor by overcoming electrode start-up and drifting problems caused by inadequate electrolyte. The material that forms the electrode domain can also protect against pH-mediated damage that can result from the formation of a large pH gradient due to the electrochemical activity of the electrodes.

In one example, the electrode domain includes a flexible, water-swellable, hydrogel film having a “dry film” thickness of from about 0.05 micron or less to about 20 microns or more. In some examples, the “dry film” thickness is from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. In further examples, the “dry film” thickness is from about 2, 2.5 or 3 microns to about 3.5, 4, 4.5, or 5 microns. “Dry film” thickness refers to the thickness of a cured film cast from a coating formulation by standard coating techniques.

In certain examples, the electrode domain is formed of a curable mixture of a urethane polymer and a hydrophilic polymer. Coatings are formed of a polyurethane polymer having carboxylate functional groups and non-ionic hydrophilic polyether segments, wherein the polyurethane polymer is crosslinked with a water soluble carbodiimide (e.g., 1-ethyl-3-(3-dimethylaminopropyl)carbodiimide (EDC) or polycarbodiimide crosslinker) in the presence of polyvinylpyrrolidone and cured at a moderate temperature of about 50° C.

In some examples, the electrode domain is deposited by spray or dip-coating the electroactive surfaces of the sensor. In further examples, the electrode domain is formed by dip-coating the electroactive surfaces in an electrode solution and curing the domain for a time of from about 15 to about 30 minutes at a temperature of from about 40 to about 55° C. (and can be accomplished under vacuum (e.g., 20 mmHg to 30 mmHg)). In examples wherein dip-coating is used to deposit the electrode domain, a insertion rate of from about 1 to about 3 inches per minute, with a dwell time of from about 0.5 to about 2 minutes, and a withdrawal rate of from about 0.25 to about 2 inches per minute provide a functional coating. However, values outside of those set forth above can be acceptable or even desirable in certain examples, for example, dependent upon viscosity and surface tension as is appreciated by a person of ordinary skill in the art. In one example, the electroactive surfaces of the electrode system are dip-coated one time (one layer) and cured at 50° C. under vacuum for 20 minutes.

As discussed herein, the insertable portion coating composition applied to the insertable portion may have a viscosity of from about 10 centipoise (cP) to about 350 cP. In another example, the insertable portion coating composition applied to the insertable portion has a viscosity from about 20 cP to about 200 cP. In still another example, the insertable portion coating composition applied to the insertable portion has a viscosity from about 30 cP to about 300 cP.

Although an independent electrode domain is described herein, in some examples, sufficient hydrophilicity can be provided in the interference domain and/or enzyme domain (depending on which domain is adjacent to the electroactive surfaces) so as to provide for the full transport of ions in the aqueous environment (e.g. without a distinct electrode domain).

### Non-Mediated System Interference Domain

In some examples, an optional interference domain is provided for non-mediated systems disclosed herein, which generally includes a polymer domain that restricts the flow of one or more interferants to the working electrode. In some examples, the interference domain functions as a molecular sieve that allows analytes and other substances that are to be measured by the electrodes to pass through, while preventing passage of other substances, including interferants such as ascorbate and urea (see U.S. Pat. No. 6,001,067 to Shults). Some known interferants for a glucose-oxidase based electrochemical sensor include acetaminophen, ascorbic acid, bilirubin, cholesterol, creatinine, dopamine, ephedrine, ibuprofen, L-dopa, methyldopa, salicylate, tetracycline, tolazamide, tolbutamide, triglycerides, and uric acid.

Several polymer types that can be utilized as a base material for the interference domain include polyurethanes, polymers having pendant ionic groups (e.g., polyurethane-zwitterion) NAFION™, chitosan, cellulose, or alternating layers of polyallylamine and polyacrylate acid, etc., and polymers having controlled pore size, for example. In one example, the interference domain includes a thin, hydrophobic membrane that is non-swellable and restricts diffusion of low molecular weight species. The interference domain is permeable to relatively low molecular weight substances, such as hydrogen peroxide, but restricts the passage of higher molecular weight substances, including glucose and ascorbic acid. In one example, the interference domain comprises charged species (e.g., polymers with pendent charged groups as disclosed herein) that function to interact with one or more species of the sensing system, such as a cofactor, to reduce or eliminate migration from a domain.

Other systems and methods for reducing or eliminating interference species that can be applied to the membrane system of the present disclosure are described in U.S. Pat. No. 7,816,004 to Muradov et al., U.S. Pat. Appl. Pub. No. 2005/0176136 to Burd et al., U.S. Pat. No. 7,081,195 to Simpson et al., and U.S. Pat. No. 7,715,893 to Kamath et al. In some alternate examples, a distinct interference domain is not included.

In one example, the interference domain is deposited onto the electrode domain (or directly onto the electroactive surfaces when a distinct electrode domain is not included) for a dry film domain thickness of from about 0.05 micron or less to about 20 microns or more. In other examples, the dry film domain thickness is from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. In further examples, the dry film domain thickness is from about 2, 2.5 or 3 microns to about 3.5, 4, 4.5, or 5 microns. Thicker membranes can also be useful, but, in some examples, thinner membranes have a lower impact on the rate of diffusion of the electroactive species from the enzyme domain to the electrodes.

As discussed herein, when two or more sensors are employed in a sensor system, each sensor optionally includes an interference domain configured to prevent the same interferent(s) from permeating the membrane. In another example, when two or more sensors are employed in a sensor system, each sensor optionally includes an interference domain configured to prevent the different, or overlapping but also different, interferent(s) from permeating the membrane.

### Mediated System—Interference Domains

Some second generation electrochemical analyte sensor technologies (2^{nd}-gen) leverage immobilized redox mediators to reduce the overpotential required to detect an analyte. This reduction can be significant in contrast to the typical operating potentials for first generation electrochemical analyte sensors (1^{st}-gen, e.g., those operating on the principle of hydrogen peroxide detection on a catalytic metal surface). As an example, 2^{nd}-gen analyte sensor may be biased between +0.0V and +0.3V verses +0.5 to +0.8V for 1^{st}-gen sensors. However, despite this reduction in operating potential and reduction in susceptibility to electroactive interference from endogenous and pharmacologic agents, these 2^{nd}-generation sensors can still succumb to the undue effect of residual interference.

For example, exemplary 2^{nd}-generation analyte sensors utilize polymer-bound covalently-bound redox mediators (e.g., polyvinyl imidazole (PVI)-Os(4,4′-dimethyl-2,2′-bipyridine)2C1]+/2+) that reduce the overpotential required for the enzymatic detection of a target analyte. An example of such mediator-based sensors include the systems where undue signal influence arising from the presence of co-circulating endogenous electroactive species can result as evidenced by product labeling warnings regarding large doses of ascorbic acid/ascorbate ion (i.e., Vitamin C), possibly resulting in false hyperglycemia alerts and the like. While charge-selective membranes or further reduction in overpotential may mitigate such interference effects, it may result in a material impact to sensitivity and signal-to-noise figures of merit. Accordingly, at present, mediated electrochemical analyte sensing systems continue to exhibit undue signal influence from endogenous metabolites, such as ascorbic acid.

Thus, the present disclosure includes mediated system interference domains developed for 2^{nd}-gen sensor systems, whether they be continuous glucose monitoring or multi-analyte monitoring, e.g., ketone-glucose monitoring, the mediated system interference domains comprising one or more oxidase enzymes, which elicit the enzymatic degradation of an interfering metabolite, or ensemble of metabolites, into a peroxide product, for example hydrogen peroxide. The present disclosure provides domains comprising oxidase enzymes alone or in combination with any of the conventional membranes (electrode, enzyme, resistance domains/layers) used with an indwelling 2^{nd}-generation (e.g., mediated) analyte sensor. Exemplary oxidase enzymes include, for example, ascorbate oxidase or urate oxidase that are configured to catalytically convert an undesired interfering species (e.g., ascorbic acid, uric acid) to a hydrogen peroxide product, which manifests significantly less influence at the bias voltages/overpotentials conventionally applied in 2^{nd}-generation sensing systems. This conversion provides reduced overall concentration of the interfering species at the electrode surface (e.g., trading the flux of the interfering species with the flux of hydrogen peroxide), which provides less detrimental effect to the sensed signal than otherwise would be possible in the presence of the interfering species. The mediated system interference domain can be used alone or in combination with other interference domains, interference membranes, or interference domains, said other interference domains, interference membranes, or interference domains can comprise the same polymer(s) matrix, for example, without the one or more oxidase enzymes, peroxidase or catalase.

In some examples of the mediated system interference domain, the oxidase enzymes can be combined with one or more peroxidase or peroxidase-like enzymes (e.g., horseradish peroxidase, catalase) to further cleave the generated hydrogen peroxide product from the oxidase enzyme(s), thereby rendering peroxide electroactive agent inert and unable to undergo a redox reaction at the electrode surface. The present disclosure includes deployment of the mediated system interference domain in one or more of the electrode domain, the enzyme domain, the resistance domain and the interference membrane. The present disclosure includes deployment of the mediated system interference domain in one or more of the electrode domain, the enzyme domain, the resistance domain and the interference domain.

Thus, in one example, using an exemplary ketone/glucose multianalyte sensor system, can comprise the mediated system interference domain comprising at least one of ascorbate oxidase, urate oxidase, horseradish peroxidase, or catalase is present in an enzyme domain comprising a dehydrogenase enzyme (e.g., beta-hydroxybutyrate dehydrogenase, NADH-acting enzyme (e.g., diaphorase, NAD(P)H dehydrogenase), redox polymer (e.g., PVI-Os(bpy)2Cl), optionally a co-factor (if needed, e.g., NAD+, NADP+) and be optionally crosslinked, e.g., using PEG-DGE, CDI or polycarbodiimide crosslinkers. A resistance domain of a biocompatible material or blend of hydrophobic/hydrophilic polymer, for example PVP/PEG-DGE) can be applied over the enzyme domain/mediated system interference domain.

In another example, using an exemplary ketone/glucose multianalyte sensor system, the mediated system interference domain comprising at least one of ascorbate oxidase, urate oxidase, horseradish peroxidase, or catalase is present in a resistance domain comprising of a biocompatible material or blend of hydrophobic/hydrophilic polymer, for example PVP/PEG-DGE). A separate enzyme domain can be positioned proximal to the electrode and adjacent the mediated system interference domain present in the resistance domain, the enzyme domain comprising dehydrogenase enzyme (e.g., beta-hydroxybutyrate dehydrogenase, NADH-acting enzyme (e.g., diaphorase, NAD(P)H dehydrogenase), redox polymer (e.g., PVI-Os(bpy)2Cl), optionally a co-factor (if needed, e.g., NAD+, NADP+) and be optionally crosslinked, e.g., using PEG-DGE or polycarbodiimide crosslinker.

In another example, using an exemplary ketone/glucose multianalyte sensor system, the mediated system interference domain comprising at least one of ascorbate oxidase, urate oxidase, horseradish peroxidase, or catalase is present between an enzyme domain and at least one electrode surface. A resistance domain of a biocompatible material or blend of hydrophobic/hydrophilic polymer, for example PVP/PEG-DGE, can be applied over the enzyme domain.

In other examples, an exemplary ketone or ketone/glucose multianalyte sensor system that is without a mediator, is provided, as discussed further herein. In one example, an exemplary ketone or ketone/glucose multianalyte sensor system that is without a metal-based mediator, e.g., osmium complexes of biimidazole and/or imidazole ligands, is provided. In one example, an exemplary ketone or ketone/glucose multianalyte sensor system that is without a metal-based mediator, e.g., osmium complexes of biimidazole and/or imidazole ligands, configured to provide amperometric signal at an applied voltage of greater than +0.2 V, greater than or equal to +0.3 V, greater than or equal to +0.4 V, greater than or equal to +0.5 V, or greater than or equal to +0.6 V, is provided. In one example, an exemplary ketone or ketone/glucose multianalyte sensor system that is without a metal-based mediator and includes an interference layer, is provided. In one example, an exemplary ketone or ketone/glucose multianalyte sensor system that is without a metal-based mediator, e.g., osmium complexes of biimidazole and/or imidazole ligands, configured to provide amperometric signal at an applied voltage of greater than +0.2 V, greater than or equal to +0.3 V, greater than or equal to +0.4 V, greater than or equal to +0.5 V, or greater than or equal to +0.6 V, and includes an interference layer, is provided.

### Transducing Element Domain

In one example, the membrane system further includes a transducing element domain, for example an enzyme, RNA, DNA, aptamer, binding protein, etc., disposed more distally from the electroactive surfaces than the interference domain (or electrode domain when a distinct interference is not included). In some examples, the transducing element domain is directly deposited onto the electroactive surfaces (when neither an electrode nor interference domain is included). In one example, the transducing element domain provides an enzyme to catalyze the reaction of the analyte and its co-reactant, as described in more detail below. In some examples, the transducing element domain includes glucose oxidase; however other oxidases, for example, galactose oxidase or uricase, can also be used.

For enzyme-based electrochemical sensors to perform effectively and accurately, the sensor's response is limited by neither enzyme activity nor by co-reactant concentration. Enzymes, including glucose oxidase, can be subject to deactivation as a function of time even in ambient conditions, and this behavior is compensated for in forming the enzyme domain. In some examples, the enzyme domain is constructed of aqueous dispersions of colloidal polyurethane polymers including the enzyme. However, in alternate examples the enzyme domain is constructed from an oxygen-enhancing material, for example, at least one of silicone or fluorocarbon, in order to provide a supply of excess oxygen to ensure that oxygen does not limit the sensing reaction. In some examples, the enzyme is immobilized within the enzyme domain. See U.S. Pat. No. 7,379,765 Petisce et al.

In one example, the transducing element domain is deposited onto the interference domain for a “dry film” domain thickness of from about 0.05 micron or less to about 20 microns or more. In other examples, the dry film domain thickness is from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. In further examples, the dry film domain thickness is from about 2, 2.5 or 3 microns to about 3.5, 4, 4.5, or 5 microns. “Dry film” thickness refers to the thickness of a film cast from a coating formulation by standard coating techniques and includes post-curing of the film.

However, in some examples, the transducing element domain is deposited onto the electrode domain or directly onto the electroactive surfaces. In some examples, the transducing element domain is deposited by spray or dip-coating, slot die coating, 3D printing, pico jet printing, piezo inkjet printing, and the like. In further examples, the transducing element domain is formed by dip-coating the electrode domain into an transducing element domain solution and curing the transducing element domain for from about 15 to about 30 minutes at a temperature of from about 40 to about 550° C. (and can be accomplished under vacuum (e.g., 20 to 30 mmHg)). In examples wherein dip-coating is used to deposit the transducing element domain at room temperature, a insertion rate of from about 1 inch per minute to about 3 inches per minute, with a dwell time of from about 0.5 minutes to about 2 minutes, and a withdrawal rate of from about 0.25 inch per minute to about 2 inches per minute provide a functional coating. However, values outside of those set forth above can be acceptable or even desirable in certain examples, for example, dependent upon viscosity and surface tension as is appreciated by a person of ordinary skill in the art. In one example, the transducing element domain is formed by dip-coating two times (namely, forming two layers) in a coating solution and curing at 50° C. under vacuum for 20 minutes. However, in some examples, the transducing element domain can be formed by dip-coating and/or spray-coating one or more layers at a predetermined concentration of the coating solution, insertion rate, dwell time, withdrawal rate, and/or desired thickness. In yet another example, the transducing element layer is formed from multiple interlayers deposited by self-assembling-monolayers (SAMs) which are usually formed by immersion in the solution that facilitates the surface chemistry. A substrate may be disposed in this solution for a period of time from about 30 minutes to about 24 hours to form the desired transducing element layer to a predetermined thickness. In another example, the substrate may be disposed in this solution for a period of time from about 1 hour to about 18 hours. In another example, the substrate may be disposed in this solution for a period of time from about 3 hours to about 12 hours.

In still other example, the transducing element layer is formed from multiple interlayers. One or more interlayers of the transducing element layer may be varied alone or in combination in various aspects such as chemistry (composition), thickness, or other mechanical, electrical, biological, or other material properties to achieve a target electron mobility or a range of electron mobility through each interlayer.

### Resistance Domain

In one example, the membrane system includes a resistance domain disposed more distal from the electroactive surfaces than the enzyme domain. Although the following description is directed to a resistance domain for a glucose sensor, the resistance domain can be modified for facilitating detection of the concentrations(s) of other analytes and co-reactants as well. In one example, the resistance domain is configured to control the flux of oxygen through the membrane. In another example, the resistance domain is configured to control the flux of an analyte or co-reactant other than oxygen through the membrane. In yet another example, the resistance domain is configured to control the flux of two or more analytes through the membrane.

An immobilized enzyme-based glucose sensor employing oxygen as co-reactant is supplied with oxygen in non-rate-limiting excess in order for the sensor to respond linearly to changes in glucose concentration, while not responding to changes in oxygen concentration. Specifically, when a glucose-monitoring reaction is oxygen limited, linearity is not achieved above minimal concentrations of glucose. Without a semipermeable membrane situated over the enzyme domain to control the flux of glucose and oxygen, a linear response to glucose levels can be obtained only for glucose concentrations of up to about 40 mg/dL. However, in a clinical setting, a linear response to glucose levels is desirable up to at least about 400 mg/dL.

In one example, the resistance domain includes a semi-permeable membrane that controls the flux of oxygen and glucose to the underlying enzyme domain, rendering oxygen in a non-rate-limiting excess. As a result, the upper limit of linearity of glucose measurement is extended to a much higher value than that which is achieved without the resistance domain. In one example, the resistance domain exhibits an oxygen to glucose permeability ratio of from about 50:1 or less to about 400:1 or more. In further examples, the oxygen to glucose permeability ratio is about 200:1.

In alternate examples, a lower ratio of oxygen-to-glucose can be sufficient to provide excess oxygen by using a high oxygen solubility domain (for example, a silicone or fluorocarbon-based material or domain) to enhance the supply/transport of oxygen to the transducing element domain. If more oxygen is supplied to the enzyme, then more glucose can also be supplied to the transducing element without creating an oxygen rate-limiting excess. In alternate examples, the resistance domain is formed from a silicone composition, such as is described in U.S. Pat. Appl. Pub. No. 2005/0090607 to Tapsak et al.

In one example, the resistance domain includes a polyurethane membrane with both hydrophilic and hydrophobic regions. The hydrophilic and hydrophobic regions may be used in combination to control the diffusion of an analyte or analytes (e.g., glucose, oxygen, ketones, lactate, uric acid, etc.) to an analyte sensor. A suitable hydrophobic polymer component is a polyurethane, or polyurethane urea. Polyurethane is a polymer produced by the condensation reaction of a diisocyanate and a difunctional hydroxyl-containing material. A polyurethane urea is a polymer produced by the condensation reaction of a diisocyanate and a difunctional amine-containing material. In polyurethanes and polyurethane urea polymers, either of the hard segment or soft segment can comprise a plurality of distinct chemical structures, e.g., a soft segment can comprise hydrophobic and hydrophilic segments.

Example diisocyanates useful as the hard segment component of polyurethane or polyurethane urea polymers of the present disclosure include aliphatic diisocyanates containing from about 4 to about 8 methylene units. Diisocyanates containing cycloaliphatic moieties can also be useful in the preparation of the polymer and copolymer components of the membranes of the present disclosure. The material that forms the basis of the hydrophobic matrix of the resistance domain may be selected to exhibit sufficient permeability to allow relevant compounds to pass through it, for example, to allow an oxygen molecule to pass through the membrane from the sample under examination in order to reach the active enzyme or electrochemical electrodes. Examples of materials which can be used to make non-polyurethane type membranes include vinyl polymers (including polyvinylimidazole and poly vinylpyridine), polyethers, polyesters, polyamides, inorganic polymers such as polysiloxanes and polycarbosiloxanes, natural polymers such as cellulosic and protein-based materials, and mixtures or combinations thereof. In some examples, these non-polyurethane type membranes include a crosslinking agent in addition to the base polymer, in order to improve mechanical properties and/or tune mass transport of analyte or other species. In some examples, the resistance domain may polyvinyl butyral (PVB). In other examples, the base polymer can be a segmented block copolymer. In another example, the hard segments may be from about 15 wt. % to about 75 wt. %. In yet another example, the hard segments may be from about 25 wt. % to about 55 wt. %. In yet another example, the hard segments may be from about 35 wt. % to about 45 wt. %. For example, the base polymer can comprise polyurethane and/or polyurea segments and one or more of polycarbonate, polydimethylsiloxane (PDMS), polyether, fluoro-modified segments, perfluoropolyols, or polyester segments. In other examples, the base polymer can be a polyurethane copolymer chosen from the group including a polyether-urethane-urea, polycarbonate urethane, polyether-urethane, polyester-urethane, and/or copolymers thereof.

In one example, the hydrophilic polymer component of the resistance domain is polyethylene oxide. For example, one useful hydrophobic-hydrophilic copolymer component is a polyurethane polymer that includes from about 1 wt. % to about 50 wt. % polyethylene oxide (PEO). In one example, the resistance domain includes 5 wt. % to about 30 wt. % polyethylene oxide (PEO). In another example, the resistance domain includes from about 10 wt. % to about 40 wt. % PEO. The polyethylene oxide portions of the copolymer are thermodynamically driven to separate from the hydrophobic portions of the copolymer and the hydrophobic polymer component. The polyethylene oxide-based soft segment portion of the copolymer used to form the final blend affects the water pick-up and subsequent glucose permeability of the membrane.

In one example, one or more of NBDI, IPDI, TDI, MPDI, HMDI, MDI, 1,3-H6XDI, 1,4-H6XDI, CHDI, PPDI, TODI, or HDI, diisocyanates are used to form various polyurethanes and polyurethane ureas for the resistance domain and/or other sensor domains. In one example, the polyurethanes and polyurethane ureas have soft segments that are aliphatic or amphiphilic. In one example, the soft segment is comprised diol, diamine, diester, or dicarbonate. In one example, the soft segment is comprised of a plurality of two or more of diol, diamine, diester, or dicarbonate.

In one example, one or more of NBDI, IPDI, TDI, MPDI, HMDI, MDI, 1,3-H6XDI, 1,4-H6XDI, CHDI, PPDI, TODI, HDI, is reacted with one or more dicarbonates, polyethers, polyesters, polyalkyl-diols or polyakyl-diamines.

In one example, one or more of NBDI, IPDI, TDI, MPDI, HMDI, MDI, 1,3-H6XDI, 1,4-H6XDI, CHDI, PPDI, TODI, HDI, is reacted with a C5 or C6 dicarbonate, for example U90 OXYMER™, polyhexmethylene carbonate glycol (PHA). In one example, NBDI, IPDI, TDI, MPDI, HMDI, MDI, 1,3-H6XDI, 1,4-H6XDI, CHDI, PPDI, TODI, HDI, or mixtures thereof is reacted with a C5 or C6 dicarbonate, for example U90 OXYMER™ and one or more polyethers, polyesters, polyalkyl-diols or polyakyl-diamines. In one example, the dicarbonate is sterically branched to increase the Tg of the soft segment, for example to provide a Tg around body temperature.

In one example, one or more of hard segment diisocyantes of NBDI, IPDI, TDI, MPDI, HMDI, MDI, 1,3-H6XDI, 1,4-H6XDI, CHDI, PPDI, TODI, HDI, is reacted with a polyether, for example, one or more of polytetramethylene oxide (PTMO), polypropylene oxide (PPO), polyethylene glycol (PEG), polybutadiene diol (PBU) alone or in combination with polydimethylpolysiloxane (PDMS). In one example, the same polyether of different molecular weight (Mw) is used. In one example, two or more polyethers of the same or different Mw are used. In one example, one or more polyethers of the same or different Mw are used in combination with one or more PDMS polymers having the same or different Mw. While not be held to any particular theory, as the molecular weight of the soft segment decreases, phase mixing of different soft segment components increases. In one example, it has been observed that high molecular weight of the soft segment provides for the formation of rich phases, likely due to entropic contributions, among other things.

In one example, one or more of hard segment diisocyanates of NBDI, IPDI, TDI, MPDI, HMDI, MDI, 1,3-H6XDI, 1,4-H6XDI, CHDI, PPDI, TODI, HDI, is reacted with one or more polyesters, for example, polyethylene adipate glycol (PEA), polyteramethylene adipate glycol (PBA), alone or in combination with one or more polyethers, polyalkyl-diols or polyakyl-diamines.

In one example, NBDI, IPDI, TDI, MPDI, HMDI, MDI, 1,3-H6XDI, 1,4-H6XDI, CHDI, PPDI, TODI, HDI, or mixtures thereof is reacted with one or more polyalkyl-diols, alone or in combination with one or more polycarbonates, polyethers, polyesters, or polyakyl-diamines.

In one example, the resistance domain described above is deposited directly onto the electrode surface or onto the enzyme domain in one or more layers to yield a resistance domain thickness of from about 0.05 micron or less to about 20 microns or more. In another example, the total resistance domain thickness is from about 0.05, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns. In another example, the total resistance domain thickness is from about 2, 2.5, or 3 microns to about 3.5, 4, 4.5, or 5 microns. In some examples, the resistance domain is deposited onto the enzyme domain by spray coating or dip-coating, slot die coating, 3D printing, pico jet printing, piezo inkjet printing. In certain examples, spray coating is the deposition technique. The spraying process atomizes and mists the solution, and therefore most or all of the solvent is evaporated prior to the coating material settling on the underlying domain, thereby minimizing contact of the solvent with the enzyme. One additional advantage of spray-coating the resistance domain as described in the present disclosure includes formation of a membrane system that substantially blocks or resists ascorbate (a known electrochemical interferant in hydrogen peroxide-measuring glucose sensors). While not wishing to be bound by theory, it is believed that during the process of depositing the resistance domain as described in the present disclosure, a structural morphology is formed, characterized in that ascorbate does not substantially permeate there through.

### Heterocyclic Resistance Domains and CoFactor Immobilization or Retention Domains

In one example, the cofactor and enzyme are present in a domain, e.g., enzyme and/or resistance domain comprising poly vinylpyridine, poly vinylpyridine-co-styrene, poly vinylpyridine copolymers with vinyl and (meth)acrylic monomers, poly(styrene-co-acrylonitrile), polyvinylimidazoles, or polyvinylimidazole copolymers with vinyl and (meth)acrylic monomers and/or provided as a layer adjacent the electrode domain or the electrode surface. As used herein, “poly vinylpyridine” encompasses poly 2-vinylpyridine, 3-vinylpryidine, 3-vinylpryidine, and alkyl substituted derivatives thereof. Blends and/or graphs of the above polymers can be used. Blends and/or graphs of the above polymers with chitosan, amphiphilic or aliphatic polyurethanes or polyurethane urea, polyols (PEG, PTMO etc.), or zwitterionic polymers can be used. In some examples, polystyrene copolymers with vinyl monomers containing electron withdrawing groups, such as nitrile can be used. In some examples, vinyl polymers with benzene and nitrile functional groups can be used.

In one example, the cofactor and enzyme are present in a domain, e.g., enzyme and/or resistance domain comprising an at least partially cross-linked poly(4-vinylpyridine), polyvinyl pyridine-co-styrene, polyvinyl pyridine copolymers with vinyl and (meth)acrylic monomers, poly(styrene-co-acrylonitrile), polyvinylimidazoles, or polyvinyl imidazole copolymers with vinyl and (meth)acrylic monomers are used as the resistance domain and/or provided as a layer adjacent the electrode domain or the electrode surface. In one example, poly(4-vinylpyridine), polyvinyl pyridine-co-styrene, polyvinyl pyridine copolymers with vinyl and (meth)acrylic monomers, poly(styrene-co-acrylonitrile), polyvinylimidazoles, or polyvinyl imidazole copolymers with vinyl and (meth)acrylic monomers, with or without cross-linking provide for immobilization or retention of one or more cofactors in the resistance domain. In one example, immobilization or retention of the cofactor is via covalent bonding with a function group of the polymer. In another example, immobilization or retention of the cofactor is via a non-covalent interaction, e.g., via equilibrium with a function group of the polymer.

Examples of cofactor immobilization via non-covalent interaction with polymers includes NAD+, for example, with cationic polymers (chitosan, quaternized PVPy, poly zwitterionic polymers, etc.), and/or polymers containing boronic acid functional groups. Thus, in one example, the cofactor and enzyme are present in a domain, e.g., enzyme and/or resistance domain comprising polymers with pendant boronic acid groups that provide for strong dynamic covalent bonding with diol functionalities on cofactors with diol functionality, e.g. NAD, at certain pH, allowing for NAD(H) immobilization or retention. Thus, in one example, the ribose ring structure containing 1,2 diols of the NADH and NAD+ structure are used to associate and/or bind to one or more boronic acid functional groups via at least the covalent interactions as shown in Scheme 1a and Scheme 1b for the NADH form. Similar covalent interactions are envisaged for the NAD+ form.

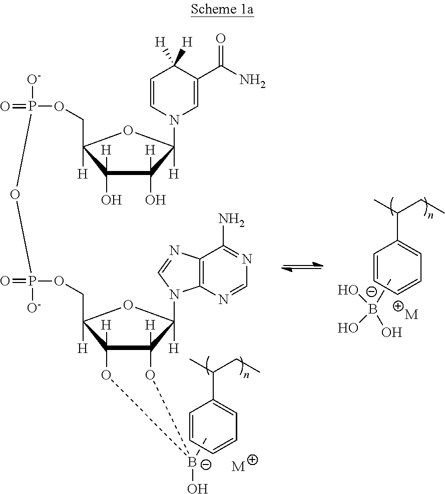

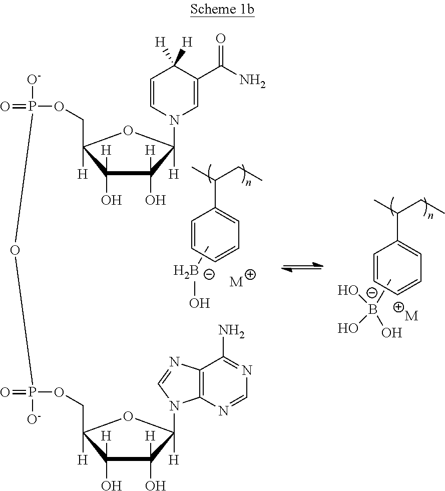

In one example, boronic acid polymer structure and coating solution pH are adjusted to provide sufficient association of the NAD/NADH structure to reduce or eliminate migration from the polymeric membrane. In one example, boronic acid polymers include styrenic polymers, styrenic copolymers (e.g., with acrylic, acrylate, acrylamide, olefinic, cyclic olefinic), naphthyl, anthracenyl polymers and copolymers thereof. In one example, the boronic acid polymer is at least partially crosslinked.

In some examples, the domain is configured to repulse cofactor, for example, the RL functions to “repel” NAD(H) and not let it through, thus attenuating its migration out of the EZL.

In another example, the NAD is tethered to a domain or electrode surface. In another example, the NAD is directly tethered to a domain or directly coupled to an electrode surface. In another example, the NAD is coupled to the electrode surface with an electron transfer agent. In one example, the free amine of the adenine group of the NAD(H) is extended to have an alkyl chain with a primary amine to provide for EDC or (sulfo-)NHS coupling chemistry with a —COOH group on a mediator as shown in Scheme 3, that depicts the modified NAD+ cofactor with an extended free —NH2 coupled to one of the —COOH groups in a PQQ (pyrroloquinoline quinone) mediator.

In another example, the free amine of the adenine group of the NAD(H) is extended to have an alkyl chain with a primary amine to provide for EDC or (sulfo-)NHS coupling chemistry with a HBDH enzyme.

In one example, the modified NAD+ cofactor has an extended free —NH2 which can be readily crosslinked to one of the —COOH groups in a PQQ (pyrroloquinoline quinone) mediator. This PQQ mediator has another —COOH group that can then be crosslinked to a polymer backbone, enzyme, or directly onto the electrode surface.

In one example, the cofactor and enzyme are present in a domain, e.g., enzyme and/or resistance domain comprising an amphiphilic polyurethane or polyurethane urea polymer as disclosed above for the biointerface/drug releasing layer, the aliphatic polyurethane or polyurethane urea having a hard segment content of about 20-40 wt %, a polysiloxane segment of about 10-30 wt %, and a polyglycol segment of about 15-40 wt %. In one example, the amphiphilic polyurethane or polyurethane urea also includes polyvinyl pyrrolidone polymer of 0-25 wt %. In one example, the amphiphilic polyurethane or polyurethane urea also includes polyvinyl pyridine or an alkylated or polyol substituted pyridine polymer of 0-25 wt %. Such amphiphilic polyurethane or polyurethane urea polymer resistance domains provided acceptable sensitivity and greater than two week stability. In one example, the amphiphilic polyurethane or polyurethane urea polymer is at least partially crosslinked. Such amphiphilic polyurethane or polyurethane urea polymer resistance domains provided sensitivity and stability compatible with the PVPy resistance domains discussed above.

In one example, the cofactor and enzyme are present in a domain, e.g., enzyme and/or resistance domain comprising an aliphatic polyurethane or polyurethane urea polymer comprising a hard segment content of about 40-60 wt %, polytetrahydrofuran (PTMO) segments of about 15-50 wt. %, and a polysiloxane segment of about 5-30 wt %. In one example, the aliphatic polyurethane or polyurethane urea polymer is at least partially crosslinked. In one example, polycarbodiimide crosslinkers are used. Such aliphatic polyurethane or polyurethane urea resistance domains showed sensitivity and stability less than the amphiphilic polyurethane or polyurethane urea based resistance domains.

In one example, the cofactor and enzyme are present in a domain, e.g., enzyme and/or resistance domain comprising a block copolymer obtained by polycondensation of a carboxylic acid polyamide (e.g., PA6, PA11, PA12) with a polyether (e.g., polytetramethylene glycol, polyethylene glycol, PEG, polytetrahydrofuran PTMO). In one example, the block copolymer obtained by polycondensation of a carboxylic acid polyamide with a polyether can be at least partially crosslinked.

In one example, the cofactor and enzyme are present in a domain, e.g., enzyme and/or resistance domain, comprising a polyvinyl pyridine-poly(ethylene glycol) diglycidyl ether (PVPy-PEG-DGE) matrix. In one example, the mass ratio of PEG-DGE to PVPy is about 1-10 wt. %. In one example, polyvinyl pyridine-PEG-DGE matrix is at least partially crosslinked.

In one example, the cofactor and enzyme are present in a domain, e.g., enzyme and/or resistance domain, comprising a water dispersible polyurethane-zwitterion polymer crosslinked with a carbodiimide or polycarbodiimide. Examples of such domains include those disclosed in U.S. Pat. Appl. Pub. No. 2017/0191955, U.S. Pat. Appl. Pub. No. 2017/0188922, and U.S. Pat. No. 11,112,377B2, the disclosures of which are incorporated herein by reference. In one example, the domain comprises an enzyme and a polymer comprising polyurethane and/or polyurea segments and one or more zwitterionic repeating units. In one example, the domain comprises an enzyme and a blend of a polyurethane base polymer and polyvinylpyrrolidone. In some examples, the enzyme domains are formed of a polyurethane urea having carboxyl betaine groups incorporated in the polymer and non-ionic hydrophilic polyethylene oxide segments, wherein the polyurethane urea polymer is dissolved in an organic or non-organic solvent system according to a pre-determined coating formulation, and is optionally crosslinked and/or cured. The above described domain can be from 0.01 m to about 250 m thick.

Depending upon the example, the resistance domain(s) discussed herein can be formed by any number of methods, for example, but not limited to, dip-coating or spray-coating any layer or plurality of layers, depending upon the concentration of the solution, insertion rate, dwell time, withdrawal rate, and/or the desired thickness of the resulting film, or other factors or combinations of factors.

Advantageously, sensors with the membrane system of the present disclosure, including an electrode domain and/or interference domain, an enzyme domain, and a resistance domain, provide stable signal response to increasing glucose levels of from about 40 to about 400 mg/dL, and sustained function (at least 90% signal strength) even at low oxygen levels (for example, at about 0.6 mg/L O₂). While not wishing to be bound by theory, it is believed that the resistance domain provides sufficient resistivity, or the enzyme domain provides sufficient enzyme, such that oxygen limitations are seen at a much lower concentration of oxygen as compared to prior art sensors.

In one example, a sensor signal has a current in the picoAmp range, which is described in more detail elsewhere herein. However, the ability to produce a signal with a current in the picoAmp range can be dependent upon a combination of factors, including the electronic circuitry design (e.g., A/D converter, bit resolution, and the like), the membrane system (e.g., permeability of the analyte through the resistance domain, enzyme concentration, and/or electrolyte availability to the electrochemical reaction at the electrodes), and the exposed surface area of the working electrode. For example, the resistance domain can be designed to be more or less restrictive to the analyte depending upon to the design of the electronic circuitry, membrane system, and/or exposed electroactive surface area of the working electrode.

Accordingly, in one example, the membrane system is designed with a sensitivity of from about 1 pA/mg/dL to about 100 pA/mg/dL. In other examples, the sensitivity is from about 5 pA/mg/dL to 25 pA/mg/dL. In further examples, the sensitivity is from about 4 to about 7 pA/mg/dL. While not wishing to be bound by any particular theory, it is believed that membrane systems designed with a sensitivity in the above ranges permit measurement of the analyte signal in low analyte and/or low oxygen situations. Namely, conventional analyte sensors have shown reduced measurement accuracy in low analyte ranges due to lower availability of the analyte to the sensor and/or have shown increased signal noise in high analyte ranges due to insufficient oxygen necessary to react with the amount of analyte being measured. While not wishing to be bound by theory, it is believed that the membrane systems of the present disclosure, in combination with the electronic circuitry design and exposed electrochemical reactive surface area design, support measurement of the analyte in the picoAmp range, which enables an improved level of resolution and accuracy in both low and high analyte ranges not seen in the prior art.

Although sensors of some examples described herein include an optional interference domain in order to block or reduce one or more interferants, sensors with the membrane system of the present disclosure, including an electrode domain, an enzyme domain, and a resistance domain, have been shown to inhibit ascorbate without an additional interference domain. Namely, the membrane system of the present disclosure, including an electrode domain, an enzyme domain, and a resistance domain, has been shown to be substantially non-responsive to ascorbate in physiologically acceptable ranges. While not wishing to be bound by theory, it is believed that the process of depositing the resistance domain by spray coating, as described herein, results in a structural morphology that is substantially resistance resistant to ascorbate.

### Interference-Free Membrane Systems

In general, it is believed that appropriate solvents and/or deposition methods can be chosen for one or more of the domains of the membrane system that form one or more transitional domains such that interferants do not substantially permeate therethrough. Thus, sensors can be built without distinct or deposited interference domains, which are non-responsive to interferants. While not wishing to be bound by theory, it is believed that a simplified multilayer membrane system, more robust multilayer manufacturing process, and reduced variability caused by the thickness and associated oxygen and glucose sensitivity of the deposited micron-thin interference domain can be provided. Additionally, the optional polymer-based interference domain, which usually inhibits hydrogen peroxide diffusion, is eliminated, thereby enhancing the amount of hydrogen peroxide that passes through the membrane system. In other examples, the interference domain can be configured to block or reduce the diffusion of one or more interfering species, including H₂O₂, acetaminophen, or other interferents or combinations of interferents.

### Oxygen Conduit

As described above, some sensors employ transducing element within the membrane system through which the host's bodily fluid passes and in which the analytes (for example, glucose, ketone) within the bodily fluid reacts in the presence of a co-reactant (for example, oxygen) to generate a product(s). The product is then measured using electrochemical methods, and thus the output of an electrode system functions as a measure of the analyte. For example, when the sensor is a glucose oxidase based glucose sensor, the species measured at the working electrode is H₂O₂. An enzyme, glucose oxidase, catalyzes the conversion of oxygen and glucose to hydrogen peroxide and gluconate according to the following reaction:

Glucose+O₂→Gluconate+H₂O₂.

Because for each glucose molecule reacted there is a proportional change in the product, H₂O₂, one can monitor the change in H₂O₂ to determine glucose concentration. Oxidation of H₂O₂ by the working electrode is balanced by reduction of ambient oxygen, enzyme generated H₂O₂ and other reducible species at a counter electrode, for example. See Fraser, D. M., “An Introduction to In vivo Biosensing: Progress and Problems.” In “Biosensors and the Body,” D. M. Fraser, ed., 1997, pp. 1-56 John Wiley and Sons, New York.

In vivo, glucose concentration is generally about one hundred times or more than that of the oxygen concentration. Consequently, oxygen is a limiting reactant in the electrochemical reaction, and when insufficient oxygen is provided to the sensor, the sensor is unable to accurately measure glucose concentration. Thus, depressed sensor function or inaccuracy is believed to be a result of problems in availability of oxygen to the enzyme and/or electroactive surface(s).

Accordingly, in an alternate example, an oxygen conduit (for example, a high oxygen solubility domain formed from silicone or fluorochemicals or perfluorocarbon compound) is provided that extends from the ex vivo portion of the sensor to the in vivo portion of the sensor to increase oxygen availability to the enzyme. The oxygen conduit can be formed as a part of the coating (insulating) material or can be a separate conduit associated with the assembly of wire(s) that forms the sensor.

In some examples, one or more domains of the sensing membranes are formed from materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, poly(ethylene oxide), poly(propylene oxide) and copolymers and blends thereof, polysulfones and block copolymers thereof including, for example, di-block, tri-block, alternating, random and graft copolymers. U.S. Pat. Appl. Pub. No. 2005/0245799 to Brauker et al., which is incorporated herein by reference in its entirety, describes biointerface and sensing membrane configurations and materials that may be applied to the presently disclosed sensor.

The sensing membrane can be deposited on the electroactive surfaces of the electrode material using known thin or thick film techniques (for example, spraying, electro-depositing, dipping, or the like). It is noted that the sensing membrane that surrounds the working electrode does not have to be the same structure as the sensing membrane that surrounds a reference electrode, etc. For example, the transducing element domain deposited over the working electrode does not necessarily need to be deposited over the reference and/or counter electrodes.

In one example, the sensor is an enzyme-based electrochemical sensor, wherein the working electrode measures the hydrogen peroxide produced by the enzyme catalyzed reaction of glucose being detected and creates a measurable electronic current (for example, detection of glucose utilizing glucose oxidase produces hydrogen peroxide as a by-product, H₂O₂ reacts with the surface of the working electrode producing two protons (2H⁺), two electrons (2e⁻) and one molecule of oxygen (O₂) which produces the electronic current being detected), such as described in more detail above and as is appreciated by a person of ordinary skill in the art. In some examples, one or more potentiostats are employed to monitor the electrochemical reaction at the electroactive surface of the working electrode(s). The potentiostat applies a constant potential to the working electrode and its associated reference electrode to determine the current produced at the working electrode. The current that is produced at the working electrode (and flows through the circuitry to the counter electrode) is substantially proportional to the amount of H₂O₂ that diffuses to the working electrode. The output signal is typically a raw data stream, e.g., a raw signal processed by algorithms prior to display of values, that is used to provide a useful value of the measured analyte concentration in a host to the host or doctor, for example.

Some alternate analyte sensors that can benefit from the systems and methods of the present disclosure include U.S. Pat. No. 5,711,861 to Ward et al., U.S. Pat. No. 6,642,15 to Vachon et al., U.S. Pat. No. 6,654,625 to Say et al., U.S. Pat. No. 6,565,509 to Say et al., U.S. Pat. No. 6,514,718 to Heller, U.S. Pat. No. 6,465,66 to Essenpreis et al., U.S. Pat. No. 6,214,185 to Offenbacher et al., U.S. Pat. No. 5,310,469 to Cunningham et al., and U.S. Pat. No. 5,683,562 to Shaffer et al., U.S. Pat. No. 6,579,690 to Bonnecaze et al., U.S. Pat. No. 6,484,46 to Say et al., U.S. Pat. No. 6,512,939 to Colvin et al., U.S. Pat. No. 6,424,847 to Mastrototaro et al., U.S. Pat. No. 6,424,847 to Mastrototaro et al., for example. Each of the above patents are incorporated in their entirety herein by reference and are not inclusive of all applicable analyte sensors; in general, it should be understood that the disclosed examples are applicable to a variety of analyte sensor configurations. in other examples of sensor systems including biointerface/drug release layer(s), the sensor may be a planar or substantially planar sensor.

### Exemplary Multi-Analyte Sensor Membrane Configurations

Continuous multi-analyte sensors with various membrane configurations suitable for facilitating signal transduction corresponding to analyte concentrations, either simultaneously, intermittently, and/or sequentially are provided. In one example, such sensors can be configured using a signal transducer, comprising one or more transducing elements (“TL”). Such continuous multi-analyte sensor can employ various transducing means, for example, amperometry, voltametric, potentiometry, and impedimetric methods, among other techniques.

In one example, the transducing element comprises one or more membranes that can comprise one or more layers and or domains, each of the one or more layers or domains can independently comprise one or more signal transducers, e.g., enzymes, RNA, DNA, aptamers, binding proteins, etc. As used herein, transducing elements includes enzymes, ionophores, RNA, DNA, aptamers, binding proteins and are used interchangeably.

In one example, the transducing element is present in one or more membranes, layers, or domains formed over a sensing region. In one example, such sensors can be configured using one or more enzyme domains, e.g., membrane domains including enzyme domains, also referred to as EZ layers (“EZLs”), each enzyme domain may comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

In one example, the continuous multi-analyte sensor uses one or more of the following analyte-substrate/enzyme pairs: for example, sarcosine oxidase in combination with creatinine amidohydrolase, creatine amidohydrolase being employed for the sensing of creatinine. Other examples of analytes/oxidase enzyme combinations that can be used in the sensing region include, for example, alcohol/alcohol oxidase, cholesterol/cholesterol oxidase, glactose:galactose/galactose oxidase, choline/choline oxidase, glutamate/glutamate oxidase, glycerol/glycerol-3phosphate oxidase (or glycerol oxidase), bilirubin/bilirubin oxidase, ascorbic/ascorbic acid oxidase, uric acid/uric acid oxidase, pyruvate/pyruvate oxidase, hypoxanthine:xanthine/xanthine oxidase, glucose/glucose oxidase, lactate/lactate oxidase, L-amino acid oxidase, and glycine/sarcosine oxidase. Other analyte-substrate/enzyme pairs can be used, including such analyte-substrate/enzyme pairs that comprise genetically altered enzymes, immobilized enzymes, mediator-wired enzymes, dimerized and/or fusion enzymes.

### NAD Based Multi-Analyte Sensor Platform

Nicotinamide adenine dinucleotide (NAD(P)⁺/NAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD(P)⁺) and reduced form (NAD(P)H) (H=hydrogen). The reaction of NAD+ and NADH is reversible, thus, the coenzyme can continuously cycle between the NAD(P)⁺/and NAD(P)H forms essentially without being consumed.

In one example, one or more enzyme domains of the sensing region of the presently disclosed continuous multi-analyte sensor device comprise an amount of NAD+ or NADH for providing transduction of a detectable signal corresponding to the presence or concentration of one or more analytes. In one example, one or more enzyme domains of the sensing region of the presently disclosed continuous multi-analyte sensor device comprise an excess amount of NAD+ or NADH for providing extended transduction of a detectable signal corresponding to the presence or concentration of one or more analytes.

In one example, NAD, NADH, NAD⁺, NAD(P)⁺, ATP, flavin adenine dinucleotide (FAD), magnesium (Mg++), pyrroloquinoline quinone (PQQ), and functionalized derivatives thereof can be used in combination with one or more enzymes in the continuous multi-analyte sensor device. In one example, NAD, NADH, NAD+, NAD(P)⁺, ATP, flavin adenine dinucleotide (FAD), magnesium (Mg⁺⁺), pyrroloquinoline quinone (PQQ), and functionalized derivatives are incorporated in the sensing region. In one example, NAD, NADH, NAD⁺, NAD(P)⁺, ATP, flavin adenine dinucleotide (FAD), magnesium (Mg⁺⁺), pyrroloquinoline quinone (PQQ), and functionalized derivatives are dispersed or distributed in one or more membranes or domains of the sensing region.

In one aspect of the present disclosure, continuous sensing of one or more or two or more analytes using NAD+ dependent enzymes is provided in one or more membranes or domains of the sensing region. In one example, the membrane or domain provides retention and stable recycling of NAD+ as well as mechanisms for transducing NADH oxidation or NAD+ reduction into measurable current with amperometry. In one example, described below, continuous, sensing of multi-analytes, either reversibly bound or at least one of which are oxidized or reduced by NAD+ dependent enzymes, for example, ketones (beta-hydroxybutyrate dehydrogenase), glycerol (glycerol dehydrogenase), cortisol (11β-hydroxysteroid dehydrogenase), glucose (glucose dehydrogenase), alcohol (alcohol dehydrogenase), aldehydes (aldehyde dehydrogenase), and lactate (lactate dehydrogenase) is provided. In other examples, described below, membranes are provided that enable the continuous, on-body sensing of multiple analytes which utilize FAD-dependent dehydrogenases, such as fatty acids (Acyl-CoA dehydrogenase).

Exemplary configurations of one or more membranes or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 7A. With reference to FIG. 7B, one or more optional layers may be positioned between the electrode surface and the one or more enzyme domains. For example, one or more interference domains (also referred to as “interferent blocking layer”) can be used to reduce or eliminate signal contribution from undesirable species present, or one or more electrodes (not shown) can used to assist with wetting, system equilibrium, and/or start up. As shown in FIGS. 6A, 6B, one or more of the membranes provides a NAD+ reservoir domain providing a reservoir for NAD+. In one example, one or more interferent blocking membranes is used, and a potentiostat is utilized to measure H₂O₂ production or O2 consumption of an enzyme such as or similar to NADH oxidase, the NAD+ reservoir and enzyme domain positions can be switched, to facilitate better consumption and slower unnecessary outward diffusion of excess NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein; and U.S. Provisional Patent Application No. 63/291,726, “MEDIATOR-TETHERED NAD(H) FOR KETONE SENSING,” filed Dec. 20, 2021, and incorporated by reference in its entirety herein.

In one example, one or more mediators that are optimal for NADH oxidation are incorporated in the one or more electrode domains or enzyme domains. In one example, organic mediators, such as phenanthroline dione, or nitrosoanilines are used. In another example, metallo-organic mediators, such as ruthenium-phenanthroline-dione or osmium(bpy)2Cl, polymers containing covalently coupled organic mediators or organometallic coordinated mediators polymers for example polyvinylimidizole-Os(bpy)2Cl, or poly vinylpyridine-organometallic coordinated mediators (including ruthenium-phenanthroline dione) are used. Other mediators can be used as discussed further below.

In humans, serum levels of beta-hydroxybutyrate (BHB) are usually in the low micromolar range but can rise up to about 6-8 mM. Serum levels of BHB can reach 1-2 mM after intense exercise or consistent levels above 2 mM are reached with a ketogenic diet that is almost devoid of carbohydrates. Other ketones are present in serum, such as acetoacetate and acetone, however, most of the dynamic range in ketone levels is in the form of BHB. Thus, monitoring of BHB, e.g., continuous monitoring is useful for providing health information to a user or health care provider.

Thus, an exemplary continuous ketone analyte detection employing electrode-associated mediator/NAD+/dehydrogenase, for example, beta-hydroxybutyrate dehydrogenase (HBDH) for continuous monitoring of BHB is provided. In one example, a continuous ketone sensor configuration, capable of monitoring BHB, may include a mediator/NAD+/dehydrogenase are present adjacent to the electrode surface. Alternatively, for example, multiple enzyme domains can be used in an enzyme layer, with the mediator/NAD+ comprising layer being more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or reduce migration from the sensing region, for example the NAD+ is dimerized using its C6 terminal amine with any amine-reactive crosslinker, or NAD+ is immobilized to a polymer from its C6 terminal amine. In one example, mediator polymer containing organic mediators, or organometallic coordinated mediator polymers are covalently or otherwise operably coupled to the electrode are used. In other examples, NAD+ may be electrografted to an electroactive surface (e.g., a working electrode). In one example, the electrografted NAD+ is enzyme-active. In another example, the electrografted NAD+ is not enzyme-active.

In some examples, the flux of reactant/co-reactant, such as oxygen through the sensing region has little if any effect on the transduced signal. In the configuration above, there is no consumption of oxygen or production of hydrogen peroxide, rather, direct transfer of electrons from the enzymes to the electrode surface for signal transduction. Thus, notwithstanding endogenous electroactive species such as ascorbate and urate, the need to preferentially attenuate flux of analyte relative to such other reactants such as oxygen and peroxide is reduced or eliminated. For example, homogeneous polymer which have controlled mesh size can be used. In other examples, the sensing region comprises one or more enzyme that is oxygen dependent, and oxygen flux is maximized, for example, including silicone, polysiloxane or copolymers. Other membranes can be used, e.g., positioned in-between or above the aforementioned EZL's or NAD+ reservoirs, for example, drug releasing and/or biointerface layers.

In one example, the diaphorase is electrically coupled to the electrode with organometallic coordinated mediator polymer. In another example, the diaphorase is covalently coupled to the electrode with an organometallic coordinated mediator polymer.

Alternatively, multiple enzyme domains can be used in an enzyme layer, for example, separating the electrode-associated diaphorase (closest to the electrode surface) from the more distal adjacent NAD+ or the dehydrogenase enzyme, to essentially decouple NADH oxidation from analyte (ketone) oxidation. Alternatively, NAD+ can be more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, NAD+ may be covalently coupled to an aspect of the enzyme domain having a higher molecular weight than the NAD+ which may improve a stability profile of the NAD+, improving the ability to retain and/or immobilize the NAD+ in the enzyme domain. For example, dextran-NAD.

In yet another example, transduced signal from a transducing element for a continuous ketone (and one or more other analytes) sensor configuration can be provided using the oxidation of NADH oxidase enzyme for the formation of hydrogen peroxide at electrode surface as the signal transducing species. In this configuration, electrode surface, membranes, layers, or domains that selectively reduces flux of analyte and NAD+, while allowing high flux of oxygen into the sensing region can be used. Thus, one or more interference domains are used, for example, NAFION™ or alternating layers of polyallylamine and polyacrylate acid, etc. In one example, the HBDH and one or more other analyte-specific oxidase enzymes are present in the same or different enzyme domain, and either can be immobilized.

In one example, the NAD+ may be coupled or non-coupled to a polymer or physically entrapped therein, and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, superoxide dismutase (SOD) can be included in the configuration, for example, in the same enzyme domain as NADH for scavenging free radicals generated by NADH oxidase and thus improving signal stability and sensor performance. In the above configuration, the transduced signal is oxygen dependent, and oxygen flux is maximized, for example, using homogeneous polymer membranes which have controlled mesh size, and/or including silicone, polysiloxane or copolymers in the one or more enzyme domains. In one example, where the signal is non-oxygen dependent, a resistance domain is employed to attenuate the flux of analyte(s) into the EZL, so that the sensor response remains linear throughout the physiological range of the target analyte(s). A “target” analyte as discussed herein is an analyte intended to be detected by the sensor systems discussed herein. One or more target analytes may be detected and analyzed using the sensor systems discussed herein.

In one example, the sensing region comprises one or more NADH:acceptor oxidoreductases and one or more NAD-dependent dehydrogenases. In one example, sensing region comprises one or more NADH:acceptor oxidoreductases and one or more NAD(P)-dependent dehydrogenases with NAD(P)+ or NAD(P)H as cofactors present in sensing region. In one example, the sensing region comprises an amount of diaphorase.

In one example, a ketone sensing configuration suitable for combination with another analyte sensing configuration is provided. Thus, an EZL layer of about 1-20 um thick is prepared by presenting a EZL solution composition in 10 mM HEPES in water having about 20 uL 500 mg/mL HBDH, about 20 uL [500 mg/mL NAD(P)H, 200 mg/mL polyethylene glycol-diglycol ether (PEG-DGE) of about 400 MW], about 20 uL 500 mg/mL diaphorase, about 40 uL 250 mg/mL poly vinyl imidazole-osmium bis(2,2′-bipyridine)chloride (PVI-Os(bpy)2Cl) to a substrate such as a working electrode, so as to provide, after drying, about 15-40% by weight HBDH, about 5-30% diaphorase about 5-30% NAD(P)H, about 10-50% PVI-Os(bpy)2Cl and about 1-12% PEG-DGE(400 MW). The substrates discussed herein that may include working electrodes may be formed from gold, platinum, palladium, rhodium, iridium, titanium, tantalum, chromium, and/or alloys or combinations thereof, or carbon (e.g., graphite, glassy carbon, carbon nanotubes, graphene, or doped diamond, as well as combinations thereof.

To the above enzyme domain was contacted a resistance domain, also referred to as a resistance layer (“RL”). In one example, the RL comprises about 55-100% polyvinyl pyrrolidone (PVP), and about 0.1-45% PEG-DGE. In another example, the RL comprises about 75-100% PVP, and about 0.3-25% PEG-DGE. In yet another example, the RL comprises about 85-100% PVP, and about 0.5-15% PEG-DGE. In yet another example, the RL comprises essentially 100% PVP.

The exemplary continuous ketone sensor as depicted in FIGS. 7A, 7B comprising NAD(P)H reservoir domain is configured so that NAD(P)H is not rate-limiting in any of the enzyme domains of the sensing region. In one example, the loading of NAD(P)H in the NAD(P)H reservoir domain is greater than about 20%, 30%, 40% or 50% w/w. The one or more of the membranes or portions of one or more membrane domains (hereinafter also referred to as “membranes”) may also contain a polymer or protein binder, such as zwitterionic polyurethane, and/or albumin. Alternatively, in addition to NAD(P)H, the membrane may contain one or more analyte specific enzymes (e.g. HBDH, glycerol dehydrogenase, etc.), so that optionally, the NAD(P)H reservoir membrane also provides a catalytic function. In one example, the NAD(P)H is dispersed or distributed in or with a polymer (or protein), and may be crosslinked to an extent that still allows adequate enzyme/cofactor functionality and/or reduced NAD(P)H flux within the domain.

In one example, NADH oxidase enzyme alone or in combination with superoxide dismutase (SOD) is used in the one or more membranes of the sensing region. In one example, an amount of superoxide dismutase (SOD) is used that is capable of scavenging some or most of one or more free radicals generated by NADH oxidase. In one example, NADH oxidase enzyme alone or in combination with superoxide dismutase (SOD) is used in combination with NAD(P)H and/or a functionalized polymer with NAD(P)H immobilized onto the polymer from a C6 terminal amine in the one or more membranes of the sensing region.

In one example, the NAD(P)H is immobilized to an extent that maintains NAD(P)H catalytic functionality. In one example, dimerized NAD(P)H is used to entrap NAD(P)H within one or more membranes by crosslinking their respective C6 terminal amine together with appropriate amine-reactive crosslinker such as glutaraldehyde or PEG-DGE or polycarbodiimide crosslinker.

The aforementioned continuous ketone sensor configurations can be adapted to other analytes or used in combination with other sensor configurations. For example, analyte(s)-dehydrogenase enzyme combinations can be used in any of the membranes of the sensing region include; glycerol (glycerol dehydrogenase); cortisol (11β-hydroxysteroid dehydrogenase); glucose (glucose dehydrogenase); alcohol (alcohol dehydrogenase); aldehydes (aldehyde dehydrogenase); and lactate (lactate dehydrogenase).

In one example, a semipermeable membrane is used in the sensing region or adjacent thereto or adjacent to one or more membranes of the sensing region so as to attenuate the flux of at least one analyte or chemical species. In one example, the semipermeable membrane attenuates the flux of at least one analyte or chemical species so as to provide a linear response from a transduced signal. In another example, the semipermeable membrane prevents or eliminates the flux of NAD(P)H out of the sensing region or any membrane or domain. In one example, the semipermeable membrane can be an ion selective membrane selective for an ion analyte of interest, such as ammonium ion.

In another example, a continuous multi-analyte sensor configuration comprising one or more enzymes and/or at least one cofactor was prepared. FIG. 7C depicts this exemplary configuration, of an enzyme domain 750 comprising an enzyme (Enzyme) with an amount of cofactor (Cofactor) that is positioned proximal to at least a portion of a working electrode (“WE”) surface, where the WE comprises an electrochemically reactive surface. In one example, a second membrane 751 comprising an amount of cofactor is positioned adjacent the first enzyme domain. The amount of cofactor in the second membrane can provide an excess for the enzyme, e.g., to extend sensor life. One or more resistance domains 752 (“RL”) are positioned adjacent the second membrane (or can be between the membranes). The RL can be configured to block diffusion of cofactor from the second membrane. Electron transfer from the cofactor to the WE transduces a signal that corresponds directly or indirectly to an analyte concentration.

FIG. 7D depicts an alternative enzyme domain configuration comprising a first membrane 751 with an amount of cofactor that is positioned more proximal to at least a portion of a WE surface. Enzyme domain 750 comprising an amount of enzyme is positioned adjacent the first membrane.

In the membrane configurations depicted in FIGS. 7C, 7D, production of an electrochemically active species in the enzyme domain diffuses to the WE surface and transduces a signal that corresponds directly or indirectly to an analyte concentration. In some examples, the electrochemically active species comprises hydrogen peroxide. For sensor configurations that include a cofactor, the cofactor from the first layer can diffuse to the enzyme domain to extend sensor life, for example, by regenerating the cofactor. For other sensor configurations, the cofactor can be optionally included to improve performance attributes, such as stability. For example, a continuous ketone sensor can comprise NAD(P)H and a divalent metal cation, such as Mg⁺². One or more resistance domains RL can be positioned adjacent the second membrane (or can be between the layers). The RL can be configured to block diffusion of cofactor from the second membrane and/or interferents from reaching the WE surface. Other configurations can be used in the aforementioned configuration, such as electrode, resistance, bio-interfacing, and drug releasing membranes, layers or domains. In other examples, continuous analyte sensors including one or more cofactors that contribute to sensor performance.

FIG. 7E depicts another continuous multi-analyte membrane configuration, where {beta}-hydroxybutyrate dehydrogenase BHBDH in a first enzyme domain 753 is positioned proximate to a working electrode WE and second enzyme domain 754, for example, comprising alcohol dehydrogenase (ADH) and NADH is positioned adjacent the first enzyme domain. One or more resistance domains RL 752 may be deployed adjacent to the second enzyme domain 754. In this configuration, the presence of the combination of alcohol and ketone in serum works collectively to provide a transduced signal corresponding to at least one of the analyte concentrations, for example, ketone. Thus, as the NADH present in the more distal second enzyme domain consumes alcohol present in the serum environment, NADH is oxidized to NAD(P)H that diffuses into the first membrane layer to provide electron transfer of the BHBDH catalysis of acetoacetate ketone and transduction of a detectable signal corresponding to the concentration of the ketone. In one example, an enzyme can be configured for reverse catalysis and can create a substrate used for catalysis of another enzyme present, either in the same or different layer or domain. Other configurations can be used in the aforementioned configuration, such as electrode, resistance, bio-interfacing, and drug releasing membranes, layers, or domains. Thus, a first enzyme domain that is more distal from the WE than a second enzyme domain may be configured to generate a cofactor or other element to act as a reactant (and/or a reactant substrate) for the second enzyme domain to detect the one or more target analytes.

### One-Working-Electrode Configurations for Dual Analyte Detection

In one example, at least a dual enzyme domain configuration in which each layer contains one or more specific enzymes and optionally one or more cofactors is provided. In a broad sense, one example of a continuous multi-analyte sensor configuration is depicted in FIG. 8A where a first membrane 855 (EZL1) comprising at least one enzyme (Enzyme 1) of the at least two enzyme domain configuration is proximal to at least one surface of a WE. One or more analyte-substrate enzyme pairs with Enzyme 1 transduces at least one detectable signal to the WE surface by direct electron transfer or by mediated electron transfer that corresponds directly or indirectly to an analyte concentration. Second membrane 856 (EZL2) with at least one second enzyme (Enzyme 2) is positioned adjacent 855 ELZ1, and is generally more distal from WE than EZL1. One or more resistance domains (RL) 852 can be provided adjacent EZL2 856, and/or between EZL1 855 and EZL2 856. The different enzymes catalyze the transformation of the same analyte, but at least one enzyme in EZL2 856 provides hydrogen peroxide and the other at least one enzyme in EZL1 855 does not provide hydrogen peroxide. Accordingly, each measurable species (e.g., hydrogen peroxide and the other measurable species that is not hydrogen peroxide) generates a signal associated with its concentration.

For example, in the configuration shown in FIG. 8A, a first analyte diffuses through RL 852 and into EZL2 856 resulting in peroxide via interaction with Enzyme 2. Peroxide diffuses at least through EZL1 855 to WE and transduces a signal that corresponds directly or indirectly to the first analyte concentration. A second analyte, which is different from the first analyte, diffuses through RL 852 and EZL2 856 and interacts with Enzyme 1, which results in electron transfer to WE and transduces a signal that corresponds directly or indirectly to the second analyte concentration.

As shown in FIG. 8B, the above configuration is adapted to a conductive wire electrode construct, where at least two different enzyme-containing layers are constructed on the same WE with a single active surface. In one example, the single WE is a wire, with the active surface positioned about the longitudinal axis of the wire. In another example, the single WE is a conductive trace on a substrate, with the active surface positioned about the longitudinal axis of the trace. In one example, the active surface is substantially continuous about a longitudinal axis or a radius.

In the configuration described above, at least two different enzymes can be used and catalyze the transformation of different analytes, with at least one enzyme in EZL2 856 providing hydrogen peroxide and the at least other enzyme in EZL1 855 not providing hydrogen peroxide, e.g., providing electron transfer to the WE surface corresponding directly or indirectly to a concentration of the analyte.

In one example, an inner layer of the at least two enzyme domains EZL1, EZL2 855, 856 comprises at least one immobilized enzyme in combination with at least one mediator that can facilitate lower bias voltage operation of the WE than without the mediator. In one example, for such direct electron transductions, a potential P1 is used. In one example, at least a portion of the inner layer EZL1 855 is more proximal to the WE surface and may have one or more intervening electrode domains and/or overlaying interference and/or bio-interfacing and/or drug releasing membranes, provided that the at least one mediator can facilitate low bias voltage operation with the WE surface. In another example, at least a portion of the inner layer EZL1 855 is directly adjacent the WE.

The second layer of at least dual enzyme domain (the outer layer EZL2 856) of FIG. 8B contains at least one enzyme that result in one or more catalysis reactions that eventually generate an amount of hydrogen peroxide that can electrochemically transduce a signal corresponding to the concentration of the analyte(s). In one example, the generated hydrogen peroxide diffuses through layer EZL2 856 and through the inner layer EZL1 855 to reach the WE surface and undergoes redox at a potential of P2, where P2≠P1. In this way electron transfer and electrolysis (redox) can be selectively controlled by controlling the potentials P1, P2 applied at the same WE surface. Any applied potential durations can be used for P1, P2, for example, equal/periodic durations, staggered durations, random durations, as well as various potentiometric sequences, cyclic voltammetry etc. In some examples, impedimetric sensing may be used. In one example, a phase shift (e.g., a time lag) may result from detecting two signals from two different working electrodes, each signal being generated by a different EZL (EZL1, EZL2, 855, 856) associated with each electrode. The two (or more) signals can be broken down into components to detect the individual signal and signal artifacts generated by each of EZL1 855 and EZL2 856 in response to the detection of two analytes. In some examples, each EZL detects a different analyte. In other examples, both EZLs detect the same analyte.

In another alternative exemplary configuration, as shown in FIGS. 8C, 8D a multienzyme domain configuration as described above is provided for a continuous multi-analyte sensor device using a single WE with two or more active surfaces is provided. In one example, the multienzyme domain configurations discussed herein are formed on a planar substrate. In another example, the single WE is coaxial, e.g., configured as a wire, having two or more active surfaces positioned about the longitudinal axis of the wire. Additional wires can be used, for example, as a reference and/or counter electrode. In another example, the single WE is a conductive trace on a substrate, with two or more active surfaces positioned about the longitudinal axis of the trace. At least a portion of the two or more active surfaces are discontinuous, providing for at least two physically separated WE surfaces on the same WE wire or trace. (e.g., WE1, WE2). In one example, the first analyte detected by WE1 is glucose, and the second analyte detected by WE2 is lactate. In another example, the first analyte detected by WE1 is glucose, and the second analyte detected by WE2 is ketones.

Thus, FIGS. 8C, 8D depict exemplary configurations of a continuous multi-analyte sensor construct in which EZL1 855, EZL2 856 and RL 852 (resistance domain) as described above, arranged, for example, by sequential dip-coating techniques, over a single coaxial wire comprising spatially separated electrode surfaces WE1, WE2. One or more parameters, independently, of the enzyme domains, resistance domains, etc., can be controlled along the longitudinal axis of the WE, for example, thickness, length along the axis from the distal end of the wire, etc. In one example, at least a portion of the spatially separated electrode surfaces are of the same composition. In another example, at least a portion of the spatially separated electrode surfaces are of different composition. In FIGS. 8C, 8D, WE1 represents a first working electrode surface configured to operate at P1, for example, and is electrically insulated from second working electrode surface WE2 that is configured to operate at P2, and RE represents a reference electrode RE electrically isolated from both WE1, WE2. One resistance domain is provided in the configuration of FIG. 8C that covers the reference electrode and WE1, WE2. An additional resistance domain is provided in the configuration of FIG. 8D that covers extends over essentially WE2 only. Additional electrodes, such as a counter electrode can be used. Such configurations (whether single wire or dual wire configurations) can also be used to measure the same analyte using two different techniques. Using different signal generating sequences as well as different RLs, the data collected from two different modes of measurements provides increased fidelity, improved performance and device longevity. A non-limiting example is a glucose oxidase (H₂O₂ producing) and glucose dehydrogenase (electrically coupled) configuration. Measurement of Glucose at two potentials and from two different electrodes provides more data points and accuracy. Such approaches may not be needed for glucose sensing, but can be applied across the biomarker sensing spectrum of other analytes, alone or in combination with glucose sensing, such as ketone sensing, ketone/lactate sensing, and ketone/glucose sensing.

In an alternative configuration of that depicted in FIGS. 8C, 8D, two or more wire electrodes, which can be colinear, wrapped, or otherwise juxtaposed, are presented, where WE1 is separated from WE2, for example, with another elongated shaped electrode. An insulating layer electrically isolates WE1 from WE2. In this configuration, independent electrode potentials can be applied to the corresponding electrode surfaces, where the independent electrode potentials can be provided simultaneously, sequentially, or randomly to WE1, WE2. In one example, electrode potentials presented to the corresponding electrode surfaces WES1, WES2, are different. One or more additional electrodes can be present such as a reference electrode and/or a counter electrode. In one example, WES2 is positioned longitudinally distal from WES1 in an elongated arrangement. Using, for example, dip-coating methods, WES1 and WES2 are coated with enzyme domain EZL1, while WES2 is coated with different enzyme domain EZL2. Based on the dipping parameters, or different thickness of enzyme domains, multi-layered enzyme domains, each layer independently comprising different loads and/or compositions of enzyme and/or cofactors, mediators can be employed. Likewise, one or more resistance domains (RL) can be applied, each can be of a different thickness along the longitudinal axis of the electrode, and over different electrodes and enzyme domains by controlling dip length and other parameters, for example. With reference to FIG. 8D, such an arrangement of RL's is depicted, where an additional RL 852′ is adjacent WES2 but substantially absent from WES1.

In one example of measuring two different analytes, the above configuration comprising enzyme domain EZL1 855 comprising one or more enzyme(s) and one or more mediators for at least one enzyme of EZL1 to provide for direct electron transfer to the WES1 and determine a concentration of at least a first analyte. In addition, enzyme domain EZL2 856 can comprise at least one enzyme that provides peroxide (e.g., hydrogen peroxide) or consumes oxygen during catalysis with its substrate. The peroxide or the oxygen produced in EZL2 856 migrates to WES2 and provides a detectable signal that corresponds directly or indirectly to a second analyte. For example, WES2 can be carbon, wired to glucose dehydrogenase to measure glucose, while WES1 can be platinum, that measures peroxide produced from lactate oxidase/lactate in EZL2 856. The combinations of electrode material and enzyme(s) as disclosed herein are examples and non-limiting.

In one example, the potentials of P1 and P2 can be separated by an amount of potential so that both signals (from direct electron transfer from EZL1 855 and from hydrogen peroxide redox at WE) can be separately activated and measured. In one example, the electronic module of the sensor can switch between two sensing potentials continuously in a continuous or semi-continuous periodic manner, for example a period (t1) at potential P1, and period (t2) at potential P2 with optionally a rest time with no applied potential. Signal extracted can then be analyzed to measure the concentration of the two different analytes. In another example, the electronic module of the sensor can undergo cyclic voltammetry, providing changes in current when swiping over potentials of P1 and P2, which can be correlated to transduced signals coming from either direct electron transfer or electrolysis of hydrogen peroxide, respectively. In one example, the modality of sensing is non limiting and can include different amperometry techniques, e.g., cyclic voltammetry. In one example, an alternative configuration is provided but hydrogen peroxide production in EZL2 is replaced by another suitable electrolysis compound that maintains the P2≠P1 relationship, such as oxygen, and at least one enzyme-substrate combination that provide the other electrolysis compound.

For example, a continuous multi-analyte sensor configuration, for choline and glucose, in which enzyme domains EZ1 855, EZ2 856 were associated with different WEs, e.g., platinum WE2, and gold WE1 was prepared. In this exemplary case, EZL1 855 contained glucose oxidase and a mediator coupled to WE1 to facilitate electron direct transfer upon catalysis of glucose, and EZL2 856 contained choline oxidase that will catalyze choline and generate hydrogen peroxide for electrolysis at WE2. The EZL's were coated with resistance domains; upon cure and readiness they underwent cyclic voltammetry in the presence of glucose and choline. A wired glucose oxidase enzyme to a gold electrode is capable of transducing signal at 0.2 volts, therefore, by analyzing the current changes at 0.2 volts, the concentration of glucose can be determined. The data also demonstrates that choline concentration is also inferentially detectable at the WE2 platinum electrode if the CV trace is analyzed at the voltage P2.

In one example, either electrode WE1 or WE2 can be, for example, a composite material, for example a gold electrode with platinum ink deposited on top, or other coatings may be employed such including a carbon/platinum mix, and or traces of carbon on top of platinum, or a porous carbon coating on a platinum surface. In one example, with the electrode surfaces containing two distinct materials, for example, carbon used for the wired enzyme and electron transfer, while platinum can be used for hydrogen peroxide redox and detection. As shown in FIG. 8E, an example of such composite electrode surfaces is shown, in which an extended platinum covered wire 857 is half coated with carbon 858, to facilitate multi sensing on two different surfaces of the same electrode. In one example WE2 can be grown on or extend from a portion of the surface or distal end of WE1, for example, by vapor deposition, sputtering, or electrolytic deposition and the like.

Additional examples include a composite electrode material that may be used to form one or both of WE1 and WE2. In one example, a platinum-carbon electrode WE1, comprising EZL1 with glucose dehydrogenase is wired to the carbon surface, and outer EZL2 comprising lactate oxidase generating hydrogen peroxide that is detectable by the platinum surface of the same WE1 electrode. Other examples of this configuration can include ketone sensing (beta-hydroxybutyrate dehydrogenase electrically coupled enzyme in EZL1 855) and glucose sensing (glucose oxidase in EZL2 856). Other membranes can be used in the aforementioned configuration, such as electrode, resistance, bio-interfacing, and drug releasing membranes. In other examples, one or both of the working electrodes (WE1, WE2) may be gold-carbon (Au—C), palladium-carbon (Pd—C), iridium-carbon (Ir—C), rhodium-carbon (Rh—C), or ruthenium-carbon (Ru—C). In some examples, the carbon in the working electrodes discussed herein may instead or additionally include graphene, graphene oxide, or other materials suitable for forming the working electrodes, such as commercially available carbon ink.

In one example, the first layer EZL1 855 comprises oxidase enzymes that do not produce hydrogen peroxide. Such enzymes include, but are not limited to lactate dehydrogenase, glucose dehydrogenase, beta-hydroxybutyrate dehydrogenase, diaphorase, and the like. In one example, these dehydrogenase enzymes are wired to at least a portion of the WE1 electrode so as to at reduce or eliminate cross talk, reduce potential, and minimize or eliminate interfering signals. In one example the EZL1 855 can comprise any enzyme that can provide electron transfer while wired or covalently coupled to the electrode surface or in the presence of any type of redox mediator, and the EZL2 856 can comprise any oxidoreductase enzymes that produces hydrogen peroxide or other suitable compound that will under redox or electrolysis at the electrode surface at the applied potential.

In one example, the aforementioned continuous choline sensor configurations can be combined with any one of the aforementioned continuous alcohol sensor configurations, continuous uric acid sensor configurations, continuous cholesterol sensor configurations, continuous bilirubin/ascorbic acid sensor configurations, continuous ketone, ketone and glucose, or ketone and lactate, as well as other sensor configurations to provide a continuous multi-analyte sensor device as further described below. This continuous multi-analyte sensor device can further include continuous glucose monitoring capability.

### Example Embodiments

Embodiment 1: Embodiments of the present disclosure provide a method for providing euglycemic diabetic ketoacidosis decision support to a patient, the method including monitoring two or more more analytes of the patient during one or more time periods to obtain analyte data, the two or more analytes including at least glucose and ketone and the analyte data comprising glucose data and ketone data. The method further includes processing the analyte data to determine analyte metrics, including at least glucose metrics and ketone metrics and generating a treatment recommendation based, at least in part, on the analyte metrics.

Embodiment 2: The method of Embodiment 1 further including receiving patient treatment data corresponding to the patient.

Embodiment 3: The method of Embodiment 2, wherein the patient treatment data comprises medication information corresponding to a pharmacologic agent and the treatment recommendation comprises at least one of: a change in dosage of the pharmacologic agent or a change in frequency of consumption of the pharmacologic agent.

Embodiment 4: The method of Embodiment 2, further comprising generating a euglycemic diabetic ketoacidosis prediction using the analyte metrics and the patient treatment data.

Embodiment 5: The method of Embodiment 4, further comprising generating an alert, alarm, or notification based on at least one of the euglycemic diabetic ketoacidosis prediction or the treatment recommendation.

Embodiment 6: The method of Embodiment 1, wherein the monitoring of the two or more analytes of the patient comprises monitoring of sensor data generated by one or more analyte sensors worn by the patient.

Embodiment 7: Embodiments of the present disclosure provide a non-transitory computer readable medium having instructions stored thereon that, when executed by a processor, cause the processor to perform a method for providing euglycemic diabetic ketoacidosis decision support to a patient, the method comprising monitoring two or more analytes of the patient during one or more time periods to obtain analyte data, the two or more analytes including at least glucose and ketone and the analyte data comprising glucose data and ketone data. The method further includes processing the analyte data to determine analyte metrics, including at least glucose metrics and ketone metrics and generating a treatment recommendation based, at least in part, on the analyte metrics.

Embodiment 8: The non-transitory computer readable medium of Embodiment 7, wherein the method further comprises receiving patient treatment data corresponding to the patient.

Embodiment 9: The non-transitory computer readable medium of Embodiment 8, wherein the patient treatment data comprises medication information corresponding to a pharmacologic agent and the treatment recommendation comprises at least one of: a change in dosage of the pharmacologic agent or a change in frequency of consumption of the pharmacologic agent.

Embodiment 10: The non-transitory computer readable medium of Embodiment 8, wherein the method further comprises generating a euglycemic diabetic ketoacidosis prediction using the analyte metrics and the patient treatment data.

Embodiment 11: The non-transitory computer readable medium of Embodiment 10, wherein the method further comprises generating an alert, alarm, or notification based on at least one of the euglycemic diabetic ketoacidosis prediction or the treatment recommendation.

Embodiment 12: The non-transitory computer readable medium of Embodiment 7, wherein the monitoring of the two or more analytes of the patient comprises monitoring of sensor data generated by one or more analyte sensors worn by the patient.

Embodiment 13: Embodiments of the present disclosure provide a monitoring system comprising a continuous analyte sensor configured to generate analyte measurements associated with analyte levels of a patient and a sensor electronics module coupled to the continuous analyte sensor and configured to receive and process the analyte measurements.

Embodiment 14: The monitoring system of Embodiment 13, wherein the continuous analyte sensor comprises a substrate, a working electrode disposed on the substrate, and a reference electrode disposed on the substrate, wherein the analyte measurements generated by the continuous analyte sensor are determined using electrochemical methods, at least in part based on a difference in current produced between the working electrode and the reference electrode.

Embodiment 15: The monitoring system of any of Embodiments 13 or 14, wherein the continuous analyte sensor is a multi-analyte sensor comprising a continuous glucose sensor and a continuous ketone sensor, and the analyte measurements include glucose measurements and ketone measurements.

Embodiment 16: The monitoring system of any of Embodiments 13-15, further comprising a memory comprising executable instructions and one or more processors in data communication with the memory. The one or more processors are configured to execute the executable instructions to receive, from the sensor electronics module, the analyte measurements comprising the glucose measurements and the ketone measurements, process the analyte measurements to determine analyte metrics, including at least glucose metrics and ketone metrics, and generate a treatment recommendation based, at least in part, on the analyte metrics.

Embodiment 17: The monitoring system of any of Embodiments 13-16, wherein the processor is further configured to receive patient treatment data corresponding to the patient.

Embodiment 18: The monitoring system of any of Embodiments 13-17, wherein the patient treatment data comprises medication information corresponding to a pharmacologic agent configured to counteract euglycemic diabetic ketoacidosis and the treatment recommendation comprises at least one of: a change in dosage of the pharmacologic agent or a change in frequency of consumption of the pharmacologic agent.

Embodiment 19: The monitoring system of any of Embodiments 13-18, wherein the pharmacologic agent is an SGLT-2 inhibitor and the treatment recommendation comprises a change in at least the frequency or dosage of the SGLT-2 inhibitor.

Embodiment 20: The monitoring system of any of Embodiments 13-19, wherein the processor is further configured to generate a euglycemic diabetic ketoacidosis prediction using the analyte metrics and the patient treatment data.

Embodiment 21: The monitoring system of any of Embodiments 13-20, wherein the processor is further configured to generate an alert or alarm based on at least one of the euglycemic diabetic ketoacidosis prediction or the treatment recommendation.

Embodiment 22: The monitoring system of any of Embodiments 13-21, further comprising one or more non-analyte sensors, wherein the processor is further configured to receive non-analyte sensor data generated for the patient using one or more non-analyte sensors, wherein the treatment recommendation is further based on non-analyte sensor data.

Embodiment 23: The monitoring system of any of Embodiments 13-22, wherein the one or more non-analyte sensors comprise at least one of an insulin pump, an ECG sensor, a heart rate monitor, a blood pressure sensor, a respiratory sensor, a thermometer, an oxygenated hemoglobin sensor, an activity tracker, a peritoneal dialysis machine, or a hemodialysis machine.

Embodiment 24: The monitoring system of any of Embodiments 13-23, wherein the processor is further configured to generate a euglycemic diabetic ketoacidosis prediction based, at least in part, on the analyte metrics.

Embodiment 25: The monitoring system of any of Embodiments 13-24, wherein the euglycemic diabetic ketoacidosis prediction comprises at least one of a likelihood or a risk that the patient is experiencing euglycemic diabetic ketoacidosis, a likelihood or a risk that the user will experience euglycemic diabetic ketoacidosis, a presence of euglycemic diabetic ketoacidosis experienced by the patient, or a severity of euglycemic diabetic ketoacidosis experienced by the patient.

Embodiment 26: The monitoring system of any of Embodiments 13-25, wherein the euglycemic diabetic ketoacidosis prediction is determined by a rules-based model, a machine learning model, a Kalman filter, a probabalistic model, or a stochastic model.

Embodiment 27: The monitoring system of any of Embodiments 13-26, wherein the analyte metrics comprise at least one of ketone baseline, ketone level maximum, ketone level minimum, ketone level rates of change, ketone clearance rates, ketone trends, ketone time-in-range, glucose level rates of change, glucose trends, glycemic variability, glucose clearance, or glucose time-in-range.

Embodiment 28: Embodiments of the present disclosure provide a method for providing euglycemic diabetic ketoacidosis decision support to a patient, the method including monitoring two or more more analytes of the patient during one or more time periods to obtain analyte data, the two or more analytes including at least glucose and ketone and the analyte data comprising glucose data and ketone data. The method further includes processing the analyte data to determine analyte metrics, including at least glucose metrics and ketone metrics and generating a treatment recommendation based, at least in part, on the analyte metrics.

Embodiment 29: The method of Embodiment 28, further including receiving patient treatment data corresponding to the patient.

Embodiment 30: The method of any of Embodiments 28 or 29, wherein the patient treatment data comprises medication information corresponding to a pharmacologic agent and the treatment recommendation comprises at least one of: a change in dosage of the pharmacologic agent or a change in frequency of consumption of the pharmacologic agent.

Embodiment 31: The method of any of Embodiments 28-30, further comprising generating a euglycemic diabetic ketoacidosis prediction using the analyte metrics and the patient treatment data.

Embodiment 32: The method of any of Embodiments 28-31, further comprising generating an alert, alarm, or notification based on at least one of the euglycemic diabetic ketoacidosis prediction or the treatment recommendation.

Embodiment 33: The method of any of Embodiments 28-32, wherein the monitoring of the two or more analytes of the patient comprises monitoring of sensor data generated by one or more analyte sensors worn by the patient.

Embodiment 34: Embodiments of the present disclosure provide a non-transitory computer readable medium having instructions stored thereon that, when executed by a processor, cause the processor to perform a method for providing euglycemic diabetic ketoacidosis decision support to a patient, the method comprising monitoring two or more analytes of the patient during one or more time periods to obtain analyte data, the two or more analytes including at least glucose and ketone and the analyte data comprising glucose data and ketone data. The method further includes processing the analyte data to determine analyte metrics, including at least glucose metrics and ketone metrics and generating a treatment recommendation based, at least in part, on the analyte metrics.

Embodiment 35: The non-transitory computer readable medium of Embodiment 34, wherein the method further comprises receiving patient treatment data corresponding to the patient.

Embodiment 36: The non-transitory computer readable medium of any of Embodiments 34 or 35, wherein the patient treatment data comprises medication information corresponding to a pharmacologic agent and the treatment recommendation comprises at least one of: a change in dosage of the pharmacologic agent or a change in frequency of consumption of the pharmacologic agent.

Embodiment 37: The non-transitory computer readable medium of any of Embodiments 34-36, wherein the method further comprises generating a euglycemic diabetic ketoacidosis prediction using the analyte metrics and the patient treatment data.

Embodiment 38: The non-transitory computer readable medium of any of Embodiments 34-37, wherein the method further comprises generating an alert, alarm, or notification based on at least one of the euglycemic diabetic ketoacidosis prediction or the treatment recommendation.

Embodiment 39: The non-transitory computer readable medium of any of Embodiments 34-38, wherein the monitoring of the two or more analytes of the patient comprises monitoring of sensor data generated by one or more analyte sensors worn by the patient.

### Additional Considerations

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-b-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

What is claimed is:

1. A monitoring system, comprising:
a continuous analyte sensor configured to generate analyte measurements associated with analyte levels of a patient; and
a sensor electronics module coupled to the continuous analyte sensor and configured to receive and process the analyte measurements.

2. The monitoring system of claim 1, wherein the continuous analyte sensor comprises:
a substrate;
a working electrode disposed on the substrate; and
a reference electrode disposed on the substrate, wherein the analyte measurements generated by the continuous analyte sensor are determined using electrochemical methods, at least in part based on a difference in current produced between the working electrode and the reference electrode.

3. The monitoring system of claim 1, wherein:
the continuous analyte sensor is a multi-analyte sensor comprising a continuous glucose sensor and a continuous ketone sensor; and
the analyte measurements include glucose measurements and ketone measurements.

4. The monitoring system of claim 3, further comprising:
a memory comprising executable instructions; and
one or more processors in data communication with the memory and configured to execute the executable instructions to:
receive, from the sensor electronics module, the analyte measurements comprising the glucose measurements and the ketone measurements;
process the analyte measurements to determine analyte metrics, including at least glucose metrics and ketone metrics; and
generate a treatment recommendation based, at least in part, on the analyte metrics.

5. The monitoring system of claim 4, wherein the processor is further configured to:
receive patient treatment data corresponding to the patient.

6. The monitoring system of claim 5, wherein:
the patient treatment data comprises medication information corresponding to a pharmacologic agent configured to counteract euglycemic diabetic ketoacidosis; and
the treatment recommendation comprises at least one of: a change in dosage of the pharmacologic agent or a change in frequency of consumption of the pharmacologic agent.

7. The monitoring system of claim 6, wherein the pharmacologic agent is an SGLT-2 inhibitor and the treatment recommendation comprises a change in at least the frequency or dosage of the SGLT-2 inhibitor.

8. The monitoring system of claim 5, wherein the processor is further configured to:
generate a euglycemic diabetic ketoacidosis prediction using the analyte metrics and the patient treatment data.

9. The monitoring system of claim 8, wherein the processor is further configured to:
generate an alert or alarm based on at least one of the euglycemic diabetic ketoacidosis prediction or the treatment recommendation.

10. The monitoring system of claim 4, further comprising:
one or more non-analyte sensors, wherein the processor is further configured to:
receive non-analyte sensor data generated for the patient using one or more non-analyte sensors, wherein the treatment recommendation is further based on non-analyte sensor data.

11. The monitoring system of claim 10, wherein the one or more non-analyte sensors comprise at least one of an insulin pump, an ECG sensor, a heart rate monitor, a blood pressure sensor, a respiratory sensor, a thermometer, an oxygenated hemoglobin sensor, an activity tracker, a peritoneal dialysis machine, or a hemodialysis machine.

12. The monitoring system of claim 4, wherein the processor is further configured to generate a euglycemic diabetic ketoacidosis prediction based, at least in part, on the analyte metrics.

13. The monitoring system of claim 12, wherein the euglycemic diabetic ketoacidosis prediction comprises at least one of a likelihood or a risk that the patient is experiencing euglycemic diabetic ketoacidosis, a likelihood or a risk that the user will experience euglycemic diabetic ketoacidosis, a presence of euglycemic diabetic ketoacidosis experienced by the patient, or a severity of euglycemic diabetic ketoacidosis experienced by the patient.

14. The monitoring system of claim 13, wherein the euglycemic diabetic ketoacidosis prediction is determined by a rules-based model, a machine learning model, a Kalman filter, a probabalistic model, or a stochastic model.

15. The monitoring system of claim 4, wherein the analyte metrics comprise at least one of ketone baseline, ketone level maximum, ketone level minimum, ketone level rates of change, ketone clearance rates, ketone trends, ketone time-in-range, glucose level rates of change, glucose trends, glycemic variability, glucose clearance, or glucose time-in-range.
